SEQUENCE LISTING (42 sequences)

SEQ ID NO: 1 - protein, 544 aa, Human adenovirus 3
MRRRAVLGGAVVYPEGPPPSYESVMQQQAAMIQPPLEAPFVPPRYLAPTEGRNSIRYSELSPLYDTTKLYLVDNKSADIASLNYQNDHSNFLTTVVQNNDFTPTEASTQTINFDERSRWGGQLKTIMHTNMPNVNEYMFSNKFKARVMVSRKAPEGVTVNDTYDHKEDILKYEWFEFILPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTLAVAEETSEDDDITRGDTYITEKQKREAAAAEVKKELKIQPLEKDSKSRSYNVLEDKINTAYRSWYLSYNYGNPEKGIRSWTLLTTSDVTCGAEQVYWSLPDMMQDPVTFRSTRQVNNYPVVGAELMPVFSKSFYNEQAVYSQQLRQATSLTHVFNRFPENQILIRPPAPTITTVSENVPALTDHGTLPLRSSIRGVQRVTVTDARRRTCPYVYKALGIVAPRVLSSRTF

SEQ ID NO: 2 - protein, 571 aa, Human adenovirus 2
MQRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRSLTKDKQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGDGAGGGNNSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTSQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 3 - protein, 535 aa, Human adenovirus 4
MMRRAYPEGPPPSYESVMQQAMAAAAAIQPPLEAPYVPPRYLAPTEGRNSIRYSELTPLYDTTRLYLVDNKSADIASLNYQNDHSNFLTTVVQNNDFTPTEASTQTINFDERSRWGGQLKTIMHTNMPNVNQFMYSNKFKARVMVSRKTPNGVTVGDNYDGSQDELKYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLDGGNIPALLDVEAYEKSKEESVAAATTAVATASTEVRDDNFASAAAVAAVKADETKSKIVIQPVEKDSKERSYNVLSDKKNTAYRSWYLAYNYGDRDKGVRSWTLLTTSDVTCGVEQVYWSLPDMMQDPVTFRSTHQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGIVAPRVLSSRTF

SEQ ID NO: 4 - protein, 571 aa, Human adenovirus 5
MRRAAMYEEGPPPSYESVVSAAPVAAALGSPFDAPLDPPFVPPRYLRPTGGRNSIRYSELAPLFDTTRVYLVDNKSTDVASLNYQNDHSNFLTTVIQNNDYSPGEASTQTINLDDRSHWGGDLKTILHTNMPNVNEFMFTNKFKARVMVSRLPTKDNQVELKYEWVEFTLPEGNYSETMTIDLMNNAIVEHYLKVGRQNGVLESDIGVKFDTRNFRLGFDPVTGLVMPGVYTNEAFHPDIILLPGCGVDFTHSRLSNLLGIRKRQPFQEGFRITYDDLEGGNIPALLDVDAYQASLKDDTEQGGGGAGGSNSSGSGAEENSNAAAAAMQPVEDMNDHAIRGDTFATRAEEKRAEAEAAAEAAAPAAQPEVEKPQKKPVIKPLTEDSKKRSYNLISNDSTFTQYRSWYLAYNYGDPQTGIRSWTLLCTPDVTCGSEQVYWSLPDMMQDPVTFRSTRQISNFPVVGAELLPVHSKSFYNDQAVYSQLIRQFTSLTHVFNRFPENQILARPPAPTITTVSENVPALTDHGTLPLRNSIGGVQRVTITDARRRTCPYVYKALGIVSPRVLSSRTF

SEQ ID NO: 5 - protein, 544 aa, Human adenovirus 7
MRRRAVLGGAMVYPEGPPPSYESVMQQQAAMIQPPLEAPFVPPRYLAPTEGRNSIRYSELSPLYDTTKLYLVDNKSADIASLNYQNDHSNFLTTVVQNNDFTPTEASTQTINFDERSRWGGQLKTIMHTNMPNVNEYMFSNKFKARVMVSRKAPEGVIVNDTYDHKEDILKYEWFEFTLPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTLAVAEETSEDDNITRGDTYITEKQKREAAAAEVKKELKIQPLEKDSKSRSYNVLEDKINTAYRSWYLSYNYGNPEKGIRSWTLLTTSDVTCGAEQVYWSLPDMMQDPVTFRSTRQVNNYPVVGAELMPVFSKSFYNEQAVYSQQLRQATSLTHVFNRFPENQILIRPPAPTITTVSENVPALTDHGTLPLRSSIRGVQRVTVTDARRRTCPYVYKALGIVAPRVLSSRTF

SEQ ID NO: 6 - protein, 557 aa, Human adenovirus 11
MRRVVLGGAVVYPEGPPPSYESVMQQQATAVMQSPLEAPFVPPRYLAPTEGRNSIRYSELAPQYDTTRLYLVDNKSADIASLNYQNDHSNFLTTVVQNNDFTPTEASTQTINFDERSRWGGQLKTIMHTNMPNVNEYMFSNNFKARVMVSRKPPEGAAVGDTYDHKQDILEYEWFEFTLPEGNFSVTMTIDLMNNAIIDNYLKVGRQNGVLESDIGVKFDTRNFKLGWDPETKLIMPGVYTYEAFHPDIVLLPGCGVDFTESRLSNLLGIRKKQPFQEGFKILYEDLEGGNIPALLDVDAYENSKKEQKAKIEAAAEAKANIVASDSTRVANAGEVRGDNFAPTPVPTAESLLADVSGGTDVKLTIQPVEKDSKNRSYNVLEDKINTAYRSWYLSYNYGDPEKGVRSWTLLTTSDVTCGAEQVYWSLPDMMQDPVTFRSTRQVSNYPVVGAELMPVFSKSFYNEQAVYSQQLRQSTSLTHVFNRFPENQILIRPPAPTITTVSENVPALTDHGTLPLRSSIRGVQRVTVTDARRRTCPYVYKALGIVAPRVLSSRTF

SEQ ID NO: 7 - protein, 497 aa, Human adenovirus 11
MRRAVELQTVAFPETPPPSYETVMAAAPPYVPPRYLGPTEGRNSIRYSELSPLYDTTRVYLVDNKSSDIASLNYQNDHSNFLTTVVQNNDYSPIEAGTQTINFDERSRWGGDLKTILHTNMPNVNDFMFTTKFKARVMVARKTNNEGQTILEYEWAEFVLPEGNYSETMTIDLMNNAIIEHYLRVGRQHGVLESDIGVKFDTRNFRLGWDPETQLVTPGVYTNEAFHPDIVLLPGCGVDFTESRLSNILGIRKRQPFQEGFVIMYEHLEGGNIPALLDVKKYENSLQDQNTVRGDNFIALNKAARIEPVETDPKGRSYNLLPDKKNTKYRSWYLAYNYGDPEKGVRSWTLLTTPDVTGGSEQVYWSLPDMMQDPVTFRSSRQVSNYPVVAAELLPVHAKSFYNEQAVYSQLIRQSTALTRVFNRFPENQILVRPPAATITTVSENVPALTDHGTLPLRSSISGVQRVTITDARRRTCPYVYKALGIVSPRVLSSRTF

SEQ ID NO: 8 - protein, 517 aa, Human adenovirus 17
MRRAVVSSSPPPSYESVMAQATLEVPFVPPRYMAPTEGRNSIRYSELAPLYDTTRVYLVDNKSADIASLNYQNDHSNFLTTVVQNNDFTPAEASTQTINFDERSRWGGDLKTILHTNMPNVNEYMFTSKFKARVMVARKHPQGVEATDLSKDILEYEWFEFTLPEGNFSETMTIDLMNNAILENYLQVGRQNGVLESDIGVKFDSRNFKLGWDPVTKLVMPGVYTYEAFHPDVVLLPGCGVDFTESRLSNLLGIRKKQPFQEGFRIMYEDLEGGNIPALLDVPKYLESKKKLEEALENAAKANGPARGDSSVSREVEKAAEKELVIEPIKQDDSKRSYNLIEGTMDTLYRSWYLSYTYGDPEKGVQSWTLLTTPDVTCGAEQVYWSLPDLMQDPVTFRSTQQVSNYPVVGAELMPFRAKSFYNDLAVYSQLIRSYTSLTHVFNRFPDNQILCRPPAPTITTVSENVPALTDHGTLPLRSSIRGVQRVTVTDARRRTCPYVYKALGIVAPRVLSSRTF

SEQ ID NO: 9 - protein, 520 aa, Human adenovirus 25
MRRAVVSSSPPPSYESVMAQATLEVPFVPPRYMAPTEGRNSIRYSELAPQYDTTRVYLVDNKSADIASLNYQNDHSNFLTTVVQNNDFTPAEASTQTINFDERSRWGGDLKTILHTNMPNVNEYMFTSKFKARVMVARKHPENVDKTDLSQDKLEYEWFEFTLPEGNFSETMTIDLMNNAILENYLQVGRQNGVLESDIGVKFDSRNFKLGWDPVTKLVMPGVYTYEAFHPDVVLLPGCGVDFTESRLSNLLGIRKKQPFQEGFRIMYEDLEGGNIPALLDTKKYLDSKKELEDAAKEAAKQQGDGAVTRGDTHLTVAQEKAAEKELVIVPIEKDESNRSYNLIKDTHDTMYRSWYLSYTYGDPEKGVQSWTLLTTPDVTCGAEQVYWSLPDLMQDPVTFRSTQQVSNYPVVGAELMPFRAKSFYNDLAVYSQLIRSYTSLTHVFNRFPDNQILCRPPAPTITTVSENVPALTDHGTLPLRSSIRGVQRVTVTDARRRTCPYVYKALGIVAPRVLSSRTF

SEQ ID NO: 10 - protein, 561 aa, Human adenovirus 35
MRRVVLGGAVVYPEGPPPSYESVMQQQQATAVMQSPLEAPFVPPRYLAPTEGRNSIRYSELAPQYDTTRLYLVDNKSADIASLNYQNDHSNFLTTVVQNNDFTPTEASTQTINFDERSRWGGQLKTIMHTNMPNVNEYMFSNKFKARVMVSRKPPDGAAVGDTYDHKQDILEYEWFEFTLPEGNFSVTMTIDLMNNAIIDNYLKVGRQNGVLESDIGVKFDTRNFKLGWDPETKLIMPGVYTYEAFHPDIVLLPGCGVDFTESRLSNLLGIRKKQPFQEGFKILYEDLEGGNIPALLDVDAYENSKKEQKAKIEAATAAAEAKANIVASDSTRVANAGEVRGDNFAPTPVPTAESLLADVSEGTDVKLTIQPVEKDSKNRSYNVLEDKINTAYRSWYLSYNYGDPEKGVRSWTLLTTSDVTCGAEQVYWSLPDMMKDPVTFRSTRQVSNYPVVGAELMPVFSKSFYNEQAVYSQQLRQSTSLTHVFNRFPENQILIRPPAPTITTVSENVPALTDHGTLPLRSSIRGVQRVTVTDARRRTCPYVYKALGIVAPRVLSSRTF

SEQ ID NO: 11 - protein, 519 aa, Human adenovirus 37
MRRAVVSSSPPPSYESVMAQATLEVPFVPPRYMAPTEGRNSIRYSELAPLYDTTRVYLVDNKSADIASLNYQNDHSNFLTTVVQNNDFTPAEASTQTINFDERSRWGGDLKTILHTNMPNVNEYMFTSKFKARVMVARKKAEGADANDRSKDILEYQWFEFTLPEGNFSETMTIDLMNNAILENYLQVGRQNGVLESDIGVKFDSRNFKLGWDPVTKLVMPGVYTYEAFHPDVVLLPGCGVDFTESRLSNLLGIRKKQPFQEGFRIMYEDLVGGNIPALLNVKEYLKDKEEAGKADANTIKAQNDAVPRGDNYASAAEAKAAGKEIELKAILKDDSDRSYNVIEGTTDTLYRSWYLSYTYGDPEKGVQSWTLLTTPDVTCGAEQVYWSLPDLMQDPVTFRSTQQVSNYPVVGAELMPFRAKSFYNDLAVYSQLIRSYTSLTHVFNRFPDNQILCRPPAPTITTVSENVPALTDHGTLPLRSSIRGVQRVTVTDARRRTCPYVYKALGIVAPRVLSSRTF

SEQ ID NO: 12 - protein, 508 aa, Human adenovirus 41
MRRAVGVPPVMAYAEGPPPSYESVMGSADSPATLEALYVPPRYLGPTEGRNSIRYSELAPLYDTTRVYLVDNKSADIASLNYQNDHSNFQTTVVQNNDFTPAEAGTQTINFDERSRWGADLKTILRTNMPNINEFMSTNKFKARLMVEKKNKETGLPRYEWFEFTLPEGNYSETMTIDLMNNAIVDNYLEVGRQNGVLESDIGVKFDTRNFRLGWDPVTKLVMPGVYTNEAFHPDIVLLPGCGVDFTQSRLSNLLGIRKRLPFQEGFQIMYEDLEGGNIPALLDVTKYEASIQKAKEEGKEIGDDTFATRPQDLVIEPVAKDSKNRSYNLLPNDQNNTAYRSWFLAYNYGDPNKGVQSWTLLTTADVTCGSQQVYWSLPDMMQDPVTFRPSTQVSNYPVVGVELLPVHAKSFYNEQAVYSQLIRQSTALTHVFNRFPENQILVRPPAPTITTVSENVPALTDHGTLPLRSSISGVQRVTITDARRRTCPYVHKALGIVAPKVLSSRTF

SEQ ID NO: 13 - protein, 575 aa, Gorilla adenovirus
MMRRAVLGGAVVYPEGPPPSYESVMQQQAAAVMQPSLEAPFVPPRYLAPTEGRNSIRYSELAPQYDTTRLYLVDNKSADIASLNYQNDHSNFLTTVVQNNDFTPTEASTQTINFDERSRWGGQLKTIMHTNMPNVNEYMFSNKFKARVMVSREASKIDSEKNDRSKDTLKYEWFEFTLPEGNFSATMTIDLMNNAIIDNYLAVGRQNGVLQSDIGVKFDTRNFRLGWDPVTKLVMPGVYTYEAFHPDIVLLPDCGVDFTESRLSNLLGIRKRHPFQEGFKIMYEDLEGGNIPALLDVAEYEKSKKEIASSTTTTAVTTVARNVADTSVEAVAVAVVDTIKAENDSAVRGDNFQSKNDMKASEEVTVVPVSPPTVTETETKEPTIKPLEKDTKDRSYNVISGTNDTAYRSWYLAYNYGDPEKGVRSWTLLTTSDVTCGAEQVYWSLPDMMQDPVTFRSTRQVSNYPVVGAELMPVFSKSFYNEQAVYSQQLRQTTSLTHIFDRFPENQILIRPPAPTITTVSENVPALTDHGTLPLRSSIRGVQRVTVTDARRRTCPYVYKALGIVAPRVLSSRTF

SEQ ID NO: 14 - protein, 532 aa, Chimpanzee adenovirus
MMRRAYPEGPPPSYESVMQQAMAAAAAMQPPLEAPYVPPRYLAPTEGRNSIRYSELAPLYDTTRLYLVDNKSADIASLNYQNDHSNFLTTVVQNNDFTPTEASTQTINFDERSRWGGQLKTIMHTNMPNVNEFMYSNKFKARVMVSRKTPNGVTVTDGSQDILEYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLEGGNIPALLDVDAYEKSKEESAAAATAAVATASTEVRGDNFASPAAVAAAEAAETESKIVIQPVEKDSKDRSYNVLPDKINTAYRSWYLAYNYGDPEKGVRSWTLLTTSDVTCGVEQVYWSLPDMMQDPVTFRSTRQVSNYPVVGAELLPVYSKSFFNEQAVYSQQLRAFTSLTHVFNRFPENQILVRPPAPTITTVSENVPALTDHGTLPLRSSIRGVQRVTVTDARRRTCPYVYKALGIVAPRVLSSRTF

SEQ ID NO: 15 - protein, 508 aa, Simian adenovirus 18
MRRAVGVPPVMAYAEGPPPSYETVMGAADSPATLEALYVPPRYLGPTEGRNSIRYSELAPLYDTTRVYLVDNKSADIASLNYQNDHSNFLTTVVQNNDFTPVEAGTQTINFDERSRWGGDLKTILRTNMPNINEFMSTNKFRARLMVEKVNKETNAPRYEWFEFTLPEGNYSETMTIDLMNNAIVDNYLEVGRQNGVLESDIGVKFDTRNFRLGWDPVTKLVMPGVYTNEAFHPDIVLLPGCGVDFTQSRLSNLLGIRKRMPFQAGFQIMYEDLEGGNIPALLDVAKYEASIQKAREQGQEIRGDNFTVIPRDVEIVPVEKDSKDRSYNLLPGDQTNTAYRSWFLAYNYGDPEKGVRSWTLLTTTDVTCGSQQVYWSLPDMMQDPVTFRPSSQVSNYPVVGVELLPVHAKSFYNEQAVYSQLIRQSTALTHVFNRFPENQILVRPPAPTITTVSENVPALTDHGTLPLRSSISGVQRVTITDARRRTCPYVHKALGIVAPKVLSSRTF

SEQ ID NO: 16 - protein, 512 aa, Simian adenovirus 20
MRRAVAIPSAAVALGPPPSYESVMASANLQAPLENPYVPPRYLEPTGGRNSIRYSELTPLYDTTRLYLVDNKSADIATLNYQNDHSNFLTSVVQNSDYTPAEASTQTINLDDRSRWGGDLKTILHTNMPNVNEFMFTNSFRAKLMVAHETNKDPVYKWVELTLPEGNFSETMTIDLMNNAIVDHYLAVGRQNGVKESEIGVKFDTRNFRLGWDPQTELVMPGVYTNEAFHPDVVLLPGCGVDFTYSRLSNLLGIRKRMPFQEGFQIMYEDLVGGNIPALLDVPAYEASITTVAAKEVRGDNFEAAAAAAATGAQPQAAPVVRPVTQDSKGRSYNIITGTNNTAYRSWYLAYNYGDPEKGVRSWTLLTTPDVTCGSEQVYWSMPDMYVDPVTFRSSQQVSSYPVVGAELLPIHSKSFYNEQAVYSQLIRQQTALTHVFNRFPENQILVRPPAPTITTVSENVPALTDHGTLPLQNSIRGVQRVTITDARRRTCPYVYKALGIVAPRVLSSRTF

SEQ ID NO: 17 - protein, 511 aa, Simian adenovirus 49
MRRAVPAAAIPATVAYADPPPSYESVMAGVPATLEAPYVPPRYLGPTEGRNSIRYSELAPLYDTTRVYLVDNKSADIASLNYQNDHSNFLTTVVQNNDFTPVEAGTQTINFDERSRWGGQLKTILHTNMPNVNEFMFTNSFRAKVMVSRKQNEEGQTELEYEWVEFVLPEGNYSETMTLDLMNNAIVDHYLLVGRQNGVLESDIGVKFDTRNFRLGWDPVTKLVMPGVYTNEAFHPDVVLLPGCGVDFTQSRLSNLLGIRKRQPFQEGFRIMYEDLEGGNIPALLNVKAYEDSIAAAMRKHNLPLRGDVFAVQPQEIVIQPVEKDGKERSYNLLPDDKNNTAYRSWYLAYNYGDPLKGVRSWTLLTTPDVTCGSEQVYWSLPDLMQDPVTFRPSSQVSNYPVVGAELLPLQAKSFYNEQAVYSQLIRQSTALTHVFNRFPENQILVRPPAATITTVSENVPALTDHGTLPLRSSISGVQRVTITDARRRTCPYVYKALGIVAPRVLSSRTF

SEQ ID NO: 18 - protein, 505 aa, Rhesus adenovirus 51
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRAKLMVEKSNAETRQPRYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVSKYEASIQRAKAEGREIRGDTFAVAPQDLEIVPLTKDSKDRSYNIINNTTDTLYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 19 - protein, 503 aa, Rhesus adenovirus 52
MRRAVRVTPAAYEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVKKVENQPPEYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPALLDVTKYEQSVQRAKAEGREIRGDTFAVSPQDLVIEPLEHDSKNRSYNLLPNKTDTAYRSWFLAYNYGDPEKGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 20 - protein, 504 aa, Rhesus adenovirus 53
MRRAVRVTPAVYAEGPPPSYESVMGSANVPATLEAPYVPPRYLGPTEGRNSIRYSELAPLYDTTKVYLVDNKSADIASLNYQNDHSNFLTTVVQNNDFTPTEAGTQTINFDERSRWGGQLKTILHTNMPNINEFMSTNKFRARLMVEKTSGQPPKYEWFEFTIPEGNYSETMTIDLMNNAIVDNYLQVGRQNGVLESDIGVKFDTRNFRLGWDPVTKLVMPGVYTNEAFHPDIVLLPGCGVDFTQSRLSNLLGIRKRRPFQEGFQIMYEDLEGGNIPGLLDVPAYEQSLQQAQEEGRVTRGDTFATAPNEVVIKPLLKDSKDRSYNIITDTTDTLYRSWFLAYNYGDPENGVRSWTILTTTDVTCGSQQVYWSLPDMMQDPVTFRPSTQVSNFPVVGTELLPVHAKSFYNEQAVYSQLIRQSTALTHVFNRFPENQILVRPPAPTITTVSENVPALTDHGTLPLRSSISGVQRVTITDARRRTCPYVYKALGVVAPKVLSSRTF

SEQ ID NO: 21 - protein, 20 aa, Artificial Sequence
  SITE 1..1: I or L
  Consensus sequence of floor region
  SITE 2..2: K or Q or E
  SITE 3..3
  SITE 4..4: L or V or I
  SITE 5..5: T or E or A or K or L
  SITE 6..6: E or K or T or Q
  SITE 8..8: S or P or D
  SITE 9..9: K or T or S
  SITE 10..10: K or S or N or G or D
  SITE 14..14: L or V
  SITE 15..15: I or L
  SITE 16..16: S or E or P
  SITE 17..17: no amino acid, or, if present, N
  SITE 18..18: D or G
  SITE 19..19: S or K or Q or T
  SITE 20..20: T or N or I or K or M
XXXXXXDXXXSYNXXXXXXX

SEQ ID NO: 22 - protein, 8 aa, Artificial Sequence
  Sequnece of fragment B
  SITE 5..5: D or N
THVFXRFP

SEQ ID NO: 23 - protein, 9 aa, Artificial Sequence
  SITE 1: D or E or N
  Consensus sequence of N-terminus of RGD loop region
  SITE 2: V or L or I
  SITE 3: A or D or E or K or S or T
  SITE 4: A or D or E or K
  SITE 5: F or Y or W
  SITE 6: A or D or E or N or Q
  SITE 7: A or D or E or N or K
  SITE 8: S or T
  SITE 9: any amino acid
XXXXXXXXX

SEQ ID NO: 24 - protein, 8 aa, Artificial Sequence
  SITE 1: any amino acid
  Consensus sequence of C-Terminus of RGD loop regioni
  SITE 2: I or L of V
  SITE 3: D or E or K or N or Q or V
  SITE 4: Cor G or P
  SITE 5: I or L or V
  SITE 6: D or E or S or T
  SITE 7: D or E or S ot T
  SITE 8: D or E
XXXXXXXX

SEQ ID NO: 25 - protein, 8 aa, Artificial Sequence
  SITE 1: F or Y or W
  Consensus sequence of N-terminus ov V loop
  SITE 2: H or K or R
  SITE 3: A or V or I or L
  SITE 4: H or K or R
  SITE 5: A or V or I or L
  SITE 6: A or V or I or L or M
  SITE 7: A or V or I or L
  SITE 8: any amino acid
XXXXXXXX

SEQ ID NO: 26 - protein, 7 aa, Artificial Sequence
  SITE 1: any amino acid
  Consensus sequence of C-terminus of V loop
  SITE 2: F or Y or W
  SITE 3: D or E or S or T
  SITE 4: F or Y or W
  SITE 5: A or F or V or Y or W
  SITE 6: D or E or S or T
  SITE 7: F or Y or W
XXXXXXX

SEQ ID NO: 27 - protein, 333 aa, Artificial Sequence
  ADDomer2_Head
MSYYHHHHHHDYDIPTTENLYFQGAMGSGIQPNVNEYMFSNKFKARVMVSRKAPEGVTVNDTYDHKEDILKYEWFEFILPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTKKELKIQPLEKDSKSRSYNVLEDKINTAYRSWYLSYNYGNPEKGIRSWTLLTTSDVTCGANGDSGNPVFSKSFYNEQAVYSQQLRQATSLTHVFNRFPENQILIRPPAPTITTVSENVP

SEQ ID NO: 28 - protein, 333 aa, Artificial Sequence
  ChimpCrown
MSYYHHHHHHDYDIPTTENLYFQGTIMHTNMPNVNEFMYSNKFKARVMVSRKAPEGVTVNDTYDHKEDILEYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLEGGNIPALLDVDAYEKSKKDTTTETTTKKELKIQPVEKDSKDRSYNVLPDKINTAYRSWYLAYNYGDPEKGVRSWTLLTTSDVTCGVEQAELLPVYSKSFFNEQAVYSQQLRAFTSLTHVFNRFPENQILVRPPAPTITTVSENVP

SEQ ID NO: 29 - protein, 8 aa, Artificial Sequence
  Linker
GAMGSGIQ

SEQ ID NO: 30 - protein, 8 aa, Artificial Sequence
  Linker
GANGDSGN

SEQ ID NO: 31 - DNA, 5664 bp, Artificial Sequence
  Vector pProEx containing coding sequence for ADDomer2_Head
gtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggtccgtataatctgtggaattgtgagcggataacaatttcacacaggaaacagaccatgtcgtactaccatcaccatcaccatcacgattacgatatcccaacgaccgaaaacctgtattttcagggcgccatgggatccggaattcaaccgaacgtgaacgaatatatgtttagcaacaaatttaaagcgcgcgtgatggtgagccgcaaagcgccggaaggcgtgaccgtgaacgatacctatgatcataaagaagatattctgaaatatgaatggtttgaatttattctgccggaaggcaactttagcgcgaccatgaccattgatctgatgaacaacgcgattattgataactatctggaaattggccgccagaacggcgtgctggaaagcgatattggcgtgaaatttgatacccgcaactttcgcctgggctgggatccggaaaccaaactgattatgccgggcgtgtatacctatgaagcgtttcatccggatattgtgctgctgccgggctgcggcgtggattttaccgaaagccgcctgagcaacctgctgggcattcgcaaacgccatccgtttcaggaaggctttaaaattatgtatgaagatctggaaggcggcaacattccggcgctgctggatgtgaccgcgtatgaagaaagcaaaaaagataccaccaccgaaaccaccaccaaaaaagaactgaaaattcagccgctggaaaaagatagcaaaagccgcagctataacgtgctggaagataaaattaacaccgcgtatcgcagctggtatctgagctataactatggcaacccggaaaaaggcattcgcagctggaccctgctgaccaccagcgatgtgacctgcggcgcgaacggcgatagcggcaacccggtgtttagcaaaagcttttataacgaacaggcggtgtatagccagcagctgcgccaggcgaccagcctgacccatgtgtttaaccgctttccggaaaaccagattctgattcgcccgccggcgccgaccattaccaccgtgagcgaaaacgtgccgtgataagcggccgctttcgaatctagagcctgcagtctcgaggcatgcggtaccaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctacagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgctatggtgcactctcagtacaatctgctctgatgccgcatagttaagccagtaccagtcacgtagcgatatcggagtgtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcacccaatacgcaaaccgcctctccccgcgcgttggccgattcattaatgcagctggcacgacaggtttcccgactggaaagcgggcagtgagcgcaacgcaattaatgtgagttagcgcgaattgatctg

SEQ ID NO: 32 - protein, 310 aa, Artificial Sequence
  Variant of ADDomer2_Head
GAMGSGIQPNVNEYMFSNKFKARVMVSRKAPEGVTVNDTYDHKEDILKYEWFEFILPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTKKELKIQPLEKDSKSRSYNVLEDKINTAYRSWYLSYNYGNPEKGIRSWTLLTTSDVTCGANGDSGNPVFSKSFYNEQAVYSQQLRQATSLTHVFNRFPENQILIRPPAPTITTVSENVP

SEQ ID NO: 33 - protein, 311 aa, Artificial Sequence
  Variant of ChimpCrown
QGTIMHTNMPNVNEFMYSNKFKARVMVSRKAPEGVTVNDTYDHKEDILEYEWVEFELPEGNFSVTMTIDLMNNAIIDNYLAVGRQNGVLESDIGVKFDTRNFRLGWDPVTELVMPGVYTNEAFHPDIVLLPGCGVDFTESRLSNLLGIRKRQPFQEGFQIMYEDLEGGNIPALLDVDAYEKSKKDTTTETTTKKELKIQPVEKDSKDRSYNVLPDKINTAYRSWYLAYNYGDPEKGVRSWTLLTTSDVTCGVEQAELLPVYSKSFFNEQAVYSQQLRAFTSLTHVFNRFPENQILVRPPAPTITTVSENVP

SEQ ID NO: 34 - protein, 144 aa, Artificial Sequence
  SITE 1..1: any or no amino acid
  Fragment D of general formula (III) or (IV)
  SITE 2..2: any or no amino acid
  SITE 3..3: any or no amino acid
  SITE 4..4: any or no amino acid
  SITE 5..5: any or no amino acid
  SITE 6..6: any or no amino acid
  SITE 7..7: any or no amino acid
  SITE 8..8: any or no amino acid
  SITE 9..9: any or no amino acid
  SITE 10..10: any or no amino acid
  SITE 11..11: any or no amino acid
  SITE 12..12: any or no amino acid
  SITE 13..13: any or no amino acid
  SITE 14..14: any or no amino acid
  SITE 15..15: any or no amino acid
  SITE 16..16: any or no amino acid
  SITE 17..17: any or no amino acid
  SITE 18..18: any or no amino acid
  SITE 19..19: any or no amino acid
  SITE 20..20: any or no amino acid
  SITE 21..21: any or no amino acid
  SITE 22..22: any or no amino acid
  SITE 23..23: any or no amino acid
  SITE 24..24: any or no amino acid
  SITE 25..25: any or no amino acid
  SITE 26..26: any or no amino acid
  SITE 27..27: any or no amino acid
  SITE 28..28: any or no amino acid
  SITE 29..29: any or no amino acid
  SITE 30..30: any or no amino acid
  SITE 31..31: any or no amino acid
  SITE 32..32: any or no amino acid
  SITE 33..33: any or no amino acid
  SITE 34..34: any or no amino acid
  SITE 35..35: any or no amino acid
  SITE 36..36: any or no amino acid
  SITE 37..37: any or no amino acid
  SITE 38..38: any or no amino acid
  SITE 39..39: any or no amino acid
  SITE 40..40: any or no amino acid
  SITE 41..41: any or no amino acid
  SITE 42..42: any or no amino acid
  SITE 43..43: any or no amino acid
  SITE 44..44: any or no amino acid
  SITE 45..45: any or no amino acid
  SITE 46..46: any or no amino acid
  SITE 47..47: any or no amino acid
  SITE 50..50: E or G
  SITE 59..59: E or S
  SITE 60..60: L or V
  SITE 61..61: A or S
  SITE 63..63: L or Q
  SITE 64..64: Y or E
  SITE 68..68: R or K
  SITE 69..69: V or L
  SITE 92..92: L or Q
  SITE 96..96: V or I
  SITE 101..101: F or Y
  SITE 102..102: T or S
  SITE 104...104: A or T or I or G
  SITE 107..107: S or G
  SITE 113..113: F or L
  SITE 115..115: E or D
  SITE 121..121: A or G
  SITE 122..122: D or Q
  SITE 127..127: L or M
  SITE 128..128: H or R
  REGION 129..144: Fragment D ends at position 129 or at a position from 130 to 144
  SITE 130..130: if present, N
  SITE 131..131: if present, M
  SITE 132..132: if present, P
  SITE 133..133: if present, N
  SITE 134..134: if present, V or I
  SITE 135..135: if present, N
  SITE 136..136: if present, E or D
  SITE 137..137: if present, Y or E
  SITE 138..138: if present, M
  SITE 139..139: if present, F or S or Y
  SITE 140..140: if present, T or S
  SITE 141..141: if present, S or N
  SITE 142..142: if present, K
  SITE 143..143: if present, F
  SITE 144..144: if present, K
XXXXXXXXXXXXXXXXXXXXXXXXXXXXXXXXXXXXXXXXXXXXXXXPTXGRNSIRYSXXXPXXDTTXXYLVDNKSADIASLNYQNDHSNFXTTVXQNNDXXPXEAXTQTINXDXRSRWGXXLKTIXXTXXXXXXXXXXXXXXX

SEQ ID NO: 35 - protein, 46 aa, Artificial Sequence
  SITE 1..1: if present, L or S
  REGION 1..12: Fragment E begins at a position from 1 to 11 or at position 12
  Fragment E of general formula (III) or IV)
  SITE 2..2: if present, T or P or C
  SITE 3..3: if preent, T or P
  SITE 4..4: if present, P or S or A or R
  SITE 5..5: if present, N or D
  SITE 6..6: if present, G or V
  SITE 7..7: if present, H or T
  SITE 8..8: if present, C
  SITE 9..9: if rpesent, G
  SITE 10..10: if present, A or V or S
  SITE 11..11: if present, E or Q
  SITE 20..20: L or M
  SITE 22..22: Q or K
  SITE 31..31: Q or R or S
  SITE 33..33: V or I
  SITE 34..34: S or N
  SITE 36..36: Y or F
  SITE 41..41: A or V
  SITE 44..44: if present, M or L
  SITE 45..45: if present, P
  SITE 46..46: if present, V or E
XXXXXXXXXXXQVYWSLPDXMXDPVTFRSTXQXXNXPVVGXELXXX

SEQ ID NO: 36 - protein, 53 aa, Artificial Sequence
  SITE 1..1: if, present N
  REGION 1..4: Fragment F begins at a position from 1 to 3 or at position 4
  Fragment F of general formula (III) or (IV)
  SITE 2..2: if present, V
  SITE 3..3: if present, P
  SITE 18..18: R or S or G
  SITE 25..25: V or I
  SITE 44..44: A or S
  SITE 46..46: R or K
XXXALTDHGTLPLRSSIXGVQRVTXTDARRRTCPYVYKALGIVXPXVLSSRTF

SEQ ID NO: 37 - protein, 4 aa, Artificial Sequence
  Linker Li
GGGS

SEQ ID NO: 38 - protein, 6 aa, Artificial Sequence
  Linker Li
GGSGGS

SEQ ID NO: 39 - protein, 22 aa, Artificial Sequence
  His-tag containing sequence
MSYYHHHHHHDYDIPTTENLYF

SEQ ID NO: 40 - protein, 18 aa, Chikungunya virus
  Major neutralizing epitope from Chikungunya virus
STKDNFNVYKATRPYLAH

SEQ ID NO: 41 - protein, 6 aa, Human adenovirus 3
HVFNRF

SEQ ID NO: 42 - protein, 345 aa, Artificial Sequence
  ADDobody containing the major neutralizing epitope from Chikungunya virus
MSYYHHHHHHDYDIPTTENLYFQGAMGSGIQPNVNEYMFSNKFKARVMVSRKAPEGVTVNDTYDHKEDILKYEWFEFILPEGNFSATMTIDLMNNAIIDNYLEIGRQNGVLESDIGVKFDTRNFRLGWDPETKLIMPGVYTYEAFHPDIVLLPGCGVDFTESRLSNLLGIRKRHPFQEGFKIMYEDLEGGNIPALLDVTAYEESKKDTTTETTTKKELKIQPLEKDSKSRSYNVLEDKINTAYRSWYLSYNYGNPEKGIRSWTLLTTSDVTCGANGDSGNPVFSKSFYNEQAVYSQQLRQATSLTSTKDNFNVYKATRPYLAHPENQILIRPPAPTITTVSENVP

(19) United States
(12) Patent Application Publication
(10) Pub. No.: US 2023/0101439 A1
(43) Pub. Date: Mar. 30, 2023

(54) Engineered Polypeptides Derived From Variable Domain of Adenovirus Penton Base

(71) Applicant: IMOPHORON LIMITED, Bristol (GB)

(72) Inventor: Frédéric Garzoni, Bristol (GB)

(21) Appl. No.: 17/797,941

(22) PCT Filed: Feb. 5, 2021
(86) PCT No.: PCT/EP2021/052884

§ 371 Date: Aug. 5, 2022

(30) Foreign Application Priority Data
Feb. 5, 2020 (EP) 20155742.8
Feb. 6, 2020 (EP) 20155982.0

(51) Int. Cl. C07K 14/005; C12N 15/10

(52) U.S. Cl. CPC C07K 14/005; C12N 15/1055; C12N 2710/10322; C12N 2710/10323; C12N 2710/10333; A61K 38/00

(57) ABSTRACT

An engineered polypeptide derived from adenovirus pentane base protein. The polypeptide of the invention is based on the “upper” alpha-helical domain of the adenovirus pentane base as shown in the pentane base atomic structure, but it lacks essentially completely any amino acids of the beta-barrel sheet domain showing a jellyroll fold structure (the jellyroll fold domain). The polypeptide contains at least the large fragment of the alpha-helical domain of the pentane base, which fragment includes the RGD loop(s) and the VLP loop, and may contain also the second, short fragment of the alpha-helical domain of the adenovirus pentane base. The polypeptide of the invention provides a new scaffold for optimized presentation of peptidic entities such as oligopeptides, polypeptide sequences, protein domains, proteins and protein complexes as high affinity agents to target molecules.

### FIELD OF THE INVENTION

The present invention relates to an engineered polypeptide derived from adenovirus pentane base protein. The polypeptide of the invention is based on the “upper” alpha-helical domain of the adenovirus pentane base as shown in the pentane base atomic structure, but it lacks essentially completely any amino acids of the beta-barrel sheet domain showing a jellyroll fold structure (the jellyroll fold domain). The polypeptide contains at least the large fragment (also referred to herein as the “big fragment”) of said alpha-helical domain of the pentane base, which fragment includes the RGD loop(s) and the VLP loop, and may contain also the second, short fragment of the alpha-helical domain of the adenovirus pentane base. The polypeptide of the invention provides a new scaffold for optimized presentation of peptidic entities such as oligopeptides, polypeptide sequences, protein domains, proteins and protein complexes made up of two, several or many subunits, preferably as high affinity agents to target molecules.

### REFERENCE TO SEQUENCE LISTING

This application contains a Sequence Listing. The application thus incorporates by reference the material in the ASCII text file 34U6691.txt, created on Aug. 1, 2022, and having a size of 125,422 bytes.

### BACKGROUND OF THE INVENTION

A prerequisite for successful protein scaffold design for presentation of peptidic entities such as oligopeptides, polypeptide sequences, protein domains, proteins and protein complexes, is a compact, stable protein domain which can accommodate modalities representing exposed and flexible loop structures that can accommodate said entities. In a preferred embodiment of the invention these displayed entities can represent any peptidic sequence which may be recognized by any binding partner for peptidic structures, e.g. a binder sequence and/or paratope sequence and/or sequences, for example, from a randomized library that can be presented to any chemical or biochemical, respectively, structure capable to be recognized by said binder sequence (such as those exemplified above), e.g. an antigen and/or a toxin and/or a venom and/or a chemical (FIG. 1). In addition, foreign sequences which may be inserted into one or more of the engineered polypeptide of the invention can represent antigens used for identification of specific binders, for example antibodies (FIG. 2).

Penton base proteins (protomers) from a number of Adenovirus (Ad) serotypes contain highly variable loop regions which can be functionalized for inserting foreign sequences encoding for oligopeptides, polypeptide sequences, protein domains, proteins and protein complexes as disclosed in WO2017/167988 A1. Adenovirus is one of the most commonly used gene therapy vector in humans. The adenovirus shell is predominantly built by two distinct proteins: the hexon protein, and the penton base protein, with the latter forming pentameric assemblies to which attaches the fiber protein characteristic for this virus. Penton base proteins of certain adenovirus serotypes were shown to spontaneously self-assemble into a multimeric superstructure when expressed recombinantly in absence of other adenoviral components. This superstructure represents a dodecahedron, formed by a total of 60 adenovirus base proteins arranged in twelve identical copies of the pentamer.

The technical problem underlying the invention is the provision of protein scaffolds for presentation of peptidic binding partners for target molecules.

The solution to the above technical problem is provided by the embodiments of the present invention as defined and disclosed in the claims, the present description and the accompanying drawings.

A close inspection of the high-resolution structure of the penton base protein (PDB ID 6HCR) evidenced that the penton base protein itself adopts a distinct two-domain architecture with one domain representing a beta-barrel jellyroll fold conjoined to a second domain stabilized by alpha-helices which in the present invention is referred to the “crown domain” (FIG. 3). The former mediates multimerization into the dodecahedron as evidenced by mutational studies, while the latter presents extended loops to the solvent on the dodecahedron surface. These loops are extremely variable in length and sequence content in different adenovirus serotypes, while the remainder of the base protein is highly conserved throughout the adenoviral species. The adenovirus dodecahedron represents a highly versatile display scaffold, for example for immunogenic peptides that can be inserted into the loops replacing naturally occurring sequences in the adenovirus penton base. Literally hundreds of heterologous peptides can thus be displayed efficiently on a single dodecahedron, if all insertion sites are occupied (see WO 2017/167988 A1). The dodecahedron can be produced recombinantly in very high amounts, it is exceptionally stable and can be stored at ambient temperature for indefinite time (cf. WO 2017/167988 A1). Exploiting these highly advantageous characteristics, synthetic dodecahedron-based particles displaying immunogenic peptides in their exposed loops can be engineered for potential use in a range of applications including (onco-)immunology and emerging infectious disease. Earlier, in the context of vaccine candidates, the present inventor had developed adenovirus dodecahedron into a synthetic BioBrick format facilitating epitope insertion into the exposed loops, and put in place an efficient adenovirus base protein production protocol based on the MultiBac platform as disclosed in WO 2017/167988 A1. More recently, when inspecting closely the structure of the adenovirus base protein, the present inventor recognized that the architecture represents a bona fide two-domain structure which may have arisen during evolution by gene fusion (FIG. 3). The two domains as it appeared could be easily split into two distinct compact entities: the beta-barrel domain (jellyroll fold domain) containing the multimerization information, and the alpha-helical domain resembling a crown.

Co-pending International Patent Application PCT/EP2019/070722 describes multimerizing polypeptides derived from the jellyroll fold domain of the penton base protein. While discovering that the penton base protein of adenovirus could be split into two domains, the inventor recognized that the alpha-helical crown domain itself is of great interest for adopting various non-adenoviral sequences as disclosed in WO 2017/167988 A1, and could be produced on its own.

The present invention provides engineered polypeptides consisting of or derived from, respectively, the adenovirus base-protein head domain (i.e. the penton base protein minus the multimerization domain), or specific fragments thereof, as an autonomous scaffold to form a separate, stable, highly versatile protein entity on its own. Because the highly variable loops in the crown domain are reminiscent of antibody complementarity determining regions (CDRs), the crown domain polypeptides according to the invention are also referred to as the “ADDobody” hereafter. The ADDobody polypeptide of the invention is capable of displaying multiple copies of any peptidic structure, in particular peptides, oligopeptides, protein domains, proteins or protein complexes. The ADDobody contains the large and small fragments of an adenovirus penton base alpha-helical domain. The invention also is directed to minimal ADDobodies (or miniADDobodies) containing only the large fragment of the alpha-helical domain.

These oligopeptides, polypeptide sequences, protein domains and proteins can include: (i) naturally occurring binder sequences or paratopes, (ii) binder sequences or paratopes obtained from random library and selection evolution (phage/ribosome display), (iii) antigenic entities that stimulate the immune system to trigger an immune response, for example for vaccination purposes, or for preparing antibodies or other binder molecules in cell culture, or in vitro in a test tube. Ideally such protein presenting such entities will be safe, non-immunogenic, efficient, and tunable. Moreover, they will be produced easily at industrial scale. In certain embodiments the polypeptide contains insertion sites which are within the VL-loop (also called V loop) and/or RGD loops as disclosed in WO 2017/167988 A1. According to the present invention, two more sites of flexibility for heterologous modification of the naturally sequences of existing adenovirus penton base proteins are disclosed which further adds to, e.g. flexible modification of the crown domain and the adenovirus penton base for including a multitude of possible heterologous peptidic structures.

In addition, the polypeptide of the invention can be engineered as a multivalent Virus Like Particle (VLP). The present disclosure describes the creation, design and production of the engineered polypeptide and its embodiment as a novel type of protein for presenting peptidic structures for presentation to target binding partners.

More particular, the present invention provides the following embodiments:

The invention provides an isolated engineered polypeptide comprising the amino acid stretches essentially corresponding to a first and a second fragment of the penton base wherein the first fragment of the polypeptide is present between the first and second amino acid stretches forming the jellyroll fold domain in the full length penton base and wherein the second fragment of the polypeptide is present between the second and third fragments forming the jellyroll fold domain in the full length penton base, respectively, wherein the isolated engineered domain lacks the amino acid stretches forming the jellyroll fold domain of the adenovirus penton base, wherein optionally the first and/or second fragments of the polypeptide contain(s) one or more heterologous modification(s).

Preferably, there is provided a polypeptide having the structure of the following general formula (I):

N-A-L-B-C (I)
wherein
A represents an amino acid stretch corresponding to the N-terminal amino acid stretch of the adenovirus penton base present between the first and the second amino acid stretch forming the jellyroll fold domain of the adenovirus penton base;
B represents an amino acid stretch corresponding to the C-terminal amino acid stretch of the adenovirus penton base inserted between the second and the third amino acid stretch forming the jellyroll fold domain of the adenovirus penton base;
L represents a chemical group selected from the group consisting of an amino acid, an oligopeptide and a polypeptide;
N may or may not be present, and, if present, represents a chemical group such as an amino acid, an oligopeptide and a polypeptide;
C may or may not be present, and, if present, represents a chemical group such as of an amino acid, an oligopeptide and a polypeptide;
wherein, optionally, fragment A and/or B contain(s) one or more heterologous modifications.

A preferred fragment or group, respectively, N comprises an amino acid sequence facilitating the purification of the polypeptide, e.g. a His tag. The same applies also to fragment or group, respectively, C.

More preferably, fragment A of the polypeptides as defined herein comprises an amino acid sequence selected from the group consisting of the amino acid sequences according to the following Table 1:

TABLE 1

| Sequence | | | N-terminal | C-terminal |
|---|---|---|---|---|
| based on | Sequence | SEQ | amino acid | amino acid |
| penton base | according to | ID | selected from | selected from |
| protomer of | UniProtAcc. No. | NO: | positions | positions |
| hAd3 | Q2Y0H9 | 1 | 130 to 137 | 399 to 405 |
| hAd2 | P03276 | 2 | 130 to 137 | 426 to 432 |
| hAd4 | Q2KSF3 | 3 | 126 to 133 | 380 to 386 |
| hAd5 | P12538 | 4 | 130 to 137 | 426 to 432 |
| hAd7 | Q9JFT6 | 5 | 130 to 137 | 399 to 405 |
| hAd11 | D2DM93 | 6 | 130 to 137 | 416 to 422 |
| hAd12 | P36716 | 7 | 120 to 127 | 352 to 358 |
| hAd17 | F1DT65 | 8 | 117 to 124 | 371 to 377 |
| hAd25 | M0QUK0 | 9 | 125 to 133 | 389 to 395 |
| hAd35 | Q7T941 | 10 | 131 to 138 | 446 to 452 |
| hAd37 | Q912J1 | 11 | 117 to 124 | 373 to 379 |
| hAd41 | F8WQN4 | 12 | 128 to 135 | 362 to 368 |
| gorAd | E5L3Q9 | 13 | 131 to 138 | 417 to 423 |
| ChimpAd | G9G849 | 14 | 126 to 133 | 373 to 379 |
| sAd18 | H8PFZ9 | 15 | 128 to 135 | 354 to 360 |
| sAd20 | F6KSU4 | 16 | 127 to 134 | 359 to 365 |
| sAd49 | F2WTK5 | 17 | 128 to 135 | 357 to 363 |
| rhAd51 | A0A0A1EWW1 | 18 | 125 to 132 | 353 to 359 |
| rhAd52 | A0A0A1EWX7 | 19 | 125 to 132 | 351 to 357 |
| rhAd53 | A0A0A1EWZ7 | 20 | 126 to 133 | 352 to 358 |

wherein, optionally, fragment A contains one or more heterologous modifications.

Preferably, fragment B of the polypeptides as defined herein comprises an amino acid sequence selected from the group consisting of the amino acid sequences according to the following Table 2:

TABLE 2

| Sequence | | | N-terminal | C-terminal |
|---|---|---|---|---|
| based on | Sequence | SEQ | amino acid | amino acid |
| penton base | according to | ID | selected from | selected from |
| protomer of | UniProtAcc. No. | NO: | positions | positions |
| hAd3 | Q2Y0H9 | 1 | 441 to 444 | 491 to 494 |
| hAd2 | P03276 | 2 | 468 to 471 | 518 to 521 |
| hAd4 | Q2KSF3 | 3 | 422 to 445 | 465 to 468 |
| hAd5 | P12538 | 4 | 468 to 471 | 491 to 494 |
| hAd7 | Q9JFT6 | 5 | 441 to 444 | 464 to 467 |
| hAd11 | D2DM93 | 6 | 458 to 461 | 481 to 484 |
| hAd12 | P36716 | 7 | 394 to 398 | 418 to 421 |
| hAd17 | F1DT65 | 8 | 414 to 417 | 438 to 441 |
| hAd25 | M0QUK0 | 9 | 441 to 444 | 454 to 457 |
| hAd35 | Q7T941 | 10 | 498 to 501 | 521 to 524 |
| hAd37 | Q912J1 | 11 | 415 to 418 | 438 to 441 |
| hAd41 | F8WQN4 | 12 | 405 to 408 | 438 to 441 |
| gorAd | E5L3Q9 | 13 | 459 to 462 | 482 to 485 |
| ChimpAd | G9G849 | 14 | 421 to 424 | 444 to 457 |
| sAd18 | H8PFZ9 | 15 | 396 to 399 | 419 to 422 |
| sAd20 | F6KSU4 | 16 | 401 to 404 | 424 to 427 |
| sAd49 | F2WTK5 | 17 | 399 to 402 | 422 to 425 |
| rhAd51 | A0A0A1EWW1 | 18 | 395 to 398 | 418 to 421 |
| rhAd52 | A0A0A1EWX7 | 19 | 393 to 396 | 416 to 419 |
| rhAd53 | A0A0A1EWZ7 | 20 | 394 to 397 | 417 to 420 |

wherein, optionally, fragment B contains one or more heterologous modifications.

In preferred embodiments of the polypeptides according to the invention fragment A and/or B contain(s) one or more heterologous modifications wherein said one or more heterologous modifications is/are contained in the following sites:
the RGD loop region of fragment A; and/or
the V loop (also referred to as “variable loop” of fragment A; and/or
the sequence (from N- to C-terminal)

(SEQ ID NO: 21)
X₁-X₂-X₃-X₄-X₅-X₆-D-X₇-X₈-X₉-S-Y-N-X₁₀-X₁₁-X₁₂-X₁₃-X₁₄-
X₁₅-X₁₆

(hereinafter referred to as the “floor region”) of fragment A, wherein
X₁ is I or L, and is preferably I;
X₂ is selected from the group consisting of K, Q and E, and is preferably Q;
X₃ is P or A, and is preferably P,
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably S,
X₈ is selected from the group consisting of K, T and S, and is preferably K;
X₉ is selected from the group consisting of K, S, N, G and D, and is preferably S,
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably ID,
X₁₅ is selected from the group consisting of S, K, Q and T, and is preferably K; and
X₁₆ is selected from the group consisting of T, N, I, K and M, and is preferably I; and/or
the sequence (from N- to C-terminal) T-H-V-F-X₁₇-R-F-P (SEQ ID NO: 22) (hereinafter referred to as the “B loop”) of fragment B wherein X₁₇ is D or N, and is preferably N.

According to the invention, it is surprisingly found that the floor region (also denoted as “floor site”) and the B loop show a flexible conformation as evidenced by X-ray chrystallography of an exemplary ADDobody of the invention.

It is to be understood that, with respect to the floor region (also referred to as “floor site”) and the B loop, which are both (more particularly the B loop) considerably conserved sites amongst the adenovirus penton base sequences of various adenovirus species, the one or more heterologous modification includes any insertion, deletion, replacement at any and of any, respectively, position of the residues outlined above, whereby the insertion or replacement may comprise one or more or all of the respective amino acids of the floor region and the B loop, respectively.

As regards the B loop, a preferred heterologous modification is a replacement of amino acid residues 1 to 6 of SEQ ID NO: 22 as defined above, preferably by a heterologous oligonucleotide, polypeptide, protein or protein complex.

In preferred embodiments of the invention, the polypeptide comprises one or more heterologous modifications at least in the RGD loop (i.e. the RGD loop region as defined above), the V loop and the floor site, wherein in certain embodiments of this type, the one or more heterologous modifications are located only in said RDG loop region, said V loop and said floor site. In other embodiments, the polypeptide of the invention comprises one or more heterologous modifications at least in the RGD loop region and the V loop, wherein in certain embodiments of this type, the one or more heterologous modifications are located only in said RDG loop region and said V loop. In other embodiments of the invention the polypeptide comprises one or more heterologous modifications at least in the floor region and the B loop, wherein in certain embodiments of this type, the one or more heterologous modifications are located only in said floor site and said B loop. It is understood that the one or more modifications may present in any combination of the sites for heterologous modification of fragment A and/or fragment B as defined above, including one or more heterologous modification in all of the above-defined sites.

More preferably, the N-terminus of the RGD loop region of fragment A is defined by the following sequence (from N-terminal to C-terminal):

(SEQ ID NO: 23)
X₁₈-X₁₉-X₂₀-X₂₁-X₂₂-X₂₃-X₂₄-X₂₅-X₂₆

wherein
X₁₈ is selected from the croup consisting of D, E and N, and is preferably D;
X₁₉ is selected from the group consisting of V, L, and I, and is preferably V;
X₂₀ is any amino acid, preferably selected from the group consisting of A, O, E, K, S, and T, and is more preferably T;
X₂₁ is any amino add, preferably selected from the group consisting of A, D, E, and K, and is more preferably A;
X₂₂ is selected from the group consisting of F, Y, and W, and is preferably Y;
X₂₃ is selected from the group consisting of A, D, E, N, and Q, is preferably E or Q, and is more preferably E;
X₂₄ is any amino acid, preferably selected from the group consisting of A, D, E, N, and K, and is more preferably E;
X₂₅ is selected from the group consisting of S or T, and is preferably S; and
X₂₆ is any amino acid and constitutes the N-terminal amino acid of the RGD loop region,

More preferably, the C-terminus of the RGD loop region of fragment A is defined by the following sequence (from N-terminal to C-terminal):

(SEQ ID NO: 24)
X₂₇-X₂₈-X₂₉-X₃₀-X₃₁-X₃₂-X₃₃-X₃₄

wherein
X₂₇ is any amino acid and constitutes the C-terminal amino acid of the second RGD loop;
X₂₈ is selected from the group consisting of I, L and V, and is preferably I;
X₂₉ is selected from the group consisting of D, E, K, N, O, and V, is preferably Q or K, and is more preferably Q;
X₃₀ is selected from the group consisting of C, G and P, and is preferably P;
X₃₁ is selected from the group consisting of i, L and V, is preferably L or V and is more preferably L;
X₃₂ is selected from the group consisting of D, E, S and T, is preferably E or T and is more preferably E;
X₃₃ is selected from the group consisting of D, E, S and T, is preferably E, or T, and is more preferably K; and
X₃₄ is selected from the group consisting of D and E, and is preferably D.

Referring to WO 2017/167988 A1, it is to be understood that, according to the invention, the RGD loop region as disclosed and defined herein can be sub-divided into a first and a second RDG loop, more particularly as defined on pages 31 to 33 of WO 2017/167988.

Preferably, the N-terminus of the V loop of fragment A is defined by the following sequence (from N-terminal to C-terminal):

(SEQ ID NO: 25)
X₃₅-X₃₆-X₃₇-X₃₈-X₃₉-X₄₀-X₄₁-X₄₂

wherein
X₃₅ is selected from the group consisting of F, Y, and W, and is preferably F;
X₃₆ is selected from the group consisting of H, K and R, and is preferably K;
X₃₇ is selected from the group consisting of A, V, I, and L, and is preferably A;
X₃₆ is selected from the group consisting of H, K, and R, and is preferably R;
X₃₉ is selected from the group consisting of A, V, I, and L, and is preferably V;
X₄₀ is selected from the group consisting of A, V, I, L and M, and is preferably M;
X₄₁ is selected from the group consisting of A, V, I, and L, and is preferably V; and
X₄₂ is any amino acid and constitutes the N-terminal amino acid of the V loop.

Preferably, the C-terminus of the V loop of fragment A is defined by the following sequence (from N-terminal to C-terminal):

(SEQ ID NO: 26)
X₄₃-X₄₄-X₄₅-X₄₆-X₄₇-X₄₈-X₄₉

wherein
X₄₃ is any amino acid and constitutes the C-terminal amino acid of the V loop;
X₄₄ is selected from the group consisting of F, Y, and IN, and is preferably Y;
X₄₅ is selected from the group consisting of D, E, S and T, is preferably E or T and is more preferably E;
X₄₆ is selected from the group consisting of F, Y, and W, and is preferably W;
X₄₇ is selected from the group consisting of A, F, V, Y, and VV, is preferably F or V and is more preferably F;
X₄₈ is selected from the group consisting of 0, E, S and T, is preferably D or E and is more preferably E; and
X₄₉ is selected from the group consisting of F, Y, and VV, and is preferably F.

According to another aspect, the present invention is also directed to a further isolated engineered polypeptide comprising the large fragment of the alpha-helical domain of an adenovirus penton base protein which polypeptide lacks the small fragment of the alpha-helical domain and the jellyroll fold domain of the adenovirus penton base protein, wherein said large fragment optionally contains one or more heterologous modifications. This further engineered polypeptide is referred to herein as “minimal crown domain” or “minimal ADDobody” or “miniADDobody”.

The minimal crown domain (or minimal ADDobody) of the invention has preferably a general structure as defined according to following formula (II):

N-A-C (II)

wherein N, A and —C are as defined above (formula (I).

The present invention also relates to a nucleic acid encoding a minimal ADDobody of the invention. The present invention further provides a vector comprising said nucleic acid encoding a minimal ADDobody, preferably said nucleic acid is contained in an expression cassette. There is further provided a recombinant host cell containing said minimal ADDobody vector. The invention further provides a method for producing a minimal ADDobody of the invention comprising the step of culturing the vector containing the minimal ADDobody coding sequence in an expression cassette under conditions allowing the expression of the minimal ADDobody, and optionally purifying the minimal ADDobody from the host cells.

A “heterologous modification” as defined herein may be any modification of the respective site (RGD loop region, V loop, floor site, B loop) compared to the respective naturally occurring sequence, preferably as found in the adenovirus penton base proteins as further described in more detail below. Preferably, the heterologous modification is selected from the group consisting of one or more single amino acid mutations in comparison to the wildtype sequence of fragment A and/or B, one or more replacements of wildtype amino acid stretches by one or more heterologous amino acids and/or amino acid stretches one or more insertions of heterologous amino acid stretches, one or more deletions of one or more amino acids and one or more amino acid modifications as well as any combination(s) thereof. Generally preferred modifications according to the invention are insertions in and/or replacements of amino acid residues of the wildtype adenoviral sequences of the sites as defined herein by non-adenoviral amino acid sequences, preferably non-adenoviral oligonucleotides, polypeptides, proteins and/or protein complexes.

A point mutation (there can be one or more, preferably in the respective sites of fragments A and/or B as defined herein) may, for example, the replacement of an amino acid by a coupling residue, i.e. a naturally or non-naturally occurring amino acid having a side chain capable of forming a covalent bond with a binding partner, for example another coupling residue present either on another polypeptide to be coupled to the coupling residue or in fragment A and/or B of the present invention and/or in the linker L and/or in fragment N and/or in fragment C of the polypeptide according to the invention. The latter may serve for stabilizing the structure of the ADDobody or minimal ADDobody, respectively, or by stabilizing dimers, decamers, pentamers and/or dodecahedrons of the polypeptides of the invention (for example, via head-to-tail, head-to-head or head-to-tail arrangement). Preferred coupling residues are amino acids D, E, K and C, with C being particularly preferred, since it may readily form a disulfide bond with another C under the appropriate redox conditions known in the art.

According to preferred embodiments the heterologous modification provides a target specific binding entity.

In preferred embodiments of the invention the target specific binding entity is selected from the group consisting of antigens, epitopes, CDRs, antibodies, antibody fragments such as an antigen binding (Fab) fragment, a Fab′ fragment, a F(ab′)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, a paratope, an alternative scaffold protein, and a fusion protein thereof.

According to the invention, a paratope, also called an antigen-binding site of an antibody, is a part of an antibody which recognizes and binds to an antigen, more particularly an epitope of an antigen. It is typically a short amino acid stretch, usually of 5 to 10 amino acids which are part of the Fab region of an antibody.

A specific polypeptide according to the invention has the following amino acid sequence (from N-terminal to C-terminal):

(SEQ ID NO: 27)
MSYYHHHHHHDYDIPTTENLYFQGAMGSGIQPNVNEYMFSNKFKARVMV
SRKAPEGVTVNDTYDHKEDILKYEWFEFILPEGNFSATMTIDLMNNAII
DNYLEIGRQNGVLESDIGVKFDTRNFRLGWDPETKLIMPGVYTYEAFHP
DIVLLPGCGVDFTESRLSNLLGIRKRHPFQEGFKIMYEDLEGGNIPALL
DVTAYEESKKDTTTETTTKKELKIQPLEKDSKSRSYNVLEDKINTAYRS
WYLSYNYGNPEKGIRSWTLLTTSDVTCGANGDSGNPVFSKSFYNEQAVY
SQQLRQATSLTHVFNRFPENQILIRPPAPTITTVSENVP

A variant of the above polypeptide of the invention according to SEQ ID NO: 27 has the following amino acid sequence (from N- to C-terminal):

(SEQ ID NO: 32)
GAMGSGIQPNVNEYMFSNKFKARVMVSRKAPEGVTVNDTYDHKEDILKY
EWFEFILPEGNFSATMTIDLMNNAIIDNYLEIGRQNGVLESDIGVKFDT
RNFRLGWDPETKLIMPGVYTYEAFHPDIVLLPGCGVDFTESRLSNLLGI
RKRHPFQEGFKIMYEDLEGGNIPALLDVTAYEESKKDTTTETTTKKELK
IQPLEKDSKSRSYNVLEDKINTAYRSWYLSYNYGNPEKGIRSWTLLTTS
DVTCGANGDSGNPVFSKSFYNEQAVYSQQLRQATSLTHVFNRFPENQIL
IRPPAPTITTVSENVP

A further specific polypeptide according to the invention has the following amino acid sequence (from N-terminal to C-terminal):

(SEQ ID NO: 28)
MSYYHHHHHHDYDIPTTENLYFQGTIMHTNMPNVNEFMYSNKFKARVMV
SRKAPEGVTVNDTYDHKEDILEYEWEFELPEGNFSVTMTIDLMNNAIID
NYLAVGRQNGVLESDIGVKFDTRNFRLGWDPVTELVMPGVYTNEAFHPD
IVLLPGCGVDFTESRLSNLLGIRKRQPFQEGFQIMYEDLEGGNIPALLD
VDAYEKSKKDTTTETTTKKELKIQPVEKDSKDRSYNVLPDKINTAYRSW
YLAYNYGDPEKGVRSWTLLTTSDVTCGVEQAELLPVYSKSFFNEQAVYS
QQLRAFTSLTHVFNRFPENQILVRPPAPTITTVSENVP

A variant of the above polypeptide of the invention according to SEQ ID NO: 27 has the following amino acid sequence (from N- to C-terminal):

(SEQ ID NO: 33)
QGTIMHTNMPNVNEFMYSNKFKARVMVSRKAPEGVTVNDTYDHKEDILE
YEVEFELPEGNFSVTMTIDLMNNAIIDNYLAVGRQNGVLESDIGVKFDT
RNFRLGWDPVTELVMPGVYTNEAFHPDIVLLPGCGVDFTESRLSNLLGI
RKRQPFQEGFQIMYEDLEGGNIPALLDVDAYEKSKKDTTTETTTKKELK
IQPVEKDSKDRSYNVLPDKINTAYRSWYLAYNYGDPEKGVRSWTLLTTS
DVTCGVEQAELLPVYSKSFFNEQAVYSQQLRAFTSLTHVFNRFPENQIL
VRPPAPTITTVSENVP

The invention is further directed to a nucleic acid encoding a polypeptide according to the invention.

There is also provided a vector comprising the nucleic acid of the invention (which is meant synonymous to a nucleotide sequence encoding a polypeptide of the invention). The vector may contain the nucleic acid (or the nucleotide sequence) within an expression cassette.

The invention also provides a recombinant host cell comprising the nucleic acid or the vector.

The invention furthermore is directed to a method for the production of a polypeptide according to the invention comprising the step of culturing the host cell containing the vector comprising the nucleic acid with an expression cassette under conditions allowing the expression of said polypeptide. The production method preferably comprises the step of purifying the polypeptide from the cultured host cells.

The invention also provides an engineered adenovirus penton base protein comprising a polypeptide of the invention (i.e. an ADDobody or a minimal ADDobody) comprising one or more heterologous modifications, preferably one or more heterologous modifications at least in the floor region and/or the B loop (with respect to engineered penton base proteins comprising an ADDobody) or preferably one or more heterologous modifications at least in the floor region (with respect to engineered penton base proteins comprising a minimal ADDobody), fused to the multimerization domain (jellyroll fold domain) of an adenovirus penton base protein. Preferably, a multimerization domain is selected from an adenovirus selected from of human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53).

Engineered penton base proteins of the invention comprising an ADDobody polypeptide of the invention have typically a structure according to the following formula (III) (from N- to C-terminal):

D-A-E-B-F (III)

wherein A and B are the fragments of the alpha-helical crown domain as defined above, and D, E and F are the amino acid sequences of an adenovirus penton base forming the multimerization (jellyroll fold) domain, wherein one or more heterologous modifications is/are present in the floor region of fragment A and/or in the B loop of fragment B.

Engineered penton base proteins of the invention comprising a minimal ADDobody polypeptide of the invention have typically a structure according to the following formula (IV) (from N- to C-terminal):

D-A-E-Li-F (IV)

wherein A is the large fragment of the alpha-helical crown domain as defined above and D, E and F are the amino acid sequences of an adenovirus penton base forming the multimerization (jellyroll fold) domain, wherein, optionally and preferably, one or more heterologous modifications is/are present in the floor region of fragment A, and wherein Li is a linker selected from peptides, oligopeptides, polypeptides, proteins and protein complexes. Preferred linkers as Li are selected from oligopeptide linkers such as oligopeptides having 4 to 10 amino acids, i.e. 4, 5, 6, 7, 8, 9, or 10 amino acids, preferably having amino acids G and S. A preferred example is GGGS (SEQ ID NO: 37). Another example is a linker composed of G and S and having multiple GGS repeats such as 2, 3, 4, 5 or more GGS repeats. A particularly preferred linker of this type is GGSGGS (SEQ ID NO: 38).

Preferred amino acid sequences forming the multimerization domain are taken from the adenovirus penton base sequences of SEQ ID Nos: 1 to 20.

More preferred amino acid sequences for fragments D, A and F are characterized as follows:

According to a preferred embodiment of the invention, amino acid stretch D of general formulae (III) and/or (IV) has the following consensus sequence (SEQ ID NO: 34):

U)₁₋₄₇ PTJ₁GRNSIRY SJ₂J₃x₄PJ₅J₆DTT J₇J₈YLVDNKSA DIASLNYQND HSNFJ₅TTVJ₉Q NNDJ₁₀J₁₁PJ₁₂EAJ₁₃ TQTINJ₁₄DJ₁₅RS RWGJ₁₆J₁₇LKTIJ₁₈ J₁₉TZ₁Z₂Z₃Z₄Z₅Z₆Z₇Z₈ Z₉Z₁₀Z₁₁Z₁₂Z₁₃Z₁₄Z₁₅
wherein: amino acid stretch D ends on the C-terminal side before Z₁ at residue T or at an amino acid from Z₁ to Z₁₅
   U is any or no amino acid
   J₁ is E or G
   J₂ is E or S
   J₃ is L or V
   J₄ is A or S
   J₅ is L or Q
   J₆ is Y or E
   J₇ is R or K
   J₈ is V or L
   J₉ is V or I
   J₁₀ is F or Y
   J₁₁ is T or S
   J₁₂ is A or T or I or G
   J₁₃ is S or G
   J₁₄ is F or L
   J₁₅ is E or D
   J₁₆ is A or G
   J₁₇ is D or Q
   J₁₈ is L or M
   J₁₉ is H or R
   Z₁ if present, is N
   Z₂ if present, is M
   Z₃ if present, is P
   Z₄ if present, is N
   Z₅ if present, is V or I
   Z₆ if present, is N
   Z₇ if present, is E or D
   Z₈ if present, is Y or F
   Z₉ if present, is M
   Z₁₀ if present, is F or S or Y
   Z₁₁ if present, is T or S
   Z₁₂ if present, is S or N
   Z₁₃ if present, is K
   Z₁₄ if present, is F
   Z₁₆ if present, is K

More preferred amino acid sequences of fragment D are outlined in the following Table 3:

TABLE 3
Preferred sequences for fragment D of general formulae (III) and (IV)

| Sequence | | | N-terminal | C-terminal |
|---|---|---|---|---|
| based on | Sequence | SEQ | amino acid | amino acid |
| penton base | according to | ID | selected from | selected from |
| protomer of | UniProtAcc. No. | NO: | positions | positions |
| hAd3 | Q2Y0H9 | 1 | 1 to 48 | 129 to 144 |
| hAd2 | PO3276 | 2 | 1 to 48 | 129 to 144 |
| hAd4 | Q2KSF3 | 3 | 1 to 44 | 125 to 140 |
| hAd5 | P12538 | 4 | 1 to 48 | 129 to 144 |
| hAd7 | Q9JFT6 | 5 | 1 to 48 | 129 to 144 |
| hAd11 | D2DM93 | 6 | 1 to 48 | 129 to 144 |
| hAd12 | P36716 | 7 | 1 to 38 | 119 to 134 |
| hAd17 | F1DT65 | 8 | 1 to 35 | 116 to 131 |
| hAd25 | M0QUK0 | 9 | 1 to 43 | 124 to 139 |
| hAd35 | Q7T941 | 10 | 1 to 49 | 130 to 145 |
| hAd37 | Q912J1 | 11 | 1 to 35 | 116 to 131 |
| hAd41 | F8WQN4 | 12 | 1 to 46 | 127 to 142 |
| gorAd | E5L3Q9 | 13 | 1 to 49 | 130 to 145 |
| ChimpAd | G9G849 | 14 | 1 to 44 | 125 to 140 |
| sAd18 | H8PFZ9 | 15 | 1 to 46 | 127 to 142 |
| sAd20 | F6KSU4 | 16 | 1 to 45 | 126 to 141 |
| sAd49 | F2WTK5 | 17 | 1 to 48 | 127 to 142 |
| rhAd51 | A0A0A1EWW1 | 18 | 1 to 43 | 124 to 139 |
| rhAd52 | A0A0A1EWX7 | 19 | 1to 43 | 124 to 139 |
| rhAd53 | A0A0A1 EWZ7 | 20 | 1 to 44 | 125 to 140 |

According to a further preferred embodiment of the invention, amino acid stretch E of above general formulae (III) and/or (IV) has the following sequence (SEQ ID NO: 35):

Z₁₇Z₁₈Z₁₉Z₂₀Z₂₁Z₂₂Z₂₃Z₂₄Z₂₅Z₂₈ Z₂₇QVYWSLPDJ₂₀ MJ₂₁DPVTFRST J₂₂QJ₂₃J₂₄NJ₂₆PVVGJ₂₆ ELZ₂₈Z₂₈Z₃₀

wherein: amino acid stretch E begins on the N-terminal side at an amino acid from Z₁₇ to Z₂₇ or at amino acid Q after Z₂₇,

amino acid stretch B ends on the C-terminal side before Z₂₈ at amino acid L or at an amino acid from Z₂₈ to Z₃₀;
Z₁₇ if present, is L or S
Z₁₈ if present, is T or P or C
Z₁₉ if present, is T or P
Z₂₀ if present, is P or S or A or R
Z₂₁ if present, is N or D
Z₂₂ if present, is G or V
Z₂₃ if present, is H or T
Z₂₄ if present, is C
Z₂₅ if present, is G
Z₂₈ if present, is A or V or S
Z₂₇, if present, is E or Q
J₂₀ is L or M
J₂₁ is Q or K
J₂₂ is Q or R or S
J₂₃ is V or I
J₂₄ is S or N
J₂₅ is Y or F
J₂₆ is A or V
Z₂₈, if present, is M or L
Z₂₉, if present, is P
Z₃₀, if present, is V or F

More preferred amino acid sequences of fragment E are outlined in the following Table 4:

TABLE 4
Preferred sequences for fragment E of general formulae (III) and (IV)

| Sequence | Sequence | | N-terminal | C-terminal |
|---|---|---|---|---|
| based on | according to | | amino acid | amino acid |
| penton base | UniProt | SEQ ID | selected from | selected from |
| protomer of | Acc. No. | NO: | positions | positions |
| hAd3 | Q2Y0H9 | 1 | 398 to 409 | 440 to 443 |
| hAd2 | P03276 | 2 | 425 to 436 | 467 to 470 |
| hAd4 | Q2KSF3 | 3 | 379 to 390 | 421 to 444 |
| hAd5 | P12538 | 4 | 425 to 436 | 467 to 470 |
| hAd7 | Q9JFT6 | 5 | 398 to 409 | 440 to 443 |
| hAd11 | D2DM93 | 6 | 415 to 426 | 457 to 460 |
| hAd12 | P36716 | 7 | 351 to 362 | 393 to 397 |
| hAd17 | F1DT65 | 8 | 370 to 381 | 413 to 416 |
| hAd25 | M0QUK0 | 9 | 388 to 399 | 440 to 443 |
| hAd35 | Q7T941 | 10 | 445 to 456 | 497 to 500 |
| hAd37 | Q912J1 | 11 | 372 to 383 | 414 to 417 |
| hAd41 | F8WQN4 | 12 | 362 to 373 | 404 to 407 |
| gorAd | E5L3Q9 | 13 | 416 to 427 | 458 to 461 |
| ChimpAd | G9G849 | 14 | 372 to 383 | 420 to 423 |
| sAd18 | H8PFZ9 | 15 | 353 to 364 | 395 to 398 |
| sAd20 | F6KSU4 | 16 | 358 to 369 | 400 to 403 |
| sAd49 | F2WTK5 | 17 | 356 to 367 | 398 to 401 |
| rhAd51 | A0A0A1EWW1 | 18 | 352 to 363 | 394 to 397 |
| rhAd52 | A0A0A1EWX7 | 19 | 350 to 361 | 392 to 395 |
| rhAd53 | A0A0A1EWZ7 | 20 | 351 to 362 | 393 to 396 |

According to a further preferred embodiment of the invention, fragment F of above general formulae (Ill) and/or (IV) has the following sequence (SEQ ID NO: 36):

Z₃₁Z₃₂Z₃₃ALTDHGT LPLRSSIJ₂₇GV QRVTJ₂₈TDARR RTCPYVYKA LGIVJ₃₀PJ₃₁VLS SRTF

wherein: amino acid stretch F begins on the N-terminal side at an amino acid from Z₃₁ to Z₃₃ or at amino acid A after Z₃₃;
Z₃₁, if present, is N
Z₃₂, if present, is V
Z₃₃, if present, is P
J₂₇ is R or S or G
J₂₈ is V or I
J₂₉ is Y or H
J₃₀ is A or S
J₃₁ is R or K

More preferred amino acid sequences of fragment F are outlined in the following Table 5:

TABLE 5
Preferred sequences for segment F of general formulae (III) and (IV)

| Sequence | | | N-terminal | |
|---|---|---|---|---|
| based on | Sequence | | amino acid | C-terminal |
| penton base | according to | SEQ ID | selected from | amino acid |
| protomer of | UniProt Acc. No. | NO: | positions | position |
| hAd3 | Q2Y0H9 | 1 | 492 to 495 | 544 |
| hAd2 | P03276 | 2 | 519 to 522 | 571 |
| hAd4 | Q2KSF3 | 3 | 466 to 469 | 535 |
| hAd5 | P12538 | 4 | 492 to 495 | 571 |
| hAd7 | Q9JFT6 | 5 | 465 to 468 | 544 |
| hAd11 | D2DM93 | 6 | 482 to 485 | 561 |
| hAd12 | P36716 | 7 | 419 to 422 | 497 |
| hAd17 | F1DT65 | 8 | 438 to 441 | 517 |
| hAd25 | M0QUK0 | 9 | 455 to 458 | 534 |
| hAd35 | Q7T941 | 10 | 522 to 525 | 561 |
| hAd37 | Q912J1 | 11 | 439 to 442 | 519 |
| hAd41 | F8WQN4 | 12 | 439 to 432 | 508 |
| gorAd | E5L3Q9 | 13 | 483 to 486 | 875 |
| ChimpAd | G9G849 | 14 | 445 to 458 | 532 |
| sAd18 | H8PFZ9 | 15 | 420 to 423 | 508 |
| sAd20 | F6KSU4 | 16 | 425 to 428 | 512 |
| sAd49 | F2WTK5 | 17 | 423 to 426 | 511 |
| rhAd51 | A0A0A1EWW1 | 18 | 419 to 422 | 505 |
| rhAd52 | A0A0A1EWX7 | 19 | 417 to 420 | 503 |
| rhAd53 | A0A0A1EWZ7 | 20 | 418 to 421 | 504 |

It is to be understood that the start and end amino acids of each of fragments A, B, D, E and F of the engineered adenovirus penton base of the invention according to general formula (III) are preferably selected such that there is preferably no overlap of the residues forming the transition from one fragment to the following fragment when compared to the sequences as shown in SEQ ID NOs: 1 to 20.

It is further to be understood that the start and end amino acids of each of fragments A, D, E and F of the engineered adenovirus penton base of the invention according to general formula (IV) are preferably selected such that there is preferably no overlap of the residues forming the transition from one fragment to the following fragment when compared to the sequences as shown in SEQ ID NOs: 1 to 20.

The fragments A, B, D, E and F of engineered penton base proteins of the invention according to formula (III) are preferably comprised of amino acid sequences of the same adenovirus serotype, but chimeras are contemplated as well.

The fragments A, D, E and F of engineered penton base proteins of the invention according to formula (IV) are preferably comprised of amino acid sequences of the same adenovirus serotype, but chimeras are contemplated as well.

The invention also provides pentameric complexes of an engineered adenovirus penton base protein of the invention, preferably a penton base protein of formula (III) or (IV), most preferably a penton base protein of formula (III).

The invention is further directed to virus-like particles (VLP) comprising 12 pentameric complexes of an engineered adenovirus penton base protein of the invention.

The invention also provides the use of polypeptides as defined herein having one or more heterologous modifications as outlined herein and/or of VLPs containing such polypeptides having one or more heterologous modifications as defined herein as a medicament.

The invention also provides pharmaceutical compositions comprising a polypeptide as defined herein having one or more heterologous modifications as outlined herein and/or a VLP containing such polypeptides having one or more heterologous modifications as defined herein, optionally together with at least one pharmaceutically acceptable carrier, excipient and/or diluent.

The invention further provides a method for producing a VLP as disclosed herein, preferably a VLP composed of polypeptides containing one or more heterologous modifications as defined herein, comprising the step of incubating a solution of a polypeptide according the invention, preferably a polypeptide comprising one or more heterologous modifications as defined herein under conditions allowing the assembly of the polypeptide into a VLP.

The invention also provides the use of polypeptides as defined herein having one or more heterologous modifications as outlined herein and/or of VLPs containing such polypeptides having one or more heterologous modifications as defined herein in the treatment and/or prevention of an infectious disease, an immune disease, tumor or cancer.

The invention is also directed to a method of identifying a binding sequence to a target molecule comprising the steps of:

(i) preparing a library of vectors each containing a nucleotide sequence encoding a polypeptide according to the invention having a candidate binding sequence in an expression cassette, each polypeptide encoded by said nucleotide sequence comprising a candidate binding sequence as a heterologous modification in one or more of the sites (RGD loop and/or V loop and/or floor region and/or B loop) as defined herein, wherein the candidate binding sequence encoded by the nucleotide sequence in each vector is different (i.e. the vectors preferably contain a randomized library of nucleotide sequences encoding randomized candidate binding sequences);
(ii) expressing the polypeptides encoded by the nucleotide sequences in a host cell or a cell-free system;
(iii) contacting the polypeptides expressed in step (ii), optionally after purification from the host cells or the cell-free system, with the target molecule; and (iv) detecting which polypeptide(s) have/has bound to the target molecule.

More particularly, above step (i) may be one of the following steps (ia) and (ib):

(ia) preparing a library of vectors each containing a nucleotide sequence encoding a polypeptide having a candidate binding sequence in an expression cassette, each polypeptide encoded by said nucleotide sequence comprising a candidate binding sequence as a heterologous modification in one or more of RGD loop region and/or V loop and/or floor region and/or B loop as defined in above, i.e. the nucleotide sequences in said library encode an ADDobody as defined herein, wherein the candidate binding sequence encoded by the nucleotide sequence in each vector is different such that the vectors contain a randomized library of nucleotide sequences encoding randomized candidate binding sequences; or
(ib) preparing a library of vectors each containing a nucleotide sequence encoding a polypeptide having a candidate binding sequence in an expression cassette, each polypeptide encoded by said nucleotide sequence comprising a candidate binding sequence as a heterologous modification in one or more of RGD loop region and/or V loop and/or floor region as defined above, i.e. the nucleotide sequences in said library encode a minimal ADDobody (also denoted as “miniADDobody”) as defined herein, wherein the candidate binding sequence encoded by the nucleotide sequence in each vector is different such that the vectors contain a randomized library of nucleotide sequences encoding randomized candidate binding sequences.

The method preferably further comprises the step of determining the dissociation constant(s) (Kd) of the polypeptide(s) bound to the target molecule.

The term “specific binding” as used in the context of the present invention to mean that a binding moiety (e.g. an antibody) binds stronger to a target, such as an epitope, for which it is specific compared to the binding to another target if it binds to the first target with a dissociation constant (Kd) which is lower than the dissociation constant for the second target. Targets can be recognized by their ligands which bind with a certain affinity to their targets and thus, the ligand binding to its respective target results in a biological effect. Preferably, the binding is both specific and occurs with a high affinity, preferably with a Kd of less than 10⁻⁷, 10⁻⁸, 10⁻⁹, 10⁻¹⁰ M or less. Such affinity is preferably measured at 37° C. Suitable assays include surface plasmon resonance measurements (e.g. Biacore), quartz crystal microbalance measurements (e.g. Attana), and competition assays.

As used herein, the term “Kd” (usually measured in “mol/L”, sometimes abbreviated as “M”) is intended to refer to the dissociation equilibrium constant of the particular interaction between a binding moiety (e.g. an antibody or fragment thereof) and a target molecule (e.g. an antigen or epitope thereof). Methods for determining Kd include, without limitation, ELISA and surface plasmon resonance assays.

The above identification method can also be provided as an evolutionary process wherein the identified sequences binding to the target are further optimized in subsequent rounds of library preparation (wherein the candidate sequences of each previous rounds are modified so as to provide improved binding to the target molecule), expression, and contacting with the target, optionally followed by determination of the dissociation constants so that improved binding candidates are selected in each subsequent rounds until a predetermined minimal dissociation constant is achieved, e.g. preferably a dissociation constant indicating a specific binding of the candidate sequence to the target, or until the dissociation constants are not further improved, typically not further lowered, in comparison to the previous round.

The nucleotide sequence encoding a thus selected and/or optimized, respectively, ADDobody (or minimal ADDobody) containing a, preferably the finally optimized, binding sequence can then genetically fused to the nucleic acid encoding a multimerization domain of the same or different, preferably the same, adenovirus penton base, for example by using appropriate restriction enzyme sites in the fragment encoding the optimized ADDobody or minimal ADDobody, respectively, and a vector containing the nucleotide sequence in an expression cassette. The nucleic acid encoding the, preferably optimized, ADDobody or minimal ADDobody, respectively, is than inserted in line with the nucleotide sequence encoding a multimerization domain so that a complete ADDobody-multimerization domain (or minimal ADDobody-multimerization domain) construct (i.e. an engineered penton base protein according to the invention) is generated within the expression cassette. The vector can then be introduced into appropriate host cells and the construct, also named an engineered adenovirus penton base, can be mass expressed and purified. VLPs can then be prepared from the purified engineered adenovirus penton bases which include multiple copies (up to 60 copies, if the binding sequence is present as a single copy per penton base in the VLP) of the selected/optimized binding sequence which leads, in terms of, for example, binding sequences directed to an antigen (or epitope thereof) to improved recognition of the target molecule. The system could be used, of course, for any binding partners, e.g. antigens or antibodies or fragments of such entities as further detailed herein.

The present invention also provides pentamers of the ADDobody of the invention. The present furthermore provides decamers of the ADDobody of the invention composed of two ADDobody pentamers.

It is to be understood that all engineered proteins and polypeptides disclosed herein can also be expressed from a corresponding expression vector in a cell-free expression system known in the art.

According to another aspect of the invention, there is provided an engineered penton base protein wherein said protein comprises a heterologous modification in the following sequence: (from N- to C-terminal)

(SEQ ID NO: 21)
X₁-X₂-X₃-X₄-X₅-X₆-D-X₇-X₈-X₉-S-Y-N-X₁₀-X₁₁-X₁₂-X₁₃-X₁₄-
X₁₅-X₁₆

of fragment A (also referred to as “floor site” or “floor region”), wherein
X₁ is I or L, and is preferably I;
X₂ is selected from the group consisting of K, Q and E, and is preferably Q;
X₃ is P or A, and is preferably P,
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably S;
X₈ is selected from the group consisting of K, T and S, and is preferably K;
X₉ is selected from the group consisting of K, S, N, G and D, and is preferably S;
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably ID,
X₁₅ is selected from the group consisting of S, K, Q and T, and is preferably K; and
X₁₆ is selected from the group consisting of T, N, I, K and M, and is preferably I;
and/or
in the following sequence (from N- to C-terminal) T-H-V-F-X₁₇-R-F-P (SEQ ID NO: 22) of fragment B (also referred to as “B loop) wherein X₁₇ is D or N, and is preferably N.

The linker L (formula (I)) according to the invention may be selected from oligopeptide linkers such as oligopeptides having 4 to 10 amino acids, i.e. 4, 5, 6, 7, 8, 9, or 10 amino acids. Larger oligopeptides of more than 10 amino acids, typically from 11 to 50 amino acids, are also contemplated. The linker L may also be a polypeptide, protein or protein complex provided that the linker L does not interfere with the proper folding and stability of the ADDobody. The same holds true for fragments N and C as defined herein. According to preferred embodiments of the invention, the linker L as defined herein may be selected from the amino acid sequences (from N- to C-terminal) GAMGSGIQ (SEQ ID NO: 29) and GANGDSGN (SEQ ID NO: 20).

As already outlined in the above embodiments of the invention (see Tables 1 and 2, the fragments A and/or B as well as the engineered adenonvirus penton base proteins of the invention are preferably based on an amino acid sequence which is each independently derived from penton base sequences selected from the group consisting of penton bases of human adenovirus serotype 2 (hAd2), human adenovirus serotype 3 (hAd3), human adenovirus serotype 4 (hAd4), human adenovirus serotype 5 (hAd5), human adenovirus serotype 7 (hAd7), human adenovirus serotype 11 (hAd11), human adenovirus serotype 12 (hAd12), human adenovirus serotype 17 (hAd17), human adenovirus serotype 25 (hAd25), human adenovirus serotype 35 (hAd35), human adenovirus serotype 37 (hAd37), human adenovirus serotype 41 (hAd41), gorilla adenovirus (gorAd), chimpanzee adenovirus (ChimpAd), simian adenovirus serotype 18 (sAd18), simian adenovirus serotype 20 (sAd20), simian adenovirus serotype 49 (sAd49), rhesus adenovirus serotype 51 (rhAd51), rhesus adenovirus serotype 52 (rhAd52), and rhesus adenovirus serotype 53 (rhAd53).

Preferred amino acid sequences of the above-indicated adenovirus penton bases are laid down in generally accessible databases such as UniProt and UniProtE, and especially preferred sequences referred to herein for the above-mentioned adenovirus subtypes are laid down in UniProt Acc. No. Q2YOH9 (human adenovirus serotype 3; SEQ ID NO: 1), UniProt Acc. No. P03276 (human adenovirus serotype 2; SEQ ID NO: 2), UniProt Acc. No. Q2KSF3 (human adenovirus serotype 4; SEQ ID NO: 3), UniProt Acc. No. P12538 (human adenovirus serotype 5; SEQ ID NO: 4), UniProt Acc. No. Q9JFT6 (human adenovirus serotype 7; SEQ ID NO: 5), UniProt Acc. No. D2DM93 (human adenovirus serotype 11; SEQ ID NO: 6), UniProt Acc. No. P36716 (human adenovirus serotype 12; SEQ ID NO: 7), UniProt Acc. No. F1DT65 (human adenovirus serotype 17; SEQ ID NO: 8), UniProt Acc. No. MOQUKO (human adenovirus serotype 25; SEQ ID NO: 9), UniProt Acc. No. Q7T941 (human adenovirus serotype 35; SEQ ID NO: 10), UniProt Acc. No. Q912J1 (human adenovirus serotype 37; SEQ ID NO: 11), UniProt Acc. No. F8WQN4 (human adenovirus serotype 41; SEQ ID NO: 12), UniProt Acc. No. E5L3Q9 (gorilla adenovirus; SEQ ID NO: 13), UniProt Acc. No. G9G849 (chimpanzee adenovirus; SEQ ID NO: 14), UniProt Acc. No. H8PFZ9 (simian adenovirus serotype 18; SEQ ID NO: 15), UniProt Acc. No. F6KSU4 (simian adenovirus serotype 20; SEQ ID NO: 16), UniProt Acc. No. F2VVTK5 (simian adenovirus serotype 49; SEQ ID NO: 17), UniProt Acc. No. A0A0A1EVWV1 (rhesus adenovirus serotype 51; SEQ ID NO: 18), UniProt Acc. No. A0A0A1EWX7 (rhesus adenovirus serotype 52; SEQ ID NO: 19), and UniProt Acc. No. A0A0A1 EWZ7 (rhesus adenovirus serotype 53; SEQ ID NO: 20).

The amino acid sequences of the above penton bases are as follows (the respective UniProt Acc. No. is indicated in brackets):

Human Adenovirus Serotype 3 penton base hAd3 (Q2Y0H9); SEQ ID NO: 1
MRRRAVLGGA VVYPEGPPPS YESVMQQQAA MIQPPLEAPF VPPRYLAPTE
GRNSIRYSEL SPLYDTTKLY LVDNKSADIA SLNYQNDHSN FLTTVVQNND
FTPTEASTQT INFDERSRWG GQLKTIMHTN MPNVNEYMFS NKFKARVMVS
RKAPEGVTVN DTYDHKEDIL KYEWFEFILP EGNFSATMTI DLMNNAIIDN
YLEIGRQNGV LESDIGVKFD TRNFRLGWDP ETKLIMPGVY TYEAFHPDIV
LLPGCGVDFT ESRLSNLLGI RKRHPFQEGF KIMYEDLEGG NIPALLDVTA
YEESKKDTTT ETTTLAVAEE TSEDDDITRG DTYITEKQKR EAAAAEVKKE
LKIQPLEKDS KSRSYNVLED KINTAYRSWY LSYNYGNPEK GIRSWTLLTT
SDVTCGAEQV YWSLPDMMQD PVTFRSTRQV NNYPVVGAEL MPVFSKSFYN
EQAVYSQQLR QATSLTHVFN RFPENQILIR PPAPTITTVS ENVPALTDHG
TLPLRSSIRG VQRVTVTDAR RRTCPYVYKA LGIVAPRVLS SRTF
hAd2 (P03276); SEQ ID NO: 2:
MQRAAMYEEG PPPSYESVVS AAPVAAALGS PFDAPLDPPF VPPRYLRPTG
GRNSIRYSEL APLFDTTRVY LVDNKSTDVA SLNYQNDHSN FLTTVIQNND
YSPGEASTQT INLDDRSHWG GDLKTILHTN MPNVNEFMFT NKFKARVMVS
RSLTKDKQVE LKYEWVEFTL PEGNYSETMT IDLMNNAIVE HYLKVGRQNG
VLESDIGVKF DTRNFRLGFD PVTGLVMPGV YTNEAFHPDI ILLPGCGVDF
THSRLSNLLG IRKRQPFQEG FRITYDDLEG GNIPALLDVD AYQASLKDDT
EQGGDGAGGG NNSGSGAEEN SNAAAAAMQP VEDMNDHAIR GDTFATRAEE
KRAEAEAAAE AAAPAAQPEV EKPQKKPVIK PLTEDSKKRS YNLISNDSTF
TQYRSWYLAY NYGDPQTGIR SWTLLCTPDV TCGSEQVYWS LPDMMQDPVT
FRSTSQISNF PVVGAELLPV HSKSFYNDQA VYSQLIRQFT SLTHVFNRFP
ENQILARPPA PTITTVSENV PALTDHGTLP LRNSIGGVQR VTITDARRRT
CPYVYKALGI VSPRVLSSRT F
hAd4 (Q2KSF3); SEQ ID NO: 3:
MMRRAYPEGP PPSYESVMQQ AMAAAAAIQP PLEAPYVPPR YLAPTEGRNS
IRYSELTPLY DTTRLYLVDN KSADIASLNY QNDHSNFLTT VVQNNDFTPT
EASTQTINFD ERSRWGGQLK TIMHTNMPNV NQFMYSNKFK ARVMVSRKTP
NGVTVGDNYD GSQDELKYEW VEFELPEGNF SVTMTIDLMN NAIIDNYLAV
GRQNGVLESD IGVKFDTRNF RLGWDPVTEL VMPGVYTNEA FHPDIVLLPG
CGVDFTESRL SNLLGIRKRQ PFQEGFQIMY EDLDGGNIPA LLDVEAYEKS
KEESVAAATT AVATASTEVR DDNFASAAAV AAVKADETKS KIVIQPVEKD
SKERSYNVLS DKKNTAYRSW YLAYNYGDRD KGVRSWTLLT TSDVTCGVEQ
VYWSLPDMMQ DPVTFRSTHQ VSNYPVVGAE LLPVYSKSFF NEQAVYSQQL
RAFTSLTHVF NRFPENQILV RPPAPTITTV SENVPALTDH GTLPLRSSIR
GVQRVTVTDA RRRTCPYVYK ALGIVAPRVL SSRTF
hAd5 (P12538); SEQ ID NO: 4:
MRRAAMYEEG PPPSYESVVS AAPVAAALGS PFDAPLDPPF VPPRYLRPTG
GRNSIRYSEL APLFDTTRVY LVDNKSTDVA SLNYQNDHSN FLTTVIQNND
YSPGEASTQT INLDDRSHWG GDLKTILHTN MPNVNEFMFT NKFKARVMVS
RLPTKDNQVE LKYEWVEFTL PEGNYSETMT IDLMNNAIVE HYLKVGRQNG
VLESDIGVKF DTRNFRLGFD PVTGLVMPGV YTNEAFHPDI ILLPGCGVDF
THSRLSNLLG IRKRQPFQEG FRITYDDLEG GNIPALLDVD AYQASLKDDT
EQGGGGAGGS NSSGSGAEEN SNAAAAAMQP VEDMNDHAIR GDTFATRAEE
KRAEAEAAAE AAAPAAQPEV EKPQKKPVIK PLTEDSKKRS YNLISNDSTF
TQYRSWYLAY NYGDPQTGIR SWTLLCTPDV TCGSEQVYWS LPDMMQDPVT
FRSTRQISNF PVVGAELLPV HSKSFYNDQA VYSQLIRQFT SLTHVFNRFP
ENQILARPPA PTITTVSENV PALTDHGTLP LRNSIGGVQR VTITDARRRT
CPYVYKALGI VSPRVLSSRT F
hAd7 (Q9JFT6); SEQ ID NO: 5:
MRRRAVLGGA MVYPEGPPPS YESVMQQQAA MIQPPLEAPF VPPRYLAPTE
GRNSIRYSEL SPLYDTTKLY LVDNKSADIA SLNYQNDHSN FLTTVVQNND
FTPTEASTQT INFDERSRWG GQLKTIMHTN MPNVNEYMFS NKFKARVMVS
RKAPEGVIVN DTYDHKEDIL KYEWFEFTLP EGNFSATMTI DLMNNAIIDN
YLEIGRQNGV LESDIGVKFD TRNFRLGWDP ETKLIMPGVY TYEAFHPDIV
LLPGCGVDFT ESRLSNLLGI RKRHPFQEGF KIMYEDLEGG NIPALLDVTA
YEESKKDTTT ETTTLAVAEE TSEDDNITRG DTYITEKQKR EAAAAEVKKE
LKIQPLEKDS KSRSYNVLED KINTAYRSWY LSYNYGNPEK GIRSWTLLTT
SDVTCGAEQV YWSLPDMMQD PVTFRSTRQV NNYPVVGAEL MPVFSKSFYN
EQAVYSQQLR QATSLTHVFN RFPENQILIR PPAPTITTVS ENVPALTDHG
TLPLRSSIRG VQRVTVTDAR RRTCPYVYKA LGIVAPRVLS SRTF
hAd11 (D2DM93); SEQ ID NO: 6:
MRRVVLGGAV VYPEGPPPSY ESVMQQQATA VMQSPLEAPF VPPRYLAPTE
GRNSIRYSEL APQYDTTRLY LVDNKSADIA SLNYQNDHSN FLTTVVQNND
FTPTEASTQT INFDERSRWG GQLKTIMHTN MPNVNEYMFS NNFKARVMVS
RKPPEGAAVG DTYDHKQDIL EYEWFEFTLP EGNFSVTMTI DLMNNAIIDN
YLKVGRQNGV LESDIGVKFD TRNFKLGWDP ETKLIMPGVY TYEAFHPDIV
LLPGCGVDFT ESRLSNLLGI RKKQPFQEGF KILYEDLEGG NIPALLDVDA
YENSKKEQKA KIEAAAEAKA NIVASDSTRV ANAGEVRGDN FAPTPVPTAE
SLLADVSGGT DVKLTIQPVE KDSKNRSYNV LEDKINTAYR SWYLSYNYGD
PEKGVRSWTL LTTSDVTCGA EQVYWSLPDM MQDPVTFRST RQVSNYPVVG
AELMPVFSKS FYNEQAVYSQ QLRQSTSLTH VFNRFPENQI LIRPPAPTIT
TVSENVPALT DHGTLPLRSS IRGVQRVTVT DARRRTCPYV YKALGIVAPR
VLSSRTF
hAd12 (P36716); SEQ ID NO: 7:
MRRAVELQTV AFPETPPPSY ETVMAAAPPY VPPRYLGPTE GRNSIRYSEL
SPLYDTTRVY LVDNKSSDIA SLNYQNDHSN FLTTVVQNND YSPIEAGTQT
INFDERSRWG GDLKTILHTN MPNVNDFMFT TKFKARVMVA RKTNNEGQTI
LEYEWAEFVL PEGNYSETMT IDLMNNAIIE HYLRVGRQHG VLESDIGVKF
DTRNFRLGWD PETQLVTPGV YTNEAFHPDI VLLPGCGVDF TESRLSNILG
IRKRQPFQEG FVIMYEHLEG GNIPALLDVK KYENSLQDQN TVRGDNFIAL
NKAARIEPVE TDPKGRSYNL LPDKKNTKYR SWYLAYNYGD PEKGVRSWTL
LTTPDVTGGS EQVYWSLPDM MQDPVTFRSS RQVSNYPVVA AELLPVHAKS
FYNEQAVYSQ LIRQSTALTR VFNRFPENQI LVRPPAATIT TVSENVPALT
DHGTLPLRSS ISGVQRVTIT DARRRTCPYV YKALGIVSPR VLSSRTF
hAd17 (F1DT65); SEQ ID NO: 8:
MRRAVVSSSP PPSYESVMAQ ATLEVPFVPP RYMAPTEGRN SIRYSELAPL
YDTTRVYLVD NKSADIASLN YQNDHSNFLT TVVQNNDFTP AEASTQTINF
DERSRWGGDL KTILHTNMPN VNEYMFTSKF KARVMVARKH PQGVEATDLS
KDILEYEWFE FTLPEGNFSE TMTIDLMNNA ILENYLQVGR QNGVLESDIG
VKFDSRNFKL GWDPVTKLVM PGVYTYEAFH PDVVLLPGCG VDFTESRLSN
LLGIRKKQPF QEGFRIMYED LEGGNIPALL DVPKYLESKK KLEEALENAA
KANGPARGDS SVSREVEKAA EKELVIEPIK QDDSKRSYNL IEGTMDTLYR
SWYLSYTYGD PEKGVQSWTL LTTPDVTCGA EQVYWSLPDL MQDPVTFRST
QQVSNYPVVG AELMPFRAKS FYNDLAVYSQ LIRSYTSLTH VFNRFPDNQI
LCRPPAPTIT TVSENVPALT DHGTLPLRSS IRGVQRVTVT DARRRTCPYV
YKALGIVAPR VLSSRTF
hAd25 (M0QUK0); SEQ ID NO: 9:
MRRAVVSSSP PPSYESVMAQ ATLEVPFVPP RYMAPTEGRN SIRYSELAPQ
YDTTRVYLVD NKSADIASLN YQNDHSNFLT TVVQNNDFTP AEASTQTINF
DERSRWGGDL KTILHTNMPN VNEYMFTSKF KARVMVARKH PENVDKTDLS
QDKLEYEWFE FTLPEGNFSE TMTIDLMNNA ILENYLQVGR QNGVLESDIG
VKFDSRNFKL GWDPVTKLVM PGVYTYEAFH PDVVLLPGCG VDFTESRLSN
LLGIRKKQPF QEGFRIMYED LEGGNIPALL DTKKYLDSKK ELEDAAKEAA
KQQGDGAVTR GDTHLTVAQE KAAEKELVIV PIEKDESNRS YNLIKDTHDT
MYRSWYLSYT YGDPEKGVQS WTLLTTPDVT CGAEQVYWSL PDLMQDPVTF
RSTQQVSNYP VVGAELMPFR AKSFYNDLAV YSQLIRSYTS LTHVFNRFPD
NQILCRPPAP TITTVSENVP ALTDHGTLPL RSSIRGVQRV TVTDARRRTC
PYVYKALGIV APRVLSSRTF
hAd35 (Q7T941); SEQ ID NO: 10:
MRRVVLGGAV VYPEGPPPSY ESVMQQQQAT AVMQSPLEAP FVPPRYLAPT
EGRNSIRYSE LAPQYDTTRL YLVDNKSADI ASLNYQNDHS NFLTTVVQNN
DFTPTEASTQ TINFDERSRW GGQLKTIMHT NMPNVNEYMF SNKFKARVMV
SRKPPDGAAV GDTYDHKQDI LEYEWFEFTL PEGNFSVTMT IDLMNNAIID
NYLKVGRQNG VLESDIGVKF DTRNFKLGWD PETKLIMPGV YTYEAFHPDI
VLLPGCGVDF TESRLSNLLG IRKKQPFQEG FKILYEDLEG GNIPALLDVD
AYENSKKEQK AKIEAATAAA EAKANIVASD STRVANAGEV RGDNFAPTPV
PTAESLLADV SEGTDVKLTI QPVEKDSKNR SYNVLEDKIN TAYRSWYLSY
NYGDPEKGVR SWTLLTTSDV TCGAEQVYWS LPDMMKDPVT FRSTRQVSNY
PVVGAELMPV FSKSFYNEQA VYSQQLRQST SLTHVFNRFP ENQILIRPPA
PTITTVSENV PALTDHGTLP LRSSIRGVQR VTVTDARRRT CPYVYKALGI
VAPRVLSSRT F
hAd37 (Q912J1); SEQ ID NO: 11
MRRAVVSSSP PPSYESVMAQ ATLEVPFVPP RYMAPTEGRN SIRYSELAPL
YDTTRVYLVD NKSADIASLN YQNDHSNFLT TVVQNNDFTP AEASTQTINF
DERSRWGGDL KTILHTNMPN VNEYMFTSKF KARVMVARKK AEGADANDRS
KDILEYQWFE FTLPEGNFSE TMTIDLMNNA ILENYLQVGR QNGVLESDIG
VKFDSRNFKL GWDPVTKLVM PGVYTYEAFH PDVVLLPGCG VDFTESRLSN
LLGIRKKQPF QEGFRIMYED LVGGNIPALL NVKEYLKDKE EAGKADANTI
KAQNDAVPRG DNYASAAEAK AAGKEIELKA ILKDDSDRSY NVIEGTTDTL
YRSWYLSYTY GDPEKGVQSW TLLTTPDVTC GAEQVYWSLP DLMQDPVTFR
STQQVSNYPV VGAELMPFRA KSFYNDLAVY SQLIRSYTSL THVFNRFPDN
QILCRPPAPT ITTVSENVPA LTDHGTLPLR SSIRGVQRVT VTDARRRTCP
YVYKALGIVA PRVLSSRTF
hAd41 (F8WQN4); SEQ ID NO: 12:
MRRAVGVPPV MAYAEGPPPS YESVMGSADS PATLEALYVP PRYLGPTEGR
NSIRYSELAP LYDTTRVYLV DNKSADIASL NYQNDHSNFQ TTVVQNNDFT
PAEAGTQTIN FDERSRWGAD LKTILRTNMP NINEFMSTNK FKARLMVEKK
NKETGLPRYE WFEFTLPEGN YSETMTIDLM NNAIVDNYLE VGRQNGVLES
DIGVKFDTRN FRLGWDPVTK LVMPGVYTNE AFHPDIVLLP GCGVDFTQSR
LSNLLGIRKR LPFQEGFQIM YEDLEGGNIP ALLDVTKYEA SIQKAKEEGK
EIGDDTFATR PQDLVIEPVA KDSKNRSYNL LPNDQNNTAY RSWFLAYNYG
DPNKGVQSWT LLTTADVTCG SQQVYWSLPD MMQDPVTFRP STQVSNYPVV
GVELLPVHAK SFYNEQAVYS QLIRQSTALT HVFNRFPENQ ILVRPPAPTI
TTVSENVPAL TDHGTLPLRS SISGVQRVTI TDARRRTCPY VHKALGIVAP
KVLSSRTF
Gorilla Adenovirus Penton Base gorAd (E5L3Q9); SEQ ID NO: 13:
MMRRAVLGGA VVYPEGPPPS YESVMQQQAA AVMQPSLEAP FVPPRYLAPT
EGRNSIRYSE LAPQYDTTRL YLVDNKSADI ASLNYQNDHS NFLTTVVQNN
DFTPTEASTQ TINFDERSRW GGQLKTIMHT NMPNVNEYMF SNKFKARVMV
SREASKIDSE KNDRSKDTLK YEWFEFTLPE GNFSATMTID LMNNAIIDNY
LAVGRQNGVL QSDIGVKFDT RNFRLGWDPV TKLVMPGVYT YEAFHPDIVL
LPDCGVDFTE SRLSNLLGIR KRHPFQEGFK IMYEDLEGGN IPALLDVAEY
EKSKKEIASS TTTTAVTTVA RNVADTSVEA VAVAVVDTIK AENDSAVRGD
NFQSKNDMKA SEEVTVVPVS PPTVTETETK EPTIKPLEKD TKDRSYNVIS
GTNDTAYRSW YLAYNYGDPE KGVRSWTLLT TSDVTCGAEQ VYWSLPDMMQ
DPVTFRSTRQ VSNYPVVGAE LMPVFSKSFY NEQAVYSQQL RQTTSLTHIF
DRFPENQILI RPPAPTITTV SENVPALTDH GTLPLRSSIR GVQRVTVTDA
RRRTCPYVYK ALGIVAPRVL SSRTF
Cimpanzee Adenovirus Penton Base chimpAd (G9G849); SEQ ID NO: 14:
MMRRAYPEGP PPSYESVMQQ AMAAAAAMQP PLEAPYVPPR YLAPTEGRNS
IRYSELAPLY DTTRLYLVDN KSADIASLNY QNDHSNFLTT VVQNNDFTPT
EASTQTINFD ERSRWGGQLK TIMHTNMPNV NEFMYSNKFK ARVMVSRKTP
NGVTVTDGSQ DILEYEWVEF ELPEGNFSVT MTIDLMNNAI IDNYLAVGRQ
NGVLESDIGV KFDTRNFRLG WDPVTELVMP GVYTNEAFHP DIVLLPGCGV
DFTESRLSNL LGIRKRQPFQ EGFQIMYEDL EGGNIPALLD VDAYEKSKEE
SAAAATAAVA TASTEVRGDN FASPAAVAAA EAAETESKIV IQPVEKDSKD
RSYNVLPDKI NTAYRSWYLA YNYGDPEKGV RSWTLLTTSD VTCGVEQVYW
SLPDMMQDPV TFRSTRQVSN YPVVGAELLP VYSKSFFNEQ AVYSQQLRAF
TSLTHVFNRF PENQILVRPP APTITTVSEN VPALTDHGTL PLRSSIRGVQ
RVTVTDARRR TCPYVYKALG IVAPRVLSSR TF
Simian Adenovirus Serotype 18 Penton Base, sAd18 (H8PFZ9); SEQ ID NO: 15
MRRAVGVPPV MAYAEGPPPS YETVMGAADS PATLEALYVP PRYLGPTEGR
NSIRYSELAP LYDTTRVYLV DNKSADIASL NYQNDHSNFL TTVVQNNDFT
PVEAGTQTIN FDERSRWGGD LKTILRTNMP NINEFMSTNK FRARLMVEKV
NKETNAPRYE WFEFTLPEGN YSETMTIDLM NNAIVDNYLE VGRQNGVLES
DIGVKFDTRN FRLGWDPVTK LVMPGVYTNE AFHPDIVLLP GCGVDFTQSR
LSNLLGIRKR MPFQAGFQIM YEDLEGGNIP ALLDVAKYEA SIQKAREQGQ
EIRGDNFTVI PRDVEIVPVE KDSKDRSYNL LPGDQTNTAY RSWFLAYNYG
DPEKGVRSWT LLTTTDVTCG SQQVYWSLPD MMQDPVTFRP SSQVSNYPVV
GVELLPVHAK SFYNEQAVYS QLIRQSTALT HVFNRFPENQ ILVRPPAPTI
TTVSENVPAL TDHGTLPLRS SISGVQRVTI TDARRRTCPY VHKALGIVAP
KVLSSRTE
sAd20 (F6KSU4); SEQ ID NO: 16:
MRRAVAIPSA AVALGPPPSY ESVMASANLQ APLENPYVPP RYLEPTGGRN
SIRYSELTPL YDTTRLYLVD NKSADIATLN YQNDHSNFLT SVVQNSDYTP
AEASTQTINL DDRSRWGGDL KTILHTNMPN VNEFMFTNSF RAKLMVAHET
NKDPVYKWVE LTLPEGNFSE TMTIDLMNNA IVDHYLAVGR QNGVKESEIG
VKFDTRNFRL GWDPQTELVM PGVYTNEAFH PDVVLLPGCG VDFTYSRLSN
LLGIRKRMPF QEGFQIMYED LVGGNIPALL DVPAYEASIT TVAAKEVRGD
NFEAAAAAAA TGAQPQAAPV VRPVTQDSKG RSYNIITGTN NTAYRSWYLA
YNYGDPEKGV RSWTLLTTPD VTCGSEQVYW SMPDMYVDPV TFRSSQQVSS
YPVVGAELLP IHSKSFYNEQ AVYSQLIRQQ TALTHVFNRF PENQILVRPP
APTITTVSEN VPALTDHGTL PLQNSIRGVQ RVTITDARRR TCPYVYKALG
IVAPRVLSSR TF
sAd49 (F2WTK5); SEQ ID NO: 17:
MRRAVPAAAI PATVAYADPP PSYESVMAGV PATLEAPYVP PRYLGPTEGR
NSIRYSELAP LYDTTRVYLV DNKSADIASL NYQNDHSNFL TTVVQNNDFT
PVEAGTQTIN FDERSRWGGQ LKTILHTNMP NVNEFMFTNS FRAKVMVSRK
QNEEGQTELE YEWVEFVLPE GNYSETMTLD LMNNAIVDHY LLVGRQNGVL
ESDIGVKFDT RNFRLGWDPV TKLVMPGVYT NEAFHPDVVL LPGCGVDFTQ
SRLSNLLGIR KRQPFQEGFR IMYEDLEGGN IPALLNVKAY EDSIAAAMRK
HNLPLRGDVF AVQPQEIVIQ PVEKDGKERS YNLLPDDKNN TAYRSWYLAY
NYGDPLKGVR SWTLLTTPDV TCGSEQVYWS LPDLMQDPVT FRPSSQVSNY
PVVGAELLPL QAKSFYNEQA VYSQLIRQST ALTHVFNRFP ENQILVRPPA
ATITTVSENV PALTDHGTLP LRSSISGVQR VTITDARRRT CPYVYKALGI
VAPRVLSSRT F
Rhesus Adenovirus Serotype 51 Penton Base, rhAd51 (A0A0A1EWW1); SEQ ID
NO: 18:
MRRAVRVTPA AYEGPPPSYE SVMGSANVPA TLEAPYVPPR YLGPTEGRNS
IRYSELAPLY DTTKVYLVDN KSADIASLNY QNDHSNFLTT VVQNNDFTPT
EAGTQTINFD ERSRWGGQLK TILHTNMPNI NEFMSTNKFR AKLMVEKSNA
ETRQPRYEWF EFTIPEGNYS ETMTIDLMNN AIVDNYLQVG RQNGVLESDI
GVKFDTRNFR LGWDPVTKLV MPGVYTNEAF HPDIVLLPGC GVDFTQSRLS
NLLGIRKRRP FQEGFQIMYE DLEGGNIPAL LDVSKYEASI QRAKAEGREI
RGDTFAVAPQ DLEIVPLTKD SKDRSYNIIN NTTDTLYRSW FLAYNYGDPE
KGVRSWTILT TTDVTCGSQQ VYWSLPDMMQ DPVTFRPSTQ VSNFPVVGTE
LLPVHAKSFY NEQAVYSQLI RQSTALTHVF NRFPENQILV RPPAPTITTV
SENVPALTDH GTLPLRSSIS GVQRVTITDA RRRTCPYVYK ALGVVAPKVL
SSRTF
rhAd52 (A0A0A1EWX7); SEQ ID NO: 19:
MRRAVRVTPA AYEGPPPSYE SVMGSANVPA TLEAPYVPPR YLGPTEGRNS
IRYSELAPLY DTTKVYLVDN KSADIASLNY QNDHSNFLTT VVQNNDFTPT
EAGTQTINFD ERSRWGGQLK TILHTNMPNI NEFMSTNKFR ARLMVKKVEN
QPPEYEWFEF TIPEGNYSET MTIDLMNNAI VDNYLQVGRQ NGVLESDIGV
KFDTRNFRLG WDPVTKLVMP GVYTNEAFHP DIVLLPGCGV DFTQSRLSNL
LGIRKRRPFQ EGFQIMYEDL EGGNIPALLD VTKYEQSVQR AKAEGREIRG
DTFAVSPQDL VIEPLEHDSK NRSYNLLPNK TDTAYRSWFL AYNYGDPEKG
VRSWTILTTT DVTCGSQQVY WSLPDMMQDP VTFRPSTQVS NFPVVGTELL
PVHAKSFYNE QAVYSQLIRQ STALTHVFNR FPENQILVRP PAPTITTVSE
NVPALTDHGT LPLRSSISGV QRVTITDARR RTCPYVYKAL GVVAPKVLSS
RTF
rhAd53 (A0A0A1EWZ7); SEQ ID NO: NO 20:
MRRAVRVTPA VYAEGPPPSY ESVMGSANVP ATLEAPYVPP RYLGPTEGRN
SIRYSELAPL YDTTKVYLVD NKSADIASLN YQNDHSNFLT TVVQNNDFTP
TEAGTQTINF DERSRWGGQL KTILHTNMPN INEFMSTNKF RARLMVEKTS
GQPPKYEWFE FTIPEGNYSE TMTIDLMNNA IVDNYLQVGR QNGVLESDIG
VKFDTRNFRL GWDPVTKLVM PGVYTNEAFH PDIVLLPGCG VDFTQSRLSN
LLGIRKRRPF QEGFQIMYED LEGGNIPGLL DVPAYEQSLQ QAQEEGRVTR
GDTFATAPNE VVIKPLLKDS KDRSYNIITD TTDTLYRSWF LAYNYGDPEN
GVRSWTILTT TDVTCGSQQV YWSLPDMMQD PVTFRPSTQV SNFPVVGTEL
LPVHAKSFYN EQAVYSQLIR QSTALTHVFN RFPENQILVR PPAPTITTVS
ENVPALTDHG TLPLRSSISG VQRVTITDAR RRTCPYVYKA LGVVAPKVLS
SRTF

The polypeptides of the present invention are not confined to those known specific sequences for amino acid stretches A (minimal ADDobody) or A and B (ADDobody), respectively, forming the alpha-helical domain of the above-referenced adenovirus sub- and serotypes, respectively. Amino acid fragments A and B can also have similar amino acid sequences to the sequences of known adenovirus penton base protomers as long as the sequences of A and B are such that the resulting polypeptide adopts a conformationally stable crown or minimal crown domain under appropriate conditions as further outlined below. Typically, such similar sequences of fragments A and B share an amino acid sequence identity of at least 85%, more preferred at least 90%, even more preferred 95%, particularly preferred at least 98%, most preferred at least 99%, with the respective amino acid sequence of a known adenovirus penton base, preferably those of SEQ ID NOs: 1 to 20, more preferably amino acid stretches A and B as provided in Tables 1 and 2, with the proviso that, in embodiments of the invention where one or more heterologous modifications are present in the RGD region and/or the V loop and/or the floor segment and/or the B loop, said sequence identities as outlined above are to be understood as referring to the adenovirus penton base fragments A and B, preferably of the sequences as outlined above, excluding said RGD loop region and/or said V loop and/or said floor segment and/or said B loop. Also with respect to fragments D, E and F, it is to be understood that these fragments can each have an amino acid sequence similar to the respective parts of the known adenovirus penton base sequences, and preferred sequence identity values given above for fragment A and B also apply to fragments D, E and F.

As used herein, amino acid sequences are stated from N to C terminal using the single letter code of IUPAC, if not otherwise specifically indicated.

A particularly preferred ADDobody of the invention is based on the penton base protein of human adenovirus serotype 3 (hAd3). For preferred sequences as regards the amino acid positions of SEQ ID NO: 1 it is referred to Table 1 (large fragment or fragment A, respectively) and Table 2 (small fragment or fragment B, respectively). The same holds for the minimal ADDobody with respect to the large fragment or fragment A, respectively.

A particularly preferred ADDobody of the invention is based on the penton base protein of chimpanzee adenovirus (ChimpAd). For preferred sequences as regards the amino acid positions of SEQ ID NO: 14 it is referred to Table 1 (large fragment or fragment A, respectively) and Table 2 (small fragment or fragment B, respectively). The same holds for the minimal ADDobody with respect to the large fragment or fragment A, respectively.

As already outlined above, it is one premier embodiment of the invention to include one or more antigens or one or more epitopes thereof, more particularly one or more antigens or one or more epitopes thereof of an infectious agent such as a virus, bacterium or other pathogen, or a tumor or cancer antigen or one or more epitopes thereof, respectfully, into one or more of the sites preferably present in the fragment A and/or B of the ADDobody or minimal ADDobody of the invention as defined above.) As used herein, the term “antigen” or “epitope of an antigen” refers to a structure recognized by molecules of the immune response, e.g. antibodies, T cell receptors (TCRs) etc. In this context, it is also expressis verbis referred to the definitions of “antigen” and “epitope” disclosed in WO 2017/167988 A1 (antigen: page 16, epitope: pages 15 and 16). The heterologous modification(s) present in the polypeptides or engineered adeonovirus base protomers may also be mimotopes of corresponding naturally occurring epitopes.

Antigens or epitopes thereof, respectively, of infectious agents include, but are not limited to, e.g. viral infectious agents, such as HIV, hepatitis viruses such as hepatitis A virus, hepatitis B virus or hepatitis C virus, herpes virus, varicella zoster virus, rubella virus, yellow fever virus, dengue fever virus, flaviviruses (e.g. Zika virus), influenza viruses, Marburg disease virus, Ebola viruses and arboviruses such as Chikungunya virus. Antigens of bacterial infectious agents include, but are not limited to, antigens of e.g. Legionella, Helicobacter, Vibrio, infectious E. coli strains, Staphylococci, Salmonella and Streptococci. Antigens of infectious protozoan pathogens include, but are not limited to, antigens of Plasmodium, Trypanosome, Leishmania and Toxoplasma. Further examples of antigens of pathogenic agents include antigens of fungal pathogens such as antigens of Cryptococcus neoformans, Histoplasma capsulatum, Coccidioides immitis, Blastomyces dermatitidis and Candida albicans.

Specific examples of tumor antigens or epitopes thereof which can be used according to the invention include, but not limited to 707-AP, AFP, ART-4, BAGE, beta-catenin/m, Bcr-abl, CAMEL, CAP-1, CASP-8, CDC27/m, CDK4/m, CEA, CT, Cyp-B, DAM, ELF2M, ETV6-AML1, G250, GAGE, GnT-V, Gp100, HAGE, HER-2/neu, HLA-A*0201-R170I, HPV-E7, HSP70-2M, HAST-2, hTERT (or hTRT), iCE, KIAA0205, LAGE, LDLR/FUT, MAGE, MART-1/Melan-A, MC1R, myosin/m, MUC1, MUM-1, -2, -3, NA88-A, NY-ESO-1, p190 minor bcr-abl, Pml/RAR.alpha., PRAME, PSA, PSM, RAGE, RU1 or RU2, SAGE, SART-1 or SART-3, TEL/AML1, TPI/m, TRP-1, TRP-2, TRP-2/INT2 and VVT1.

A further embodiment of polypeptides of the invention relates to polypeptides wherein at last one of the sites of fragments A and/or B for heterologous modification as defined herein contains antibody sequences or parts of antibodies such as antibody fragments. In this context of the invention the term “antibody” is an immunoglobulin specifically binding to an antigen.

The term “antibody fragment” refers to a part of an antibody which retains the ability of the complete antibody to specifically bind to an antigen. Examples of antibody fragments include, but are not limited to, paratopes, Fab fragments, Fab′ fragments, F(ab′)₂ fragments, heavy chain antibodys, single-domain antibodies (sdAb), scFv fragments, fragment variables (Fv), VH domains, VL domains, nanobodies, IgNARs (immunoglobulin new antigen receptors), di-scFv, bispecific T-cell engagers (BITEs), dual affinity re-targeting (DART) molecules, triple bodies, diabodies, a single-chain diabody and the like.

A “diabody” is a fusion protein or a bivalent antibody which can bind different antigens. A diabody is composed of two single protein chains (typically two scFv fragments) each comprising variable fragments of an antibody. Diabodies therefore comprise two antigen-binding sites and can, thus, target the same (monospecific diabody) or different antigens (bispecific diabody).

The term “single domain antibody” as used in the context of the present invention refers to antibody fragments consisting of a single, monomeric variable domain of an antibody. Simply, they only comprise the monomeric heavy chain variable regions of heavy chain antibodies produced by camelids or cartilaginous fish. Due to their different origins they are also referred to VHH or VNAR (variable new antigen receptor)-fragments. Alternatively, single-domain antibodies can be obtained by monomerization of variable domains of conventional mouse or human antibodies by the use of genetic engineering. They show a molecular mass of approximately 12-15 kDa and thus, are the smallest antibody fragments capable of antigen recognition. Further examples include nanobodies or nanoantibodies.

Antigen-binding entities useful in the context of the invention also include “antibody mimetic” which expression as used herein refers to compounds which specifically bind antigens similar to an antibody, but which compounds are structurally unrelated to antibodies. Usually, antibody mimetics are artificial peptides or proteins with a molar mass of about 3 to 20 kDa which comprise one, two or more exposed domains specifically binding to an antigen. Examples include inter alia the LACI-D1 (lipoprotein-associated coagulation inhibitor); affilins, e.g. human-y B crystalline or human ubiquitin; cystatin; Sac7D from Sulfolobus acidocaldarius; lipocalin and anticalins derived from lipocalins; DARPins (designed ankyrin repeat domains); SH3 domain of Fyn; Kunits domain of protease inhibitors; monobodies, e.g. the 10th type III domain of fibronectin; adnectins: knottins (cysteine knot miniproteins); atrimers; evibodies, e.g. CTLA4-based binders, affibodies, e.g. three-helix bundle from Z-domain of protein A from Staphylococcus aureus; Trans-bodies, e.g. human transferrin; tetranectins, e.g. monomeric or trimeric human C-type lectin domain; microbodies, e.g. trypsin-inhibitor-II, affilins; armadillo repeat proteins. Nucleic acids and small molecules are sometimes considered antibody mimetics as well (aptamers), but not artificial antibodies, antibody fragments and fusion proteins composed from these. Common advantages over antibodies are better solubility, tissue penetration, stability towards heat and enzymes, and comparatively low production costs.

Especially in the context of antigens or epitopes thereof as well as antibodies or antibody fragments, but also with respect to any protein-protein interaction such as receptor-ligand binding, included into the inventive polypeptides in one or more of the sites for heterologous modification in fragment A and/or fragment B of the ADDobody or minimal ADDobody, respectively, but also with respect to any protein-protein interaction such as receptor-ligand binding, it is possible to include a selection and/or evolutionary process for providing target binding-optimized sequences such as optimized antigens or epitopes thereof to exert an improved immune response thereto (cf. FIG. 2) or for providing optimized recognition sequences for antigens or epitopes thereof, especially for generating molecules having multiple binding sites for the antigen or epitope (FIG. 1).

A preferred selection/optimization process in the context of the invention is ribosome display as outlined in detail in Schaffitzel et al. (2001) in: Protein-Protein Interactions, A Molecular Cloning Manual: In vitro selection and evolution of protein-ligand interaction by ribosome display (Golemis E., ed.), pages 535-567, Cold Spring Harbor Laboratory Press, New York. The ribosome display protocol has the advantage of being carried out completely in vitro at all steps of the selection process. Further possible selection processes are also known in the art and include phage display (Smith (1985) Science 228, 1315-1317; Winter et al. (1994) Annu. Rev. Immunol. 12, 433-455), yeast two-hybrid systems (Fields and Song (19899 Nature 340, 245-246; Chien et al. (1983) Proc. Natl. Acad. Sci. U.S.A. 88, 9578-9582), and cell surface display methods (Georgiu et al. (1993) Trends Biotechnol. 11, 6-10; Boder and Wittrup (1997) Nat. Biotechnol. 15, 553-557) as well as mRNA display, yeast display, and/or baculovirus capsid display.

The ribosome display process can basically be used in two ways for optimization of antigens or other amino acid sequences involved in targeting a specific molecule by use of the polypeptides of the invention. Either, the amino acid sequence for selection and/or optimization to bind to a target molecule can be selected first from an initial library of polypeptides sequences that can be as large as 10¹⁴ individual sequences, more typically 10⁹ to 10¹⁰ sequences, optionally employing evolutionary procedures as described in detail in Schaffitzel et al. (2001), supra. After selection of the optimized sequences binding to the target molecule, the nucleotide sequence encoding it/them is/are cloned into an appropriate vector of the invention such that a polypeptide is expressed where the optimized amino acid is included in one or more sites as defined above (RDG region and/or V loop and/or floor region and/or B loop).

According to an alternative embodiment of this aspect of the invention, a library of potential candidate binding sequences is directly cloned into a nucleic acid of the invention such that each sequence encodes a polypeptide containing a candidate binding sequence which included in one or more sites as defined above (RDG region and/or V loop and/or floor region and/or B loop). The inventive polypeptides comprising an initial library of candidate binding sequences are than typically expressed in vitro and selection of optimized binding sequences is carried out according to the ribosome display methodology as outlined in detail in Schaffitzel et al. (2001), supra, or any other suitable selection/evolution methodology for candidate amino acid sequence binding to the targeted molecule known in the art such as phage display, mRNA display, yeast display and/or baculovirus capsid display.

As native penton base proteins do, the engineered adenovirus penton base proteins of the invention assemble into pentameric complexes, 12 of which in turn assemble into virus-like particles (VLPs) in a buffer solution of preferably pH about 5.0 to about 8.0. Preferred examples are buffer conditions at or near physiological conditions such as PBS, pH 7.4, or TBS or TBS-T pH 7.2 to 7.6. Under such conditions, the polypeptides of the invention form VLPs at a temperature of about from about 20 to about 42° C. The present invention is also directed to such pentameric complexes and VLPs.

Further subject matter of the invention is a nucleic acid coding for an ADDobody, minimal ADDobody or engineered adenovirus protein as defined herein.

According to the present invention, the terms “nucleic acid” and “polynucleotide” are used interchangeably and refer to DNA, RNA or species containing one or more nucleotide analogues. Preferred nucleic acids or polynucleotides according to the present invention are DNA, most preferred double-stranded (ds) DNA. Nucleotide sequences of the present disclosure are shown from 5′ to 3′, and the IUPAC single letter code for bases is used, if not otherwise used as indicated.

Embodiments of nucleic acids and vectors, respectively of the invention are also provided for insertion of the versatile heterologous modifications (as, for example, embodied by oligopeptides, polypeptides, proteins etc. as defined herein).

An insertion site in the context of this embodiment of the invention is preferably a recognition sequence of a restriction enzyme or of a homing endonuclease.

Restriction enzyme sites are generally well-known to the skilled person. Preferred examples are as defined above, but restriction sites can be selected from a wide variety and guidance can be found at the various manufacturers of restriction enzymes such as New England Biolabs, Inc., Ipswich, Mass., USA.

Examples of homing endonuclease (HE) sites include, but are not limited to, recognition sequences of PI-SceI, I-CeuI, I-PpoI, I-HmuI I-CreI, I-DmoI, PI-PfuI and I-MsoI, PI-PspI, I-SceI, other LAGLIDAG group members and variants thereof, SegH and Hef or other GIY-YIG homing endonucleases, I-ApeII, I-AniI, Cytochrome b mRNA maturase bl3, PI-Till and PI-TfuII, PI-ThyI and others; see Stoddard B. L. (2005) Q. Rev. Biophys. 38, 49-95. Corresponding enzymes are commercially available, e. g. from New England Biolabs Inc., Ipswich, Mass., USA.

In preferred embodiments of the present invention, the above-defined nucleic acid additionally comprises at least one site for integration of the nucleic acid into a vector or host cell. The integration site may allow for a transient or genomic incorporation.

With respect to the integration into a vector, in particular into a plasmid or virus, the integration site is preferably compatible for integration of the nucleic acid into an adenovirus, adeno-associated virus (AAV), autonomous parvovirus, herpes simplex virus (HSV), retrovirus, rhadinovirus, Epstein-Barr virus, lentivirus, semliki forest virus or baculovirus.

Particularly preferred integration sites that may be incorporated into the nucleic acid of the present invention can be selected from the transposon element of Tn7, λ-integrase specific attachment sites and site-specific recombinases (SSRs), in particular LoxP site or FLP recombinase specific recombination (FRT) site. Further preferred mechanisms for integration of the nucleic acid according to the invention are specific homologous recombination sequences such as lef2-603/Orf1629.

In further preferred embodiments of the present invention, the nucleic acid as described herein additionally contains one or more resistance markers for selecting against otherwise toxic substances. Preferred examples of resistance markers useful in the context of the present invention include, but are not limited to, antibiotics such as ampicillin, chloramphenicol, gentamycin, spectinomycin, and kanamycin resistance markers.

The nucleic acid of the present invention may also contain one or more ribosome binding site(s) (RBS)

Further subject-matter of the present invention relates to a vector comprising a nucleic acid as defined above.

Preferred vectors of the present invention are plasmids, expression vectors, transfer vectors, more preferred eukaryotic gene transfer vectors, transient or viral vector-mediated gene transfer vectors. Other vectors according to the invention are viruses such as adenovirus vectors, adeno-associated virus (AAV) vectors, autonomous parvovirus vectors, herpes simplex virus (HSV) vectors, retrovirus vectors, rhadinovirus vectors, Epstein-Barr virus vectors, lentivirus vectors, semliki forest virus vectors and baculovirus vectors.

Baculovirus vectors suitable for integrating a nucleic acid according to the invention (e.g. present on a suitable plasmid such as a transfer vector) are also subject matter of the present invention and preferably contain site-specific integration sites such as a Tn7 attachment site (which may be embedded in a lacZ gene for blue/white screening of productive integration) and/or a LoxP site. Further preferred baculovirus according to the invention contain (alternative to or in addition to the above-described integration sites) a gene for expressing a substance toxic for host flanked by sequences for homologous recombination. An example for a gene for expressing a toxic substance is the diphtheria toxin A gene. A preferred pair of sequences for homologous recombination is e.g. Isf2-603/Orf1629. The baculovirus can also contain further marker gene(s) as described above, including also fluorescent markers such as GFP, YFP and so on. Specific examples of corresponding baculovirus are, for example disclosed in WO 2010/100278 A1.

Further applicable vectors for use in the invention are disclosed in WO 2005/085456 A1.

Vectors useful in prokaryotic host cells comprise, preferably besides the above-exemplified marker genes (one or more thereof), an origin of replication (ori). Examples are BR322, ColE1, and conditional origins of replication such as OriV and R6Ky, the latter being a preferred conditional origin of replication which makes the propagation of the vector of the present application dependent on the pir gene in a prokaryotic host. OriV makes the propagation of the vector of the present application dependent on the trfA gene in a prokaryotic host.

Furthermore, the present invention is directed to a (recombinant) host cell containing a nucleic acid of the invention and/or a vector of the present invention.

The host cells may be prokaryotic or eukaryotic. Eukaryotic host cells may for example be mammalian cells, preferably human cells. Examples of human host cells include, but are not limited to, HeLa, Huh7, HEK293, HepG2, KATO-III, IMR32,

MT-2, pancreatic β-cells, keratinocytes, bone-marrow fibroblasts, CHP212, primary neural cells, W12, SK-N-MC, Saos-2, WI38, primary hepatocytes, FLC4, 143TK, DLD-1, embryonic lung fibroblasts, primery foreskin fibroblasts, MRCS, and MG63 cells. Further preferred host cells of the present invention are porcine cells, preferably CPK, FS-13, PK-15 cells, bovine cells, preferably MDB, BT cells, bovine cells, such as FLL-YFT cells. Other eukaryotic cells useful in the context of the present invention are C. elegans cells. Further eukaryotic cells include yeast cells such as S. cerevisiae, S. pombe, C. albicans and P. pastoris. Furthermore, the present invention is directed to insect cells as host cells which include cells from S. frugiperda, more preferably Sf9, Sf21, Express Sf+, High Five H5 cells, and cells from D. melanogaster, particularly S2 Schneider cells. Further host cells include Dictyostelium discoideum cells and cells from parasites such as Leishmania spec.

Prokaryotic hosts according to the present invention include bacteria, in particular E. coli such as commercially available strains like TOP10, DH5α, HB101. BL21(DE3) etc.

The person skilled in the art is readily able to select appropriate vector construct/host cell pairs for appropriate propagation and/or transfer of the nucleic acid elements according to the present invention into a suitable host. Specific methods for introducing appropriate vector elements and vectors into appropriate host cells are equally known to the art and methods can be found in the latest edition of Ausubel et al. (ed.) Current Protocols In Molecular Biology, John Wiley & Sons, New York, USA.

In preferred embodiments of the present invention, the vector as defined above additionally comprises a site for site specific recombinases (SSRs), preferably one or more LoxP sites for Cre-lox specific recombination. In further preferred embodiments, the vector according to the present invention comprises a transposon element, preferably a Tn7 attachment site.

It is further preferred that the attachment site as defined above is located within a marker gene. This arrangement makes it feasible to select for successfully integrated sequences into the attachment site by transposition. According to preferred embodiments, such a marker gene is selected from luciferase, β-GAL, CAT, fluorescent encoding protein genes, preferably GFP, BFP, YFP, CFP and their variants, and the lacZa gene.

The present invention also provides the polypeptide, the nucleic acid encoding such a polypeptide, the vector containing a polypeptide-encoding nucleic acid, the host cell comprising such a vector as well as the VLP as defined above for use as a medicament, in particular for use in the treatment and/or prevention of an infectious disease, an immune disease, tumor or cancer.

Therefore, the present invention is also directed to pharmaceutical compositions comprising a polypeptide as defined herein, a nucleic acid encoding such a polypeptide, a vector containing a polypeptide-encoding nucleic, a host cell comprising such a vector or a VLP as described above, optionally together with at least one pharmaceutically acceptable carrier, excipient and/or diluent.

Generally, the preparation of pharmaceutical compositions in the context of the present invention, their dosages and their routes of administration are known to the skilled person, and guidance can be found in the latest edition of Remington's Pharmaceutical Sciences (Mack publishing Co., Eastern, Pa., USA).

The pharmaceutical compositions of the invention contain a therapeutically effective amount of the active ingredient as outlined above. The therapeutically effective amount depends on the active ingredient and in particular on the route of administration. The pharmaceutical composition according to the invention will preferably be applied by parenteral administration, in particular by infusion such as intravenous, intraarterial or intraosseous infusion, or by injection, e.g. intravenous, intraarterial, intraperitoneal, intramuscular, intradermal, subcutaneous or intrathecal injection. In the case of anti-tumor therapy, the pharmaceutical composition such as a pharmaceutical composition containing VLPs according to the invention, can also be administered by intra-tumoral injection.

Inventive solutions for injection or infusion typically contain VLPs of the invention in water or an aqueous buffer solution, preferably an isotonic buffer at physiological pH. Liquid pharmaceutical compositions of the invention may contain further ingredients such as pharmaceutically acceptable stabilizers, suspending aids, emulsifyers and the likes. Further ingredients of the pharmaceutical composition of the invention are adjuvants, in particular in the context of application of the constructs of the invention for vaccination purposes.

Further subject matter of the invention are methods of treatment making use of the beneficial properties of the polypeptides, nucleic acids, host cells, vectors and/or VLPs of the invention. In a preferred embodiment, the invention provides a method for the prevention and/or treatment of an infectious disease comprising the step of administering to a subject, preferably a human, a therapeutically effective amount of the pharmaceutical composition as defined above, wherein the pharmaceutical composition comprises VLPs of the invention containing antigens or epitopes thereof, of the infective agent causing the infectious disease. Another embodiment is a method for preventing and/or treating a tumor or cancer disease the step of administering a therapeutically effective amount of the pharmaceutical composition as defined above to a subject, preferably a human, wherein the pharmaceutical composition comprises VLPs of the invention containing one or more tumor antigens or epitopes thereof.

According to another preferred embodiment, the invention provides a method for the prevention and/or treatment of an infectious disease comprising the step of administering to a subject, preferably a human, a therapeutically effective amount of the pharmaceutical composition as defined above, wherein the pharmaceutical composition comprises VLPs of the invention containing one or more antibodies or antibody fragments such as a paratope thereof, recognizing an antigen, in particular an epitope, of the infective agent causing the infectious disease. Another embodiment is a method for preventing and/or treating a tumor or cancer disease the step of administering a therapeutically effective amount of the pharmaceutical composition as defined above to a subject, preferably a human, wherein the pharmaceutical composition comprises VLPs of the invention one or more antibodies or antibody fragments such as a paratope thereof, recognizing a tumor or cancer antigen, in particular an epitope thereof.

The present invention is further directed to a method for producing the polypeptide as described herein comprising the step of cultivating the recombinant host cell in a suitable medium, wherein the host cell comprises a vector which comprises a nucleic encoding the polypeptide, under conditions allowing the expression of said polypeptide.

Preferably, the method for producing the polypeptide of the invention further comprises the step of recovering the expressed polypeptide from the host cells and/or the medium. Even more preferred, the method also comprises the step of purifying the recovered polypeptide by purification means known in the art such as centrifugation, gel chromatography, ion exchange chromatography, affinity chromatography etc.

The invention also provides a method for producing a VLP as defined herein comprising the step of incubating a solution of the polypeptide under conditions allowing the assembly of the polypeptide into a VLP as outlined before. The proper formation of VLPs can be tested by inspecting a sample solution with an electron microscope.

### BRIEF DESCRIPTION OF THE DRAWINGS

FIG. 1 shows a schematic representation illustrating the use of the adenovirus penton base protein crown domain of the invention (the “ADDobody”) to generate high affinity mono- and/or multivalent binder molecules by selection/evolution from a randomized ADDobody library. An ADDobody library can be generated by randomizing a selection or all of the loops. From this ADDobody library, specific binders can be identified that can bind to any antigen by applying selection/evolution techniques, such as phage display, mRNA display, yeast display, baculovirus capsid display or ribosome display or others.

FIG. 2 shows a schematic representation illustrating the use of the adenovirus penton base protein crown domain of the invention (the “ADDobody”) to generate multivalent vaccines by reverse vaccinology. The crown is shown schematically on the extreme left. The crown domain is colored in green. Four distinct sites have been identified for insertion of heterologous peptide and polypeptide sequences in ADDobody (abbreviated here schematically as ‘loops’). Randomization of these loops yields an ADDobody library that can be utilized to identify loop sequences specifically bound by antibodies prevalent in sera of infected patients or immunized animals. Identification of such epitopic sequences can be used to generate mono- or multivalent ADDOmer superantigens for therapeutic purposes.

FIG. 3 shows (A) Dodecahedra formed by base proteins of certain Adenovirus serotypes represent a versatile scaffold. The high resolution cryo-EM structure (6HCR) is shown. The view is down a central penton axis. Each color stands for a different penton base protein (B) The ADDomer comprises 60 copies of the adenovirus penton base protein. The penton base protein has a two-domain architecture; a crown domain (top) and a multimerization domain (below). The multimerization domain adopts a jellyroll fold. The crown domain comprises exposed highly variable loops. The domains can be separated into two independent protein entities. The crown domain itself can be produced and purified in large quantity. In addition, the crown domain can be engineered to allow display of heterologous peptide sequences (see FIG. 1 and FIG. 2). (C) The crown domain is represented in this schematic view in FIGS. 1 and 2. Removal of the multimerization domain yields an autonomous protein domain according to the invention (the “ADDobody”) which can be produced in large quantity, purified and crystallized.

FIG. 4 shows a schematic representation of the construct pPROEX HTb ADDomer2_Head for expression of an ADDobody of the invention based on human Adenovirus serotype 3 (Adh3).

FIG. 5 shows a MonoQ elution profile of the ADDobody construct ADDomer2_Head

FIG. 6 shows a size exclusion chromatogram of the MonoQ-eluted ADDobody construct ADDomer2_Head

FIG. 7 shows a SDS polyacrylamide gel analysis of the peak fractions of the size exclusion chromatography according to FIG. 6.

FIG. 8 shows a SDS polyacryl amide gel analysis of a sample of the pooled peak fractions of the size exclusion chromatography according to FIG. 6 before and after freeze drying.

FIG. 9 shows an exemplary single crystal of the ADDobody construct ADDomer2_Head.

FIG. 10 shows top views of the X-ray solution structure of the ADDobody construct ADDomer2_Head.

FIG. 11 shows top views of the X-ray solution structure of the ADDobody construct ADDomer2_Head without unit cell.

FIG. 12 shows side views of the X-ray solution structure of the ADDobody construct ADDomer2_Head.

FIG. 13 shows side views of the X-ray solution structure of the ADDobody construct ADDomer2_Head without unit cell.

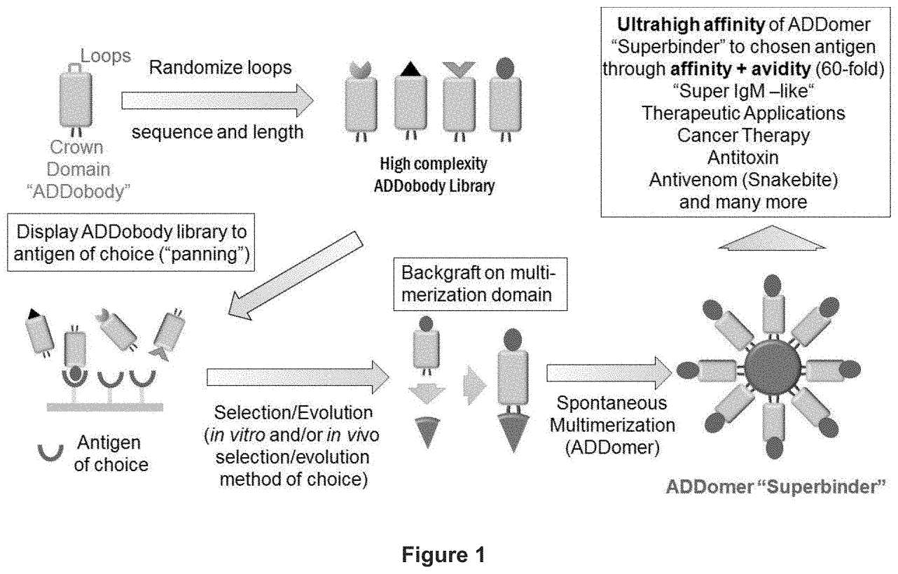

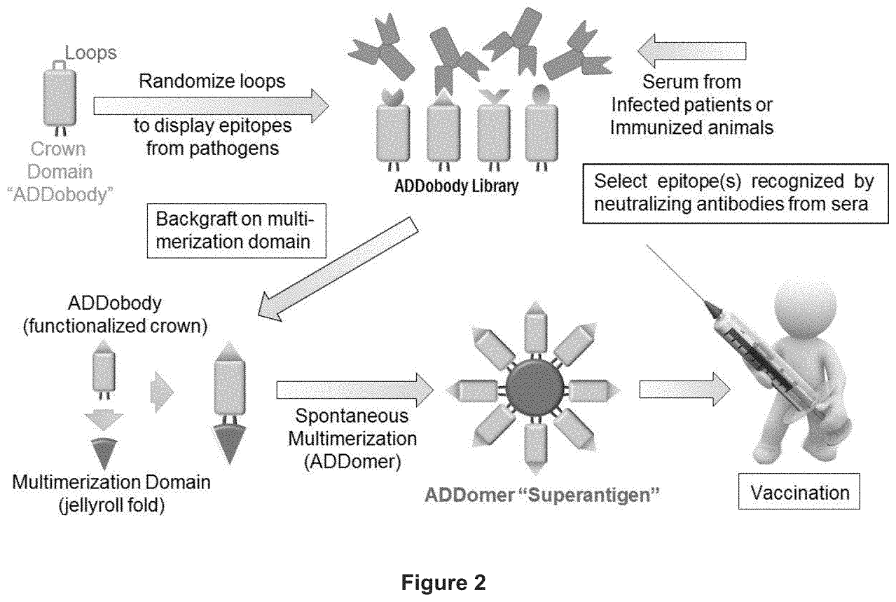

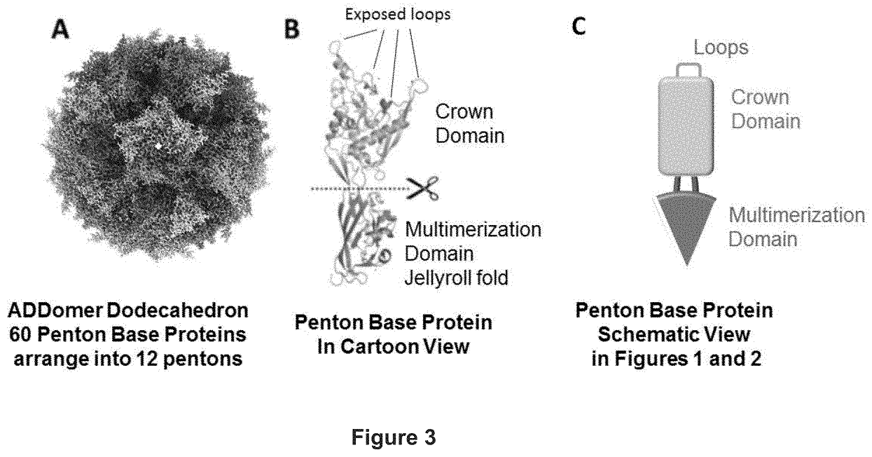

### DETAILED DESCRIPTION OF THE INVENTION

The present invention Is further illustrated by the following non-limiting examples:

### Example 1

Cloning, Expression and Purification of Adenovirus Penton Base Crown Domain of Human Adenovirus Serotype 3 (hAd3)

The nucleotide sequence coding for an ADDobody of the invention (named ADDomer2_Head) equipped with a His-Tag sequence

(SEQ ID NO: 27)
MSYYHHHHHHDYDIPTTENLYFQGAMGSGIQPNVNEYMESNKFKARVMVS
RKAPEGVTVNDTYDHKEDILKYEWFEFILPEGNFSATMTIDLMNNAIIDN
YLEIGRQNGVLESDIGVKFDTRNFRLGWDPETKLIMPGVYTYEAFHPDIV
LLPGCGVDFTESRLSNLLGIRKRHPFQEGFKIMYEDLEGGNIPALLDVTA
YEESKKDTTTETTTKKELKIQPLEKDSKSRSYNVLEDKINTAYRSWYLSY
NYGNPEKGIRSWTLLTTSDVTCGANGDSGNPVFSKSFYNEQAVYSQQLRQ
ATSLTHVFNRFPENQILIRPPAPTITTVSENVP

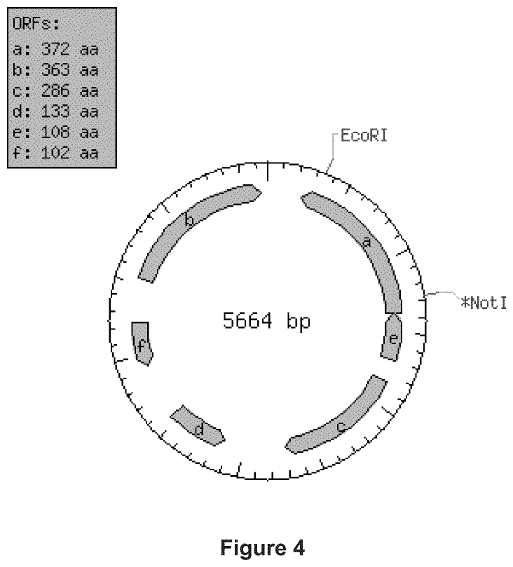

was cloned in expression vector pProEx using the restriction sites shown in FIG. 4.

The resulting vector has the following sequence (ORF start and stop shown in bold, coding sequence in lower characters):

(SEQ ID NO: 31)
GTTTGACAGCTTATCATCGACTGCACGGTGCACCAATGCTTCTGGCGTCAGGCAGCCATCGG
AAGCTGTGGTATGGCTGTGCAGGTCGTAAATCACTGCATAATTCGTGTCGCTCAAGGCGCAC
TCCCGTTCTGGATAATGTTTTTTGCGCCGACATCATAACGGTTCTGGCAAATATTCTGAAAT
GAGCTGTTGACAATTAATCATCCGGTCCGTATAATCTGTGGAATTGTGAGCGGATAACAATT
TCACACAGGAAACAGACCATGTCGTACTACCATCACCATCACCATCACGATTACGATATCCC
AACGACCGAAAACCTGTATTTTCAGGGCGCCATGGGATCCGGAATTCaaccgaacgtgaacg
aatatatgtttagcaacaaatttaaagcgcgcgtgatggtgagccgcaaagcgccggaaggc
gtgaccgtgaacgatacctatgatcataaagaagatattctgaaatatgaatggtttgaatt
tattctgccggaaggcaactttagcgcgaccatgaccattgatctgatgaacaacgcgatta
ttgataactatctggaaattggccgccagaacggcgtgctggaaagcgatattggcgtgaaa
tttgatacccgcaactttcgcctgggctgggatccggaaaccaaactgattatgccgggcgt
gtatacctatgaagcgtttcatccggatattgtgctgctgccgggctgcggcgtggatttta
ccgaaagccgcctgagcaacctgctgggcattcgcaaacgccatccgtttcaggaaggcttt
aaaattatgtatgaagatctggaaggcggcaacattccggcgctgctggatgtgaccgcgta
tgaagaaagcaaaaaagataccaccaccgaaaccaccaccaaaaaagaactgaaaattcagc
cgctggaaaaagatagcaaaagccgcagctataacgtgctggaagataaaattaacaccgcg
tatcgcagctggtatctgagctataactatggcaacccggaaaaaggcattcgcagctggac
cctgctgaccaccagcgatgtgacctgcggcgcgaacggcgatagcggcaacccggtgttta
gcaaaagcttttataacgaacaggcggtgtatagccagcagctgcgccaggcgaccagcctg
acccatgtgtttaaccgctttccggaaaaccagattctgattcgcccgccggcgccgaccat
taccaccgtgagcgaaaacgtgccgTGATAAGCGGCCGCTTTCGAATCTAGAGCCTGCAGTC
TCGAGGCATGCGGTACCAAGCTTGGCTGTTTTGGCGGATGAGAGAAGATTTTCAGCCTGATA
CAGATTAAATCAGAACGCAGAAGCGGTCTGATAAAACAGAATTTGCCTGGCGGCAGTAGCGC
GGTGGTCCCACCTGACCCCATGCCGAACTCAGAAGTGAAACGCCGTAGCGCCGATGGTAGTG
TGGGGTCTCCCCATGCGAGAGTAGGGAACTGCCAGGCATCAAATAAAACGAAAGGCTCAGTC
GAAAGACTGGGCCTTTCGTTTTATCTGTTGTTTGTCGGTGAACGCTCTCCTGAGTAGGACAA
ATCCGCCGGGAGCGGATTTGAACGTTGCGAAGCAACGGCCCGGAGGGTGGCGGGCAGGACGC
CCGCCATAAACTGCCAGGCATCAAATTAAGCAGAAGGCCATCCTGACGGATGGCCTTTTTGC
GTTTCTACAAACTCTTTTTGTTTATTTTTCTAAATACATTCAAATATGTATCCGCTCATGAG
ACAATAACCCTGATAAATGCTTCAATAATATTGAAAAAGGAAGAGTATGAGTATTCAACATT
TCCGTGTCGCCCTTATTCCCTTTTTTGCGGCATTTTGCCTTCCTGTTTTTGCTCACCCAGAA
ACGCTGGTGAAAGTAAAAGATGCTGAAGATCAGTTGGGTGCACGAGTGGGTTACATCGAACT
GGATCTCAACAGCGGTAAGATCCTTGAGAGTTTTCGCCCCGAAGAACGTTTTCCAATGATGA
GCACTTTTAAAGTTCTGCTATGTGGCGCGGTATTATCCCGTGTTGACGCCGGGCAAGAGCAA
CTCGGTCGCCGCATACACTATTCTCAGAATGACTTGGTTGAGTACTCACCAGTCACAGAAAA
GCATCTTACGGATGGCATGACAGTAAGAGAATTATGCAGTGCTGCCATAACCATGAGTGATA
ACACTGCGGCCAACTTACTTCTGACAACGATCGGAGGACCGAAGGAGCTAACCGCTTTTTTG
CACAACATGGGGGATCATGTAACTCGCCTTGATCGTTGGGAACCGGAGCTGAATGAAGCCAT
ACCAAACGACGAGCGTGACACCACGATGCCTACAGCAATGGCAACAACGTTGCGCAAACTAT
TAACTGGCGAACTACTTACTCTAGCTTCCCGGCAACAATTAATAGACTGGATGGAGGCGGAT
AAAGTTGCAGGACCACTTCTGCGCTCGGCCCTTCCGGCTGGCTGGTTTATTGCTGATAAATC
TGGAGCCGGTGAGCGTGGGTCTCGCGGTATCATTGCAGCACTGGGGCCAGATGGTAAGCCCT
CCCGTATCGTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTTACTCATA
TATACTTTAGATTGATTTAAAACTTCATTTTTAATTTAAAAGGATCTAGGTGAAGATCCTTT
TTGATAATCTCATGACCAAAATCCCTTAACGTGAGTTTTCGTTCCACTGAGCGTCAGACCCC
GTAGAAAAGATCAAAGGATCTTCTTGAGATCCTTTTTTTCTGCGCGTAATCTGCTGCTTGCA
AACAAAAAAACCACCGCTACCAGCGGTGGTTTGTTTGCCGGATCAAGAGCTACCAACTCTTT
TTCCGAAGGTAACTGGCTTCAGCAGAGCGCAGATACCAAATACTGTCCTTCTAGTGTAGCCG
TAGTTAGGCCACCACTTCAAGAACTCTGTAGCACCGCCTACATACCTCGCTCTGCTAATCCT
GTTACCAGTGGCTGCTGCCAGTGGCGATAAGTCGTGTCTTACCGGGTTGGACTCAAGACGAT
AGTTACCGGATAAGGCGCAGCGGTCGGGCTGAACGGGGGGTTCGTGCACACAGCCCAGCTTG
GAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGCA
CGAGGGAGCTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAAAAACGCCAG
CAACGCGGCCTTTTTACGGTTCCTGGCCTTTTGCTGGCCTTTTGCTCACATGTTCTTTCCTG
CGTTATCCCCTGATTCTGTGGATAACCGTATTACCGCCTTTGAGTGAGCTGATACCGCTCGC
CGCAGCCGAACGACCGAGCGCAGCGAGTCAGTGAGCGAGGAAGCGGAAGAGCGCCTGATGCG
GTATTTTCTCCTTACGCATCTGTGCGGTATTTCACACCGCATAATTTTGTTAAAATTCGCGT
TAAATTTTTGTTAAATCAGCTCATTTTTTAACCAATAGGCCGAAATCGGCAAAATCCCTTAT
AAATCAAAAGAATAGACCGAGATAGGGTTGAGTGTTGTTCCAGTTTGGAACAAGAGTCCACT
ATTAAAGAACGTGGACTCCAACGTCAAAGGGCGAAAAACCGTCTATCAGGGCGATGGCCCAC
TACGTGAACCATCACCCTAATCAAGTTTTTTGGGGTCGAGGTGCCGTAAAGCACTAAATCGG
AACCCTAAAGGGAGCCCCCGATTTAGAGCTTGACGGGGAAAGCCGGCGAACGTGGCGAGAAA
GGAAGGGAAGAAAGCGAAAGGAGCGGGCGCTAGGGCGCTGGCAAGTGTAGCGGTCACGCTGC
GCGTAACCACCACACCCGCCGCGCTTAATGCGCCGCTACAGGGCGCGTCCCATTCGCCATTC
AGGCTGCTATGGTGCACTCTCAGTACAATCTGCTCTGATGCCGCATAGTTAAGCCAGTACCA
GTCACGTAGCGATATCGGAGTGTATACACTCCGCTATCGCTACGTGACTGGGTCATGGCTGC
GCCCCGACACCCGCCAACACCCGCTGACGCGCCCTGACGGGCTTGTCTGCTCCCGGCATCCG
CTTACAGACAAGCTGTGACCGTCTCCGGGAGCTGCATGTGTCAGAGGTTTTCACCGTCATCA
CCGAAACGCGCGAGGCAGCAGATCAATTCGCGCGCGAAGGCGAAGCGGCATGCATTTACGTT
GACACCATCGAATGGTGCAAAACCTTTCGCGGTATGGCATGATAGCGCCCGGAAGAGAGTCA
ATTCAGGGTGGTGAATGTGAAACCAGTAACGTTATACGATGTCGCAGAGTATGCCGGTGTCT
CTTATCAGACCGTTTCCCGCGTGGTGAACCAGGCCAGCCACGTTTCTGCGAAAACGCGGGAA
AAAGTGGAAGCGGCGATGGCGGAGCTGAATTACATTCCCAACCGCGTGGCACAACAACTGGC
GGGCAAACAGTCGTTGCTGATTGGCGTTGCCACCTCCAGTCTGGCCCTGCACGCGCCGTCGC
AAATTGTCGCGGCGATTAAATCTCGCGCCGATCAACTGGGTGCCAGCGTGGTGGTGTCGATG
GTAGAACGAAGCGGCGTCGAAGCCTGTAAAGCGGCGGTGCACAATCTTCTCGCGCAACGCGT
CAGTGGGCTGATCATTAACTATCCGCTGGATGACCAGGATGCCATTGCTGTGGAAGCTGCCT
GCACTAATGTTCCGGCGTTATTTCTTGATGTCTCTGACCAGACACCCATCAACAGTATTATT
TTCTCCCATGAAGACGGTACGCGACTGGGCGTGGAGCATCTGGTCGCATTGGGTCACCAGCA
AATCGCGCTGTTAGCGGGCCCATTAAGTTCTGTCTCGGCGCGTCTGCGTCTGGCTGGCTGGC
ATAAATATCTCACTCGCAATCAAATTCAGCCGATAGCGGAACGGGAAGGCGACTGGAGTGCC
ATGTCCGGTTTTCAACAAACCATGCAAATGCTGAATGAGGGCATCGTTCCCACTGCGATGCT
GGTTGCCAACGATCAGATGGCGCTGGGCGCAATGCGCGCCATTACCGAGTCCGGGCTGCGCG
TTGGTGCGGATATCTCGGTAGTGGGATACGACGATACCGAAGACAGCTCATGTTATATCCCG
CCGTTAACCACCATCAAACAGGATTTTCGCCTGCTGGGGCAAACCAGCGTGGACCGCTTGCT
GCAACTCTCTCAGGGCCAGGCGGTGAAGGGCAATCAGCTGTTGCCCGTCTCACTGGTGAAAA
GAAAAACCACCCTGGCACCCAATACGCAAACCGCCTCTCCCCGCGCGTTGGCCGATTCATTA
ATGCAGCTGGCACGACAGGTTTCCCGACTGGAAAGCGGGCAGTGAGCGCAACGCAATTAATG
TGAGTTAGCGCGAATTGATCTG

The polypeptide was produced and purified as follows:

### Buffers and Stock Solutions

1000× Ampicillin stock: 1 g Ampicillin dissolved in 10 ml dH₂O, filtered through a 0.2 μm filter
1 M IPTG stock: 2.4 g IPTG is dissolved in 10 ml dH₂O and filtered through a 0.2 μm filter.
10×PBS buffer stock:
   80 g NaCl
   2 g KCl
   7.62 g Na₂HPO₄
   0.77 g KH₂PO₄ in 1 L ultrapure H₂O, set pH 7.4
10×PBS is diluted 1:10 before use to get 1×PBS.

[In the following text, the expression AD3Head is used for ADDobody]

The expression and purification protocol is described per 1 L culture. It should be upscaled accordingly if more expression cultures are required. Normally, 3-5 L expression culture for one big prep should be made, and the purified protein stored frozen.

### 1. BL21(DE3) Bacteria Transformation

pProEX_HTB_AD3Head plasmid encoding for the ADDomer head or ‘crown’ domain (the ADDobody) is transformed into BL21(DE3) competent cells and plated onto LB agarose plate containing ampicillin as selection marker

### 1.1. Plate Preparation:

LB agarose is warmed in microwave oven
Bottle is chilled under running tap water
1000× Ampicillin stock is added (100 μl to 100 ml LB agarose)

### 1.2 Transformation

BL21(DE3) cells are put on ice for 20 min to thaw
5 μl of plasmid DNA (˜0.67 μg) is added to the cells and incubated on ice for 1 h
Heat shock is performed for 45 s at 42° C. and tube is chilled on ice for 10 min
500 μl sterile LB is added to the tube
Cells are incubated for 1 h at 37° C. in incubator rotating at 180 rpm
100 μl of cell culture is plated onto LB agarose plate containing ampicillin and incubated at 37° C. overnight
The remaining 400 μl of cells are incubated overnight at 37° C. in incubator rotating at 180 rpm and kept as backup if re-plating is needed

### 2. Bacterial Preculture

Single colony is inoculated to sterile flask containing 50 ml dYT media with 50 μl ampicillin
Incubated at 37° C. overnight rotating at 180 rpm

### 3. Induction of Expression Cultures

50 ml preculture at OD 3-4 is inoculated to 1 L prewarmed dYT medium containing 1 ml ampicillin (1000× stock)
Cultures are incubated at 37° C. in incubator rotating at 180 rpm
OD measurement is taken hourly until it reaches 1 OD again (takes about 3 h)
Preinduction sample is taken from each shaker flask (see below)
Then culture is induced with 1 ml IPTG stock and incubated at 30° C. for 3 h.
Postinduction sample is taken from each shaker flask (see box below) Run SDS-PAGE gel (12%)

### 4. Cell Harvesting

1 L culture is distributed to two plastic bottles and precisely balanced for centrifugation
Centrifuge at 3500 G for 20 min at 4° C.
Supernatant is discarded and pellet is resuspended in 20 ml used media, then transferred to 50 ml falcon tubes
Centrifuge at 3000 G for 10 min in table-top centrifuge
Media is discarded and Falcon tubes tapped in paper towel to remove rest of media
Pellet is flash-frozen in liquid N₂, stored at −20° C. until purification

### 5. Batch Purification by IMAC Using TALON Resin

Buffers:
   PBS (1×) (see above)
   High salt wash buffer: pH 7.4 (set on ice at 4° C.)
      50 mM TRIS->0.622 g Trizma base
      1 M KCl->7.45 g KCl
      5 mM imidazole->0.034 g imidazole in final volume
      100 ml dH₂O
   Elution buffer: pH 7.4 (set on ice at 4° C.)
      200 mM imidazole->1.36 g in final volume 100 ml
      1×PBS
      Measure harvested pellet weight, add 10 ml 1×PBS per gram pellet and resuspend.
      Sonicate cells two times for 5 min: pulse sonication 10 s on, 15 s off (on ice)
      Centrifuge at 3000 G for 10 min
      Transfer supernatant to new falcons and centrifuge again at 3000 G for 10 min
      Cleared supernatant is loaded on equilibrated TALON resin (see below).

### 5.2. Resin Equilibration (HisPur TALON Resin)

1 ml ‘slurry’ is added to a 15 ml falcon and washed with 5 ml dH₂O, centrifuged at 1000 rpm for 5 min, supernatant is discarded
The slurry is washed with 5 ml 1×PBS, centrifuged at 1000 rpm for 5 min, supernatant is discarded

### 5.3. Binding and Washing Steps

Cleared lysate is loaded on top of the equilibrated resin and incubated overnight at 4° C. in the cold room with constant rotation.
Next day, centrifuge at 1000 rpm for 5 min and collect flow through (FT)
Add High Salt Wash Buffer (7 ml) to the resin, incubate for 20 min at 4° C. rotating in the cold room.
Falcon is centrifuged at 1000 rpm for 5 min, supernatant is collected (HS).
Washing: 7 ml 1×PBS is added to the resin, then centrifuged at 1000 rpm for 5 min, supernatant is collected (W1).
Washing step with 1×PBS is repeated two more times and supernatant is collected (W2, W3).
Resin is resuspended in 1 mL 1×PBS and transferred to a 1.5 ml Eppendorf tube. Centrifuged at 1000 rpm for 5 min, and supernatant is discarded.

### 5.4 Elution

5 ml Elution buffer is added to the resin, incubated for 20 min at 4° C. in the cold room while rotating.
Tube is centrifuged at 13.000 rpm for 5 min, supernatant is collected to fresh tube (E1).
5 ml Elution buffer is added to the resin, incubated for 20 min at 4° C. in the cold room while rotating.
Tube is centrifuged at 13000 rpm for 5 min, supernatant is collected to fresh tube (E2).
5 ml Elution buffer is added to the resin, incubated for 20 min at 4° C. in the cold room while rotating.
Tube is centrifuged at 13000 rpm for 5 min, supernatant is collected to fresh tube (E3).
Resin is resuspended in 1 ml elution buffer (‘Resin’). 12 ul taken.
Samples (12 μl+4 μl PGLB) from each step are run on SDS-PAGE gel (12%).

### 6. Dialysis and TEV Cleavage

Protein concentration is measured with Nanodrop (A280) prior to dialysis and TEV cleavage (use Elution Buffer as blank to correct for imidazol).
Take 12 ul sample (Before diaysis' sample).
20 μl TEV protease is added
Dialyse in dialysis bag in the cold room against 1×PBS with 2 mM □-MeSH (□-MeSH must be handled under the fume hood).
Change to new beaker with fresh 1×PBS w. □-MeSH after 30 min.
Continue dialysis overnight at 4° C.
Take 12 ul sample (After dialysis' sample).
Recover sample comprising ADDobody, measure concentration of protein against dialysis buffer as blank, determine volume of sample and insert in Table I (see Annex).

### 7. Removing Uncut Protein and TEV (Reverse TALON)

Dialysed sample is loaded to 1 ml equilibrated TALON resin (equilibrate resin as described previously) and incubated for 1 h at 4° C. rotating in the cold room.
Concentration is measured with Nanodrop. 12 ul sample taken (‘Before conc’).
Sample is concentrated using am Amicon Millipore Protein concentrator

(MWCO 10 kDa). Centrifugation is performed at 3000 G at 4° C. to 500 ul.
Take 12u1 sample (‘After conc’).
Measure concentration with Nanodrop. Determine volume of sample and insert into Table I (Annex).
Samples (12 μl+4 μl PGLB) from each step are run on SDS-PAGE gel (12%).

### 8. Reverse Ion Exchange Chromatography (IEX)

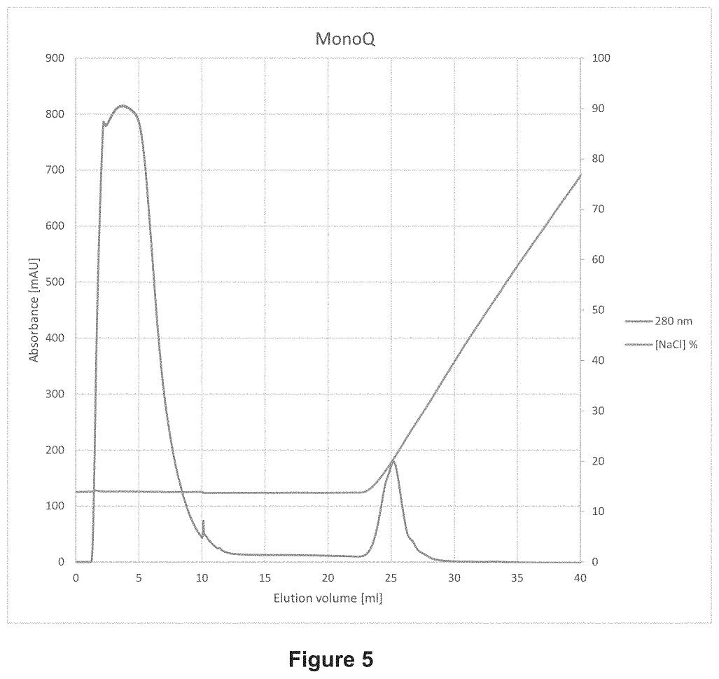

Ion exchange chromatography is performed with AKTA Pure system using Mono Q 5/50 GL column. [Remark: This is a ‘reverse’ IEX as ADDobody drops through the column (FT), impurities in contrast, are expected to bind.]
   Buffer A: 1×PBS (freshly made and filtered)
   Buffer B: 1×PBS with 1 M NaCl
      (for 1 L Buffer B weigh out 58.44 g (1 mol) NaCl and add to 1 L 1×PBS dissolve by stirring).
   Delta column pressure: 4.00 MPa
   Column is equilibrated prior to injection in 1×PBS (15 ml, i.e. 15 column volumes)
   Flow rate 1 ml/min
   Samples are collected with fractionator, fraction volume is 1 ml.
   Column is washed with Buffer A at 1 ml/min flow-rate
   500 μl sample is centrifuged at 13.000 rpm in table-top centrifuge 12 ul probe taken (IEX IN)
   Sample injected.
   Linear gradient applied:
      0-100% B in 20 min
      100% B for 5 min
      0% B for 5 min
      (0% B for 5 min is for reequilibrating the column).
   Collect in 1 ml fractions.
ADDobody elutes in FT 1-10 ml elution volume (and a minor peak around 20 ml elution volume.)
   An exemplary elution profile is shown in FIG. 5.
Combine fractions which comprise ADDobody. Measure concentration with Nanodrop against Buffer A as blank. Determine volume of sample and insert into Table I (Annex).

### 9. Size Exclusion Chromatography (SEC)

Buffer A: 1×PBS (freshly made and filtered)
Fractions from ion exchange runs corresponding to the main peak are pooled and concentrated with Amicon Millipore protein concentrator (MWCO 10 kDa) as described previously.
Important: Be careful not to overconcentrate, the protein might precipitate. If you use 3-5 L expression culture, you might need to use two concentrators, monitor concentration in steps and make sure you do not see precipitation.
Size exclusion chromatography is performed of 500 ul aliquots of concentrated sample with AKTA Pure system using HiLoad 16/600 Superdex 200 pg column.
   Delta column pressure: 0.3 MPa
   Column is equilibrated prior to injection with 1×PBS to total volume 5 ml
   Flow rate: 1 ml/min
   Sample application: loop is emptied with 2 ml
   Sample are collected with fractionation, fraction volume: 1 ml
   500 μl sample aliquots are centrifuged at 13.000 rpm in table-top centrifuge.
   Take 12 ul probe of each injection (SEC IN).
   Inject the rest of the 500 ul.

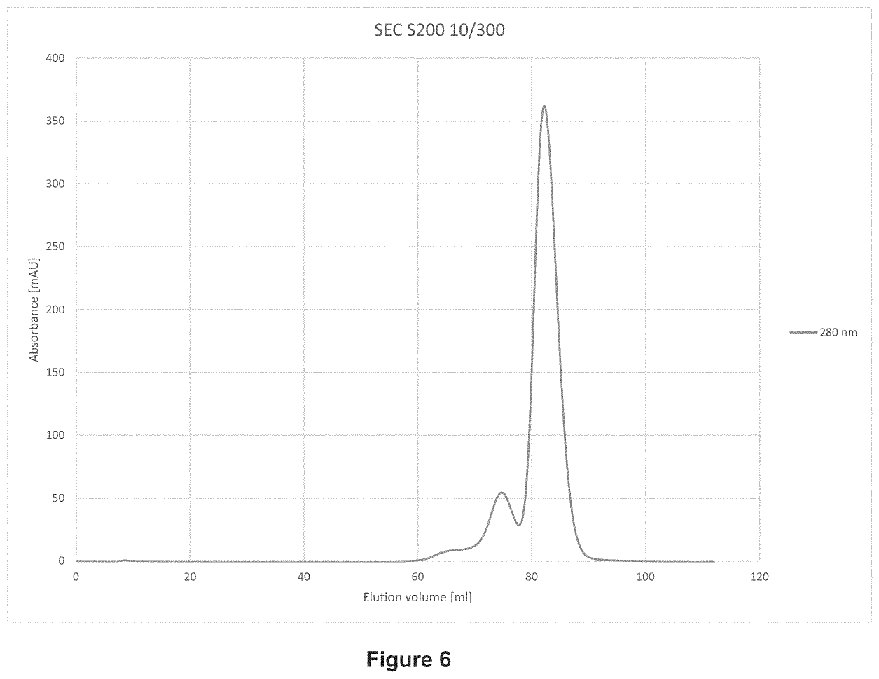

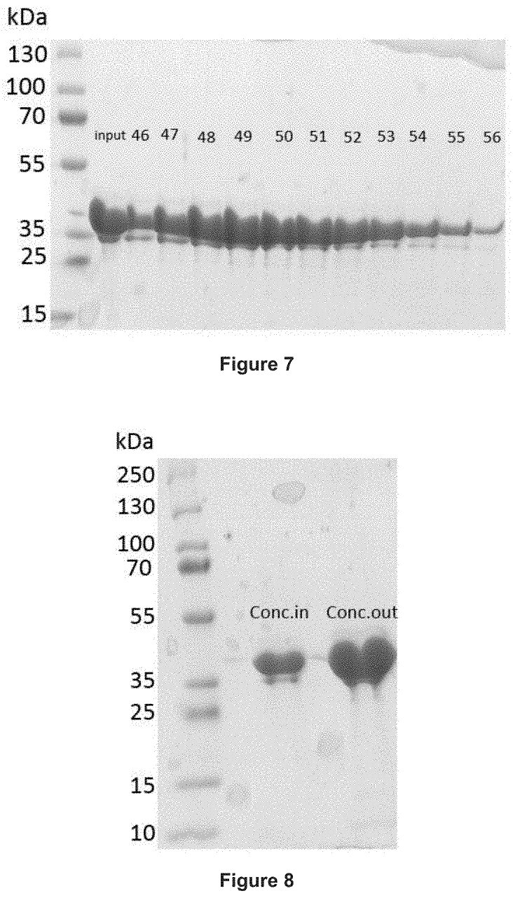

An exemplary chromatogram is shown in FIG. 6 and the SDS polyacryl amide gel analysis of the peak fractions are shown in FIG. 7.

### 10. Freezing and Storage

Fractions from SEC corresponding to the major peak are pooled.
Measure concentration with Nanodrop against Buffer A as blank. Determine volume of sample and insert into Table I (Annex).
Take 12 ul sample (‘ConcFreeze IN’)
Concentrate to 5 mg/ml final concentration using Amicon Millipore protein concentrator (MWCO 10 kDa).
Take 12 ul sample (‘ConcFreeze OUT’)

Protein is flash-frozen in 100 μl aliquots in low protein binding tubes and stored at −20° C.

A SDS polyacrylamide gel analysis of the pooled fractions before and after freezing is shown in FIG. 8 (before freezing: Conc. In; after freezing: Conc. out) shows that the ADDobody construct ADDomer2_Head is stable.

### Example 2

Crystallization and X-Ray Structure Determination of ADDobody Construct ADDomer2_Head

The truncated (i.e. maturated) polypeptide ADDomer2_Head (the amino acid sequence according to SEQ ID NO: 27 lacking the His tag-containing sequence

(SEQ ID NO: 39)
MSYYHHHHHHDYDIPTTENLYF)
(SEQ ID NO: 28)
GAMGSGIQPNVNEYMFSNKFKARVMVSRKAPEGVTVNDTYDHKEDILKYE
WFEFILPEGNFSATMTIDLMNNAIIDNYLEIGRQNGVLESDIGVKFDTRN
FRLGWDPETKLIMPGVYTYEAFHPDIVLLPGCGVDFTESRLSNLLGIRKR
HPFQEGFKIMYEDLEGGNIPALLDVTAYEESKKDTTTETTTKKELKIQPL
EKDSKSRSYNVLEDKINTAYRSWYLSYNYGNPEKGIRSWTLLTTSDVTCG
ANGDSGNPVFSKSFYNEQAVYSQQLRQATSLTHVFNRFPENQILIRPPAP
TITTVSENVP

was crystalized and subjected to X-ray diffraction.

Crystallization conditions: sitting drop, PEG3350 20% (w/v), 0.1 M citrate pH 5.5, protein concentration: 5 mg/ml, drop size: 0.5 ul protein and 0.5 ul reservoir.

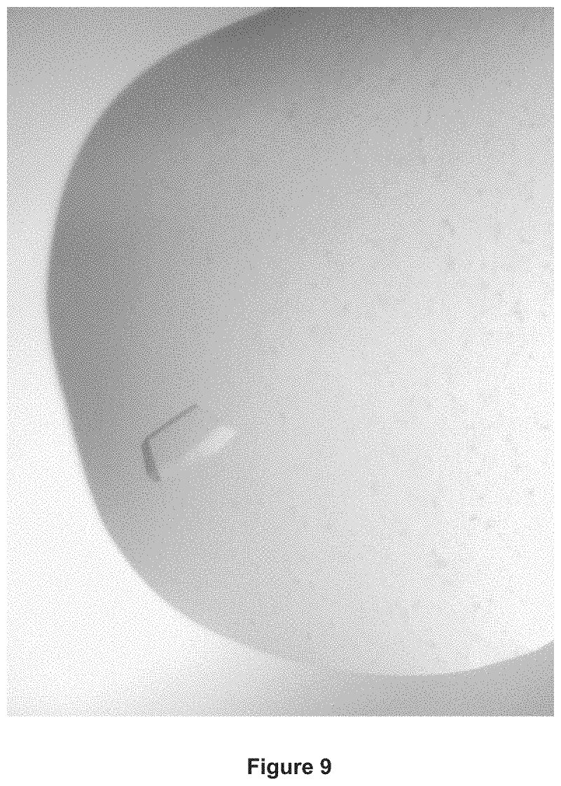

A representative single crystal is shown in FIG. 9. The best crystals diffracted to approximately 4.5 Å.

The X-ray diffraction gave the following results:

Spacegroup P1

a=102.124 b=103.837 c=177.578 α=92.308 β=94.314 γ=112.047

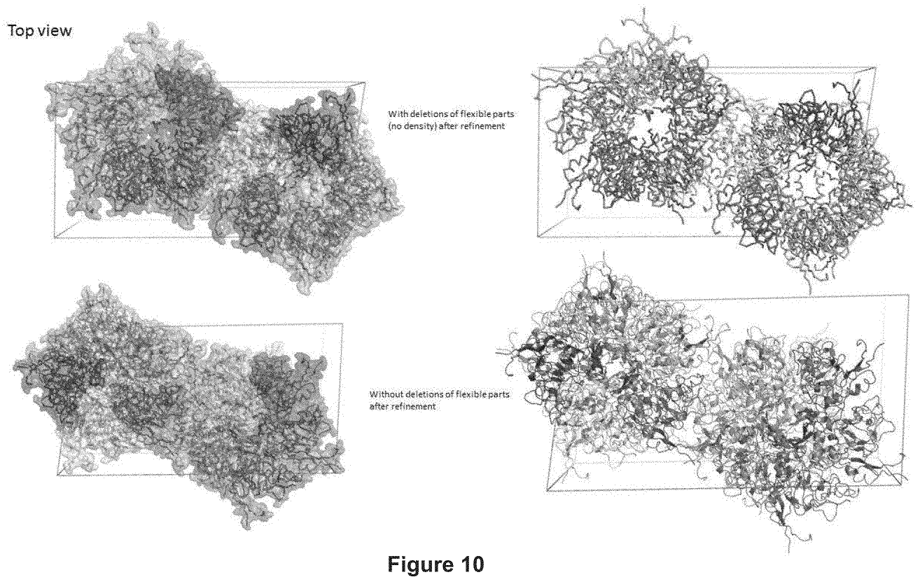

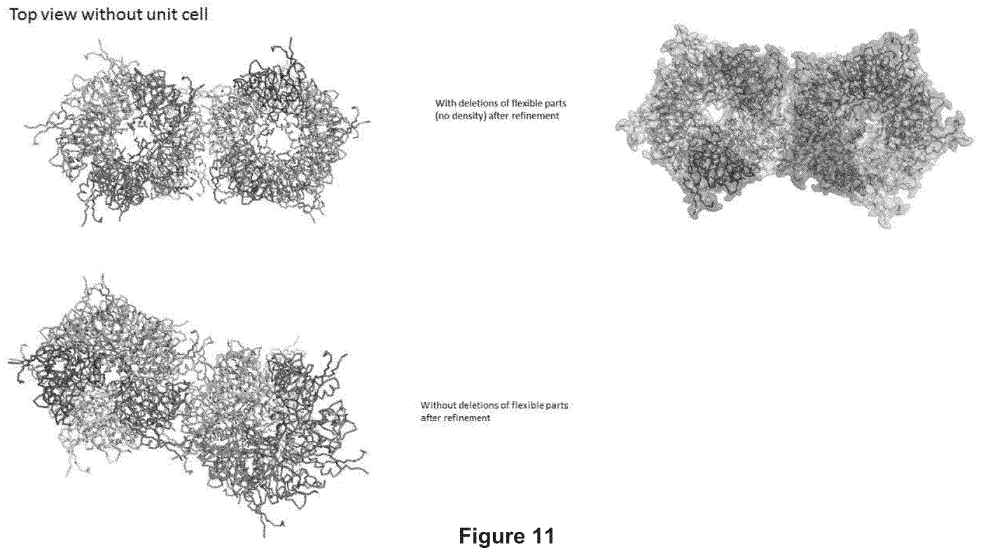

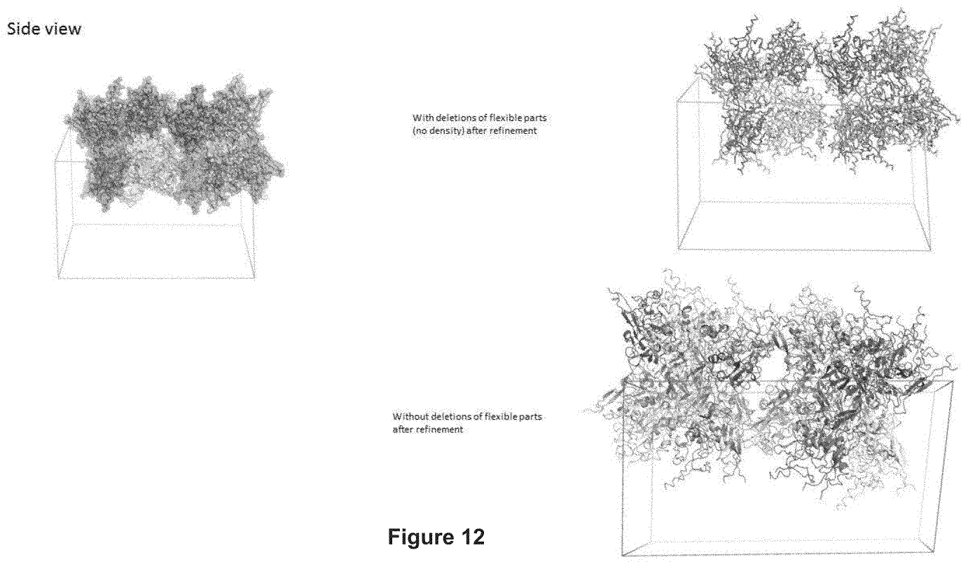

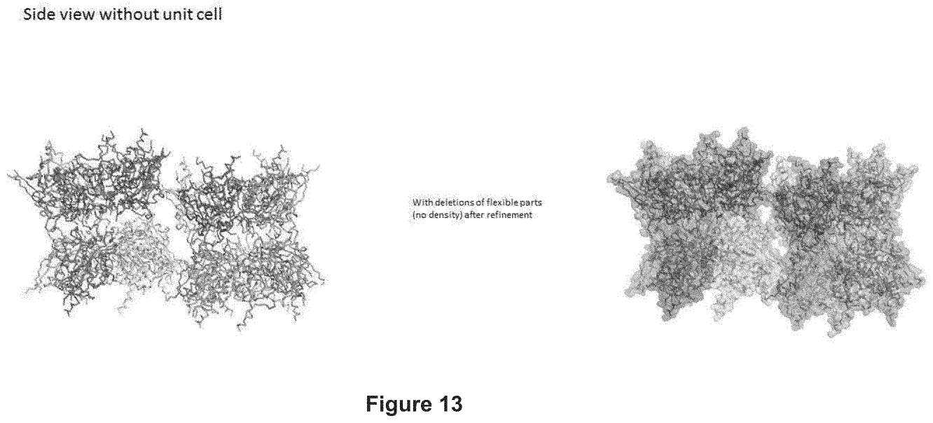

The solution structure is shown in FIGS. 10 to 13.

The unit cell contains 20 ADDobodies present in 2 decamers.

Importantly, the floor region of fragment A and the B loop of fragment B of the ADDobody forms a flexible structure in the isolated ADDobody

### Example 3

Cloning, Expression and Purification of Adenovirus Penton Base Crown Domain of Chimpanzee Adenovirus

The nucleotide sequence coding for a further ADDobody of the invention (named ChimpCrown) equipped with a His-Tag sequence

(SEQ ID NO: 28)
MSYYHHHHHHDYDIPTTENLYFQGTIMHTNMPNVNEFMYSNKFKARVMVS
RKAPEGVTVNDTYDHKEDILEYEWVEFELPEGNFSVTMTIDLMNNAIIDN
YLAVGRQNGVLESDIGVKFDTRNFRLGWDPVTELVMPGVYTNEAFHPDIV
LLPGCGVDFTESRLSNLLGIRKRQPFQEGFQIMYEDLEGGNIPALLDVDA
YEKSKKDTTTETTTKKELKIQPVEKDSKDRSYNVLPDKINTAYRSWYLAY
NYGDPEKGVRSWTLLTTSDVTCGVEQAELLPVYSKSFFNEQAVYSQQLRA
FTSLTHVFNRFPENQILVRPPAPTITTVSENVP

was cloned in expression vector pProEx as outlined in Example 1

Expression and purification was carried out according to the protocol of Example 1.

### Example 4

Cloning, Expression and Purification of Adenovirus Penton Base Crown Domain of Human Adenovirus Serotype 3 (hAd3) Containing a Heterologous Insertion

The nucleotide sequence coding for a modified ADDobody of the invention containing the major neutralizing epitope from Chikungunya virus STKDNFNVYKATRPYLAH (SEQ ID NO: 40) in the B loop of fragment B and replacing the sequence HVFNRF (SEQ ID NO: 41) in the wild-type fragment B of hAd3 penton base (equipped with a His-Tag sequence)

(SEQ ID NO: 42)
MSYYHHHHHHDYDIPTTENLYFQGAMGSGIQPNVNEYMFSNKFKARVMVS
RKAPEGVTVNDTYDHKEDILKYEWFEFILPEGNFSATMTIDLMNNAIIDN
YLEIGRQNGVLESDIGVKFDTRNFRLGWDPETKLIMPGVYTYEAFHPDIV
LLPGCGVDFTESRLSNLLGIRKRHPFQEGFKIMYEDLEGGNIPALLDVTA
YEESKKDTTTETTTKKELKIQPLEKDSKSRSYNVLEDKINTAYRSWYLSY
NYGNPEKGIRSWTLLTTSDVTCGANGDSGNPVFSKSFYNEQAVYSQQLRQ
ATSLTSTKDNFNVYKATRPYLAHPENQILIRPPAPTITTVSENVP

was cloned in expression vector pProEx as outlined in Example 1 Expression and purification was carried out according to the protocol of Example 1.

In summary, the present invention is particularly directed to the following items:
1. An isolated engineered polypeptide comprising the amino acid stretches essentially corresponding to a first and a second fragment of the penton base wherein the first fragment of the polypeptide is present between the first and second amino acid stretches forming the jellyroll fold domain in the full length penton base and wherein the second fragment of the polypeptide is present between the second and third fragments forming the jellyroll fold domain in the full length penton base, respectively, wherein the isolated engineered domain lacks the amino acid stretches forming the jellyroll fold domain of the adenovirus penton base, wherein optionally the first and/or second fragments of the polypeptide contain(s) one or more heterologous modification(s).
2. The polypeptide of item 1 having the structure of the following general formula (I):

N-A-L-B-C (I)
wherein
A represents an amino acid stretch corresponding to the N-terminal amino acid stretch of the adenovirus penton base present between the first and the second amino acid stretch forming the jellyroll fold domain of the adenovirus penton base;
B represents an amino acid stretch corresponding to the C-terminal amino acid stretch of the adenovirus penton base inserted between the second and the third amino acid stretch forming the jellyroll fold domain of the adenovirus penton base;
L represents a chemical group selected from the group consisting of an amino acid, an oligopeptide and a polypeptide;
N may or may not be present, and, if present, represents a chemical group consisting of an amino acid, an oligopeptide and a polypeptide;
C: may or may not be present, and, if present, represents a chemical group consisting of an amino acid, an oligopeptide and a polypeptide;
wherein, optionally, fragment A and/or B contain(s) one or more heterologous modifications.
3. The polypeptide according to any one of items 2 wherein the fragment A comprises an amino acid sequence selected from the group consisting of the amino acid sequences according to the following table and amino acid sequences having an identity of at least 85%, more preferred at least 90%, even more preferred 95%, particularly preferred at least 98%, most preferred at least 99%, with the respective amino acid sequence shown in the following table:

| Sequence | Sequence | | N-terminal | C-terminal |
|---|---|---|---|---|
| based on | according to | | amino acid | amino acid |
| penton base | UniProt | SEQ ID | selected from | selected from |
| protomer of | Acc. No. | NO: | positions | positions |
| hAd3 | Q2Y0H9 | 1 | 130 to 137 | 399 to 405 |
| hAd2 | P03276 | 2 | 130 to 137 | 426 to 432 |
| hAd4 | Q2KSF3 | 3 | 126 to 133 | 380 to 386 |
| hAd5 | P12538 | 4 | 130 to 137 | 426 to 432 |
| hAd7 | Q9JFT6 | 5 | 130 to 137 | 399 to 405 |
| hAd11 | D2DM93 | 6 | 130 to 137 | 416 to 422 |
| hAd12 | P36716 | 7 | 120 to 127 | 352 to 358 |
| hAd17 | F1DT65 | 8 | 117 to 124 | 371 to 377 |
| hAd25 | M0QUK0 | 9 | 125 to 133 | 389 to 395 |
| hAd35 | Q7T941 | 10 | 131 to 138 | 446 to 452 |
| hAd37 | Q912J1 | 11 | 117 to 124 | 373 to 379 |
| hAd41 | F8WQN4 | 12 | 128 to 135 | 362 to 368 |
| gorAd | E5L3Q9 | 13 | 131 to 138 | 417 to 423 |
| ChimpAd | G9G849 | 14 | 126 to 133 | 373 to 379 |
| sAd18 | H8PFZ9 | 15 | 128 to 135 | 354 to 360 |
| sAd20 | F6KSU4 | 16 | 127 to 134 | 359 to 365 |
| sAd49 | F2WTK5 | 17 | 128 to 135 | 357 to 363 |
| rhAd51 | A0A0A1EWW1 | 18 | 125 to 132 | 353 to 359 |
| rhAd52 | A0A0A1EWX7 | 19 | 125 to 132 | 351 to 357 |
| rhAd53 | A0A0A1EWZ7 | 20 | 126 to 133 | 352 to 358 |

wherein, optionally, fragment A contains one or more heterologous modifications.
4. The polypeptide of item 2 or 3 wherein the fragment B comprises an amino acid sequence selected from the group consisting of the amino acid sequences according to the following table and amino acid sequences having an identity of at least 85%, more preferred at least 90%, even more preferred 95%, particularly preferred at least 98%, most preferred at least 99%, with the respective amino acid sequence shown in the following table:

| Sequence | Sequence | | N-terminal | C-terminal |
|---|---|---|---|---|
| based on | according to | | amino acid | amino acid |
| penton base | UniProt | SEQ ID | selected from | selected from |
| protomer of | Acc. No. | NO: | positions | positions |
| hAd3 | Q2Y0H9 | 1 | 441 to 444 | 491 to 494 |
| hAd2 | P03276 | 2 | 468 to 471 | 518 to 521 |
| hAd4 | Q2KSF3 | 3 | 422 to 445 | 465 to 468 |
| hAd5 | P12538 | 4 | 468 to 471 | 491 to 494 |
| hAd7 | Q9JFT6 | 5 | 441 to 444 | 464 to 467 |
| hAd11 | D2DM93 | 6 | 458 to 461 | 481 to 484 |
| hAd12 | P36716 | 7 | 394 to 398 | 418 to 421 |
| hAd17 | F1DT65 | 8 | 414 to 417 | 438 to 441 |
| hAd25 | M0QUK0 | 9 | 441 to 444 | 454 to 457 |
| hAd35 | Q7T941 | 10 | 498 to 501 | 521 to 524 |
| hAd37 | Q912J1 | 11 | 415 to 418 | 438 to 441 |
| hAd41 | F8WQN4 | 12 | 405 to 408 | 438 to 441 |
| gorAd | E5L3Q9 | 13 | 459 to 462 | 482 to 485 |
| ChimpAd | G9G849 | 14 | 421 to 424 | 444 to 457 |
| sAd18 | H8PFZ9 | 15 | 396 to 399 | 419 to 422 |
| sAd20 | F6KSU4 | 16 | 401 to 404 | 424 to 427 |
| sAd49 | F2WTK5 | 17 | 399 to 402 | 422 to 425 |
| rhAd51 | A0A0A1EWW1 | 18 | 395 to 398 | 418 to 421 |
| rhAd52 | A0A0A1EWX7 | 19 | 393 to 396 | 416 to 419 |
| rhAd53 | A0A0A1EWZ7 | 20 | 394 to 397 | 417 to 420 |

wherein, optionally, fragment B contains one or more heterologous modifications.
5. The polypeptide according to any one of items 2 to 4 characterized in that fragment A and/or B contain(s) one or more heterologous modifications wherein said one or more heterologous modifications is/are contained in the following sites:
the RGD loop region of fragment A; and/or
the V-loop of fragment A; and/or
the floor region having the sequence (from N- to C-terminal)

(SEQ ID NO: 21)
X₁-X₂-X₃-X₄-X₅-X₆-D-X₇-X₈-X₉-S-Y-N-X₁₀-X₁₁-X₁₂-
X₁₃-X₁₄-X₁₅-X₁₆

of fragment A, wherein
X₁ is I or L, and is preferably I;
X₂ is selected from the group consisting of K, Q and E, and is preferably Q;
X₃ is P or A, and is preferably P,
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably 5,
X₈ is selected from the group consisting of K, T and S, and is preferably K;
X₉ is selected from the group consisting of K, S, N, G and D, and is preferably S,
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably ID,
X₁₅ is selected from the group consisting of S, K, Q and T, and is preferably K; and
X₁₆ is selected from the group consisting of T, N, I, K and M, and is preferably I; and/or
   the sequence (from N- to C-terminal) T-H-V-F-X₁₇-R-F-P (SEQ ID NO: 22) of fragment B wherein X₁₇ is D or N, and is preferably N.
6. The polypeptide of item 5 wherein the N-terminus of the RGD loop region of fragment A is defined by the following sequence (from N-terminal to C-terminal):

(SEQ ID NO: 23)
X₁₈-X₁₉-X₂₀-X₂₁-X₂₂-X₂₃-X₂₄-X₂₅-X₂₆

wherein
X₁₈ is selected from the group consisting of D, E and N, and is preferably D;
X₁₉ is selected from the group consisting of V, L, and is preferably V;
X₂₀ is any amino acid, preferably selected from the group consisting of A, O, E, K, S, and T, and is more preferably T;
X₂₁ is any amino add; preferably selected from the group consisting of A, D, E, and K, and is more preferably A;
X₂₂ is selected from the group consisting of F, Y, and IN, and is preferably Y;
X₂₃ is selected from the group consisting of A, D, E, N, and Q, is preferably E or Q, and is more preferably E;
X₂₄ is any amino acid, preferably selected from the group consisting of A, O, E, N, and K, and is more preferably E;
X₂₅ is selected from the group consisting of S or T, and is preferably S; and
X₂₆ is any amino acid and constitutes the N-terminal amino acid of the RGD loop region
7. The polypeptide of item 5 or 6 wherein the C-terminus of the RGD loop region of fragment A is defined by the following sequence (from N-terminal to C-terminal):

(SEQ ID NO: 24)
X₂₇-X₂₈-X₂₉-X₃₀-X₃₁-X₃₂-X₃₃-X₃₄

Wherein
X₂₇ is any amino add and constitutes the C-terminal amino acid of the second RGD loop;
X₂₈ is selected from the group consisting of i, L and V, and is preferably I;
X₂₉ is selected from the group consisting of D, E, K, N, Q, and V, is preferably Q or K, and is more preferably Q;
X₃₀ is selected from the group consisting of C, G and P, and is preferably P;
X₃₁ is selected from the group consisting of 1, L and V, is preferably L or V and is more preferably L;
X₃₂ is selected from the group consisting of D, E, S and T, is preferably E or T and is more preferably E;
X₃₃ is selected from the group consisting of D, E, S and T, is preferably E, or T, and is more preferably K; and
X₃₄ is selected from the group consisting of D and E, and is preferably D;
8. The polypeptide according to any one of items 5 to 7 wherein the N-terminus of the V loop of fragment A is defined by the following sequence (from N-terminal to C-terminal):

(SEQ ID NO: 25)
X₃₅-X₃₆-X₃₇-X₃₈-X₃₉-X₄₀-X₄₁-X₄₂

wherein
X₃₅ is selected from the group consisting of F, Y, and W, and is preferably F;
X₃₆ is selected from the croup consisting of H, K and R, and is preferably K;
X₃₇ is selected from the group consisting of A, V, i, and L, and is preferably A;
X₃₆ is selected from the group consisting of H, K, and R, and is preferably R;
X₃₉ is selected from the group consisting of A, V, I, and L, and is preferably V;
X₄₀ is selected from the group consisting of A, V, I, L and M, and is preferably M;
X₄₁ is selected from the group consisting of A, V, I, and L, and is preferably V; and
X₄₂ is any amino acid and constitutes the N-terminal amino acid of the V loop.
9. The polypeptide according to any one of items 5 to 8 wherein the C-terminus of the V loop of fragment A is defined by the following sequence (from N-terminal to C-terminal):

(SEQ ID NO: 26)
X₄₃-X₄₄-X₄₅-X₄₆-X₄₇-X₄₈-X₄₉

wherein
X₄₃ is any amino acid and constitutes the C-terminal amino acid of the V loop;
X₄₄ is selected from the group consisting of F, Y, and W, and is preferably Y;
X₄₅ is selected from the group consisting of D, E, S and T, is preferably E or T and is more preferably E;
X₄₆ is selected from the group consisting of F, Y, and W, and is preferably W;
X₄₇ is selected from the group consisting of A, F, V, Y, and W, is preferably F or V and is more preferably F;
X₄₈ is selected from the group consisting of D, E, S and T, is preferably D or E and is more preferably E; and
X₄₉ is selected from the group consisting of F, Y, and VV, and is preferably F.
10. The polypeptide according to any one of items 2 to 9 wherein the heterologous modification is selected from the group consisting of one or more single amino acid mutations in comparison to the wildtype sequence of fragment A and/or B, one or more replacements of wildtype amino acid stretches by one or more heterologous amino acids and/or amino acid stretches one or more insertions of heterologous amino acid stretches, one or more deletions of one or more amino acids and one or more amino acid modifications as well as any combination(s) thereof.
11. The polypeptide according to any one of items 2 to 10, wherein the heterologous modification provides a target specific binding entity.
12. The polypeptide of item 11, wherein the target specific binding entity is selected from the group consisting of antigens, epitopes, CDRs, antibodies, antibody fragments such as an antigen binding (Fab) fragment, a Fab′ fragment, a F(ab′)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, a paratope, an alternative scaffold protein, and a fusion protein thereof, a toxin and a venom.
13. The polypeptide according to any one of items 1 to 3 having one of the following amino acid sequences (from N-terminal to C-terminal):

(SEQ ID NO: 27)
MSYYHHHHHHDYDIPTTENLYFQGAMGSGIQPNVNEYMFSNKFKARVMVS
RKAPEGVTVNDTYDHKEDILKYEWFEFILPEGNFSATMTIDLMNNAIIDN
YLEIGRQNGVLESDIGVKFDTRNFRLGWDPETKLIMPGVYTYEAFHPDIV
LLPGCGVDFTESRLSNLLGIRKRHPFQEGFKIMYEDLEGGNIPALLDVTA
YEESKKDTTTETTTKKELKIQPLEKDSKSRSYNVLEDKINTAYRSWYLSY
NYGNPEKGIRSWTLLTTSDVTCGANGDSGNPVFSKSFYNEQAVYSQQLRQ
ATSLTHVFNRFPENQILIRPPAPTITTVSENVP
(SEQ ID NO: 32)
GAMGSGIQPNVNEYMFSNKFKARVMVSRKAPEGVTVNDTYDHKEDILKYE
WFEFILPEGNFSATMTIDLMNNAIIDNYLEIGRQNGVLESDIGVKFDTRN
FRLGWDPETKLIMPGVYTYEAFHPDIVLLPGCGVDFTESRLSNLLGIRKR
HPFQEGFKIMYEDLEGGNIPALLDVTAYEESKKDTTTETTTKKELKIQPL
EKDSKSRSYNVLEDKINTAYRSWYLSYNYGNPEKGIRSWTLLTTSDVTCG
ANGDSGNPVFSKSFYNEQAVYSQQLRQATSLTHVFNRFPENQILIRPPAP
TITTVSENVP
(SEQ ID NO: 28)
MSYYHHHHHHDYDIPTTENLYFQGTIMHTNMPNVNEFMYSNKFKARVMVS
RKAPEGVTVNDTYDHKEDILEYEWVEFELPEGNFSVTMTIDLMNNAIIDN
YLAVGRQNGVLESDIGVKFDTRNFRLGWDPVTELVMPGVYTNEAFHPDIV
LLPGCGVDFTESRLSNLLGIRKRQPFQEGFQIMYEDLEGGNIPALLDVDA
YEKSKKDTTTETTTKKELKIQPVEKDSKDRSYNVLPDKINTAYRSWYLAY
NYGDPEKGVRSWTLLTTSDVTCGVEQAELLPVYSKSFFNEQAVYSQQLRA
FTSLTHVFNRFPENQILVRPPAPTITTVSENVP
(SEQ ID NO: 33)
QGTIMHTNMPNVNEFMYSNKFKARVMVSRKAPEGVTVNDTYDHKEDILEY
EWVEFELPEGNFSVTMTIDLMNNAIIDNYLAVGRQNGVLESDIGVKFDTR
NFRLGWDPVTELVMPGVYTNEAFHPDIVLLPGCGVDFTESRLSNLLGIRK
RQPFQEGFQIMYEDLEGGNIPALLDVDAYEKSKKDTTTETTTKKELKIQP
VEKDSKDRSYNVLPDKINTAYRSWYLAYNYGDPEKGVRSWTLLTTSDVTC
GVEQAELLPVYSKSFFNEQAVYSQQLRAFTSLTHVFNRFPENQILVRPPA
PTITTVSENVP

14. An isolated engineered polypeptide comprising the large fragment of the alpha-helical domain of an adenovirus penton base protein which polypeptide lacks the small fragment of the alpha-helical domain and the jellyroll fold domain of the adenovirus penton base protein, wherein said large fragment optionally contains one or more heterologous modifications.
15. The polypeptide of item 14 having, from N- to C-terminal, the structure of the following general formula II:

N-A-C (II)
wherein
A represents an amino acid stretch corresponding to the N-terminal amino acid stretch of the adenovirus penton base present between the first and the second amino acid stretch forming the jellyroll fold domain of the adenovirus penton base;
N may or may not be present, and, if present, represents a chemical group consisting of an amino acid, an oligopeptide and a polypeptide;
C: may or may not be present, and, if present, represents a chemical group consisting of an amino acid, an oligopeptide and a polypeptide;
wherein, optionally, fragment A contain one or more heterologous modifications.
16. The polypeptide of item 15 wherein the fragment A comprises an amino acid sequence selected from the group consisting of the amino acid sequences according to the following table and amino acid sequences having an identity of at least 85%, more preferred at least 90%, even more preferred 95%, particularly preferred at least 98%, most preferred at least 99%, with the respective amino acid sequence shown in the following table:

| Sequence | Sequence | | N-terminal | C-terminal |
|---|---|---|---|---|
| based on | according to | | amino acid | amino acid |
| penton base | UniProt | SEQ ID | selected from | selected from |
| protomer of | Acc. No. | NO: | positions | positions |
| hAd3 | Q2Y0H9 | 1 | 130 to 137 | 399 to 405 |
| hAd2 | P03276 | 2 | 130 to 137 | 426 to 432 |
| hAd4 | Q2KSF3 | 3 | 126 to 133 | 380 to 386 |
| hAd5 | P12538 | 4 | 130 to 137 | 426 to 432 |
| hAd7 | Q9JFT6 | 5 | 130 to 137 | 399 to 405 |
| hAd11 | D2DM93 | 6 | 130 to 137 | 416 to 422 |
| hAd12 | P36716 | 7 | 120 to 127 | 352 to 358 |
| hAd17 | F1DT65 | 8 | 117 to 124 | 371 to 377 |
| hAd25 | M0QUK0 | 9 | 125 to 133 | 389 to 395 |
| hAd35 | Q7T941 | 10 | 131 to 138 | 446 to 452 |
| hAd37 | Q912J1 | 11 | 117 to 124 | 373 to 379 |
| hAd41 | F8WQN4 | 12 | 128 to 135 | 362 to 368 |
| gorAd | E5L3Q9 | 13 | 131 to 138 | 417 to 423 |
| ChimpAd | G9G849 | 14 | 126 to 133 | 373 to 379 |
| sAd18 | H8PFZ9 | 15 | 128 to 135 | 354 to 360 |
| sAd20 | F6KSU4 | 16 | 127 to 134 | 359 to 365 |
| sAd49 | F2WTK5 | 17 | 128 to 135 | 357 to 363 |
| rhAd51 | A0A0A1EWW1 | 18 | 125 to 132 | 353 to 359 |
| rhAd52 | A0A0A1EWX7 | 19 | 125 to 132 | 351 to 357 |
| rhAd53 | A0A0A1EWZ7 | 20 | 126 to 133 | 352 to 358 |

wherein, optionally, fragment A contains one or more heterologous modifications.
17. The polypeptide of item 15 or 16 characterized in that fragment A contains one or more heterologous modifications wherein said one or more heterologous modifications is/are contained in the following sites:
   the RGD loop region of fragment A; and/or
   the V-loop of fragment A; and/or
   the floor region having the sequence (from N- to C-terminal)

(SEQ ID NO: 21)
X₁-X₂-X₃-X₄-X₅-X₆-D-X₇-X₈-X₉-S-Y-N-X₁₀-X₁₁-X₁₂-
X₁₃-X₁₄-X₁₅-X₁₆

of fragment A, wherein
X₁ is I or L, and is preferably I;
X₂ is selected from the group consisting of K, Q and E, and is preferably Q;
X₃ is P or A, and is preferably P,
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably S;
X₈ is selected from the group consisting of K, T and S, and is preferably K;
X₉ is selected from the group consisting of K, S, N, G and D, and is preferably S;
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably ID,
X₁₅ is selected from the group consisting of S, K, Q and T, and is preferably K; and
X₁₆ is selected from the group consisting of T, N, I, K and M, and is preferably I; and/or
the sequence (from N- to C-terminal) T-H-V-F-X₁₇-R-F-P (SEQ ID NO: 22) of fragment B wherein X₁₇ is D or N, and is preferably N.
18. The polypeptide of item 17 wherein the N-terminus of the RGD loop region of fragment A is defined by the following sequence (from N-terminal to C-terminal):

(SEQ ID NO: 23)
X₁₈-X₁₉-X₂₀-X₂₁-X₂₂-X₂₃-X₂₄-X₂₅-X₂₆

wherein)
X₁₈ is selected from the group consisting of D, E and N, and is preferably D;
X₁₉ is selected from the group consisting of V, L, and is preferably V;
X₂₀ is any amino acid, preferably selected from the group consisting of A, D, E, K, S, and T, and is more preferably T;
X₂₁ is any amino add, preferably selected from the group consisting of A, D, E, and K, and is more preferably A;
X₂₂ is selected from the group consisting of F, Y, and W, and is preferably Y;
X₂₃ is selected from the group consisting of A, D, E, N, and Q, is preferably E or Q, and is more preferably E;
X₂₄ is any amino acid, preferably selected from the group consisting of A, D, E, N, and K, and is more preferably E,
X₂₅ is selected from the group consisting of S or T, and is preferably S; and
X₂₆ is any amino acid and constitutes the N-terminal amino add of the RGD loop region
19. The polypeptide of item 17 or 18 wherein the C-terminus of the RGD loop region of fragment A is defined by the following sequence (from N-terminal to C-terminal):

(SEQ ID NO: 24)
X₂₇-X₂₈-X₂₉-X₃₀-X₃₁-X₃₂-X₃₃-X₃₄

Wherein
X₂₇ is any amino add and constitutes the C-terminal amino add of the second RGD loop;
X₂₈ is selected from the group consisting of I, L and V, and is preferably I;
X₂₉ is selected from the group consisting of D, E, K, N, Q, and V, is preferably or K, and is more preferably Q;
X₃₀ is selected from the group consisting of C, G and P, and is preferably P;
X₃₁ is selected from the group consisting of I, L and V, is preferably L or V and is more preferably L;
X₃₂ is selected from the group consisting of D, E, S and T, is preferably E or T and is more preferably E;
X₃₃ is selected from the group consisting of D, E, S and T, is preferably E, or T, and is more preferably K; and
X₃₄ is selected from the group consisting of 0 and E, and is preferably D;
20. The polypeptide according to any one of items 17 to 19 wherein the N-terminus of the V loop of fragment A is defined by the following sequence (from N-terminal to C-terminal):

(SEQ ID NO: 25)
X₃₅-X₃₆-X₃₇-X₃₈-X₃₉-X₄₀-X₄₁-X₄₂

wherein
X₃₅ is selected from the group consisting of F, Y, and W, and is preferably F;
X₃₆ is selected from the group consisting of H, K and R, and is preferably K;
X₃₇ is selected from the croup consisting of A, V, I, and L, and is preferably A;
X₃₈ is selected from the group consisting of H, K, and R, and is preferably R;
X₃₉ is selected from the group consisting of A, V, I, and L, and is preferably V;
X₄₀ is selected from the group consisting of A, V, I, L and M, and is preferably M;
X₄₁ is selected from the group consisting of A, V, I, and L, and is preferably V; and
X₄₂ is any amino acid and constitutes the N-terminal amino acid of the V loop.
21. The polypeptide according to any one of items 17 to 20 wherein the C-terminus of the V loop of fragment A is defined by the following sequence (from N-terminal to C-terminal):

(SEQ ID NO: 26)
X₄₃-X₄₄-X₄₅-X₄₆-X₄₇-X₄₈-X₄₉

wherein
X₄₃ is any amino acid and constitutes the C-terminal amino acid of the V loop;
X₄₄ is selected from the group consisting of F, Y, and W, and is preferably Y;
X₄₅ is selected from the group consisting of D, E, S and T, is preferably E or T and is more preferably E;
X₄₆ is selected from the group consisting of F, Y, and W, and is preferably W;
X₄₇ is selected from the group consisting of A, F, V, Y, and W, is preferably F or V and is more preferably F;
X₄₈ is selected from the group consisting of D, E, S and T, is preferably D or E and is more preferably E; and
X₄₉ is selected from the group consisting of F, Y, and IN, and is preferably F.
22. The polypeptide according to any one of items 15 to 21 wherein the heterologous modification is selected from the group consisting of one or more single amino acid mutations in comparison to the wildtype sequence of fragment A, one or more replacements of wildtype amino acid stretches by one or more heterologous amino acids and/or amino acid stretches one or more insertions of heterologous amino acid stretches, one or more deletions of one or more amino acids and one or more amino acid modifications as well as any combination(s) thereof.
23. The polypeptide according to any one of items 15 to 22, wherein the heterologous modification provides a target specific binding entity.
24. The polypeptide of item 23, wherein the target specific binding entity is selected from the group consisting of antigens, epitopes, CDRs, antibodies, antibody fragments such as an antigen binding (Fab) fragment, a Fab′ fragment, a F(ab″)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, a paratope, an alternative scaffold protein, and a fusion protein thereof, a toxin and a venom.
25. A nucleic acid encoding a polypeptide according to any one of the preceding items.
26. A vector comprising the nucleic acid of item 25.
27. The vector of item 26 containing the nucleic acid of item 15 within an expression cassette.
28. A recombinant host cell comprising the nucleic acid of item 24 or the vector of item 26 or 27.
29. A method for the production of a polypeptide according to any one of items 1 to 24 comprising the step of culturing the host cell of item 28 containing the vector of item 28 under conditions allowing the expression of said polypeptide.
30. The method of item 29 further comprising the step of purifying the polypeptide from the cultured host cells.
31. An engineered adenovirus penton base protein comprising the polypeptide according to any one of items 5 to 12 fused to the multimerization domain (jellyroll fold domain) of an adenovirus penton base protein.
32. The penton base protein of item 31 having, from N- to C-terminal, the structure of the following general formula III:

D-A-E-B-F (III)
wherein A and B are the fragments of the alpha-helical crown domain as defined in any one of items 2 to 4, and D, E and F are the amino acid sequences of an adenovirus penton base forming the multimerization (jellyroll fold) domain, wherein one or more heterologous modifications is/are present in the floor region of fragment A and/or in the B loop of fragment B.
33. An engineered adenovirus penton base protein comprising the polypeptide according to any one of item 15 to 24 fused to the multimerization domain (jellyroll fold domain) of an adenovirus penton base protein.
34. The penton base of item 33 having, from N- to C-terminal, the structure of the following general formula IV:

D-A-E-Li-F (IV)
wherein A is the large fragment of the alpha-helical crown domain as defined in any one of items 15 or 16, and D, E and F are the amino acid sequences of an adenovirus penton base forming the multimerization (jellyroll fold) domain, wherein, optionally and preferably, one or more heterologous modifications is/are present in the floor region of fragment A, and wherein Li is a linker selected from peptides, oligopeptides, polypeptides, proteins and protein complexes.
35. A penton base of item 32 or 34 wherein fragment D of general formula (III) or (IV) has the following consensus sequence (SEQ ID NO: 34):

(U)₁₋₄₇ PTJ₁GRNSIRY SJ₂J₃x₄PJ₅J₆DTT J₇J_{B}YLVDNKSA DIASLNYQND HSNFJ₅TTVJ₉Q NNDJ₁₀J₁₁PJ₁₂EAJ₁₃ TQT INJ₁₄DJ₁₅RS RWGJ₁₆L-T₁₇LKTIJ₁₈ J₁₉TZ₁Z₂Z₃Z₄Z₅Z₆Z₇Z₈ Z₉Z₁₀Z₁₁Z₁₂Z₁₃Z₁₄Z₁₅
wherein: fragment D ends on the C-terminal side before Z₁ at residue T or at an amino acid from Z₁ to Z₁₅
   U is any or no amino acid
   J₁ is E or G
   J₂ is E or S
   J₃ is L or V
   J₄ is A or S
   J₅ is L or Q
   J₆ is Y or E
   J₇ is R or K
   J₈ is V or L
   J₉ is V or I
   J₁₀ is F or Y
   J₁₁ is T or S
   J₁₂ is A or T or I or G
   J₁₃ is S or G
   J₁₄ is F or L
   J₁₅ is E or D
   J₁₆ is A or G
   J₁₇ is D or Q
   J₁₈ is L or M
   J₁₉ is H or R
   Z₁, if present, is N
   Z₂, if present, is M
   Z₃, if present, is P
   Z₄, if present, is N
   Z₅, if present, is V or I
   Z₆, if present, is N
   Z₇, if present, is E or D
   Z₈, if present, is Y or F
   Z₉, if present, is M
   Z₁₀, if present, is F or S or Y
   Z₁₁, if present, is T or S
   Z₁₂, if present, is S or N
   Z₁₃, if present, is K
   Z₁₄, if present, is F
   Z₁₆, if present, is K.
36. The penton base of item 35 wherein the fragment D comprises an amino acid sequence selected from the group consisting of the amino acid sequences of the following table and amino acid sequences having and identity of at least 85%, more preferred at least 90%, even more preferred 95%, particularly preferred at least 98%, most preferred at least 99%, with the respective amino acid sequence shown in the following table:

| Sequence | Sequence | | N-terminal | C-terminal |
|---|---|---|---|---|
| based on | according to | | amino acid | amino acid |
| penton base | UniProt | SEQ ID | selected from | selected from |
| protomer of | Acc. No. | NO: | positions | positions |
| hAd3 | Q2Y0H9 | 1 | 1 to 48 | 129 to 144 |
| hAd2 | P03276 | 2 | 1 to 48 | 129 to 144 |
| hAd4 | Q2KSF3 | 3 | 1 to 44 | 125 to 140 |
| hAd5 | P12538 | 4 | 1 to 48 | 129 to 144 |
| hAd7 | Q9JFT6 | 5 | 1 to 48 | 129 to 144 |
| hAd11 | D2DM93 | 6 | 1 to 48 | 129 to 144 |
| hAd12 | P36716 | 7 | 1 to 38 | 119 to 134 |
| hAd17 | F1DT65 | 8 | 1 to 35 | 116 to 131 |
| hAd25 | M0QUK0 | 9 | 1 to 43 | 124 to 139 |
| hAd35 | Q7T941 | 10 | 1 to 49 | 130 to 145 |
| hAd37 | Q912J1 | 11 | 1 to 35 | 116 to 131 |
| hAd41 | F8WQN4 | 12 | 1 to 46 | 127 to 142 |
| gorAd | E5L3Q9 | 13 | 1 to 49 | 130 to 145 |
| ChimpAd | G9G849 | 14 | 1 to 44 | 125 to 140 |
| sAd18 | H8PFZ9 | 15 | 1 to 46 | 127 to 142 |
| sAd20 | F6KSU4 | 16 | 1 to 45 | 126 to 141 |
| sAd49 | F2WTK5 | 17 | 1 to 48 | 127 to 142 |
| rhAd51 | A0A0A1EWW1 | 18 | 1 to 43 | 124 to 139 |
| rhAd52 | A0A0A1EWX7 | 19 | 1 to 43 | 124 to 139 |
| rhAd53 | A0A0A1EWZ7 | 20 | 1 to 44 | 125 to 140 |

37. The penton base according to any of items 32 to 36 fragment E of general formula (III) or (IV) has the following sequence (SEQ ID NO: 35):

Z₁₇Z₁₈Z₁₉Z₂₀Z₂₁Z₂₂Z₂₃Z₂₄Z₂₅Z₂₆ Z₂₇QVYWSLPDJ₂₀ MJ₂₁DPVTFRST J₂₂QJ₂₃J₂₄NJ₂₅PVVGJ₂₆ ELZ₂₈Z₂₉Z₃₀
wherein: fragment E begins on the N-terminal side at an amino acid from Z₁₇ to Z₂₇ or at amino acid Q after Z₂₇,
amino acid stretch B ends on the C-terminal side before Z₂₈ at amino acid L or at an amino acid from Z₂₈ to Z₃₀;
   Z₁₇, if present, is L or S
   Z₁₈, if present, is T or P or C
   Z₁₉, if present, is T or P
   Z₂₀, if present, is P or S or A or R
   Z₂₁, if present, is N or D
   Z₂₂, if present, is G or V
   Z₂₃, if present, is H or T
   Z₂₄, if present, is C
   Z₂₅, if present, is G
   Z₂₈, if present, is A or V or S
   Z₂₇, if present, is E or Q
   J₂₀ is L or M
   J₂₁ is Q or K
   J₂₂ is Q or R or S
   J₂₃ is V or I
   J₂₄ is S or N
   J₂₅ is Y or F
   J₂₈ is A or V
   Z₂₈, if present, is M or L
   Z₂₉, if present, is P
   Z₃₀, if present, is V or F.
38. The penton base of item 37 wherein the fragment E comprises an amino acid sequence selected from the group consisting of the amino acid sequences of the following table and amino acid sequences having and identity of at least 85%, more preferred at least 90%, even more preferred 95%, particularly preferred at least 98%, most preferred at least 99%, with the respective amino acid sequence shown in the following table:

| Sequence | Sequence | | N-terminal | C-terminal |
|---|---|---|---|---|
| based on | according to | | amino acid | amino acid |
| penton base | UniProt | SEQ ID | selected from | selected from |
| protomer of | Acc. No. | NO: | positions | positions |
| hAd3 | Q2Y0H9 | 1 | 398 to 409 | 440 to 443 |
| hAd2 | P03276 | 2 | 425 to 436 | 467 to 470 |
| hAd4 | Q2KSF3 | 3 | 379 to 390 | 421 to 444 |
| hAd5 | P12538 | 4 | 425 to 436 | 467 to 470 |
| hAd7 | Q9JFT6 | 5 | 398 to 409 | 440 to 443 |
| hAd11 | D2DM93 | 6 | 415 to 426 | 457 to 460 |
| hAd12 | P36716 | 7 | 351 to 362 | 393 to 397 |
| hAd17 | F1DT65 | 8 | 370 to 381 | 413 to 416 |
| hAd25 | M0QUK0 | 9 | 388 to 399 | 440 to 443 |
| hAd35 | Q7T941 | 10 | 445 to 456 | 497 to 500 |
| hAd37 | Q912J1 | 11 | 372 to 383 | 414 to 417 |
| hAd41 | F8WQN4 | 12 | 362 to 373 | 404 to 407 |
| gorAd | E5L3Q9 | 13 | 416 to 427 | 458 to 461 |
| ChimpAd | G9G849 | 14 | 372 to 383 | 420 to 423 |
| sAd18 | H8PFZ9 | 15 | 353 to 364 | 395 to 398 |
| sAd20 | F6KSU4 | 16 | 358 to 369 | 400 to 403 |
| sAd49 | F2WTK5 | 17 | 356 to 367 | 398 to 401 |
| rhAd51 | A0A0A1EWW1 | 18 | 352 to 363 | 394 to 397 |
| rhAd52 | A0A0A1EWX7 | 19 | 350 to 361 | 392 to 395 |
| rhAd53 | A0A0A1EWZ7 | 20 | 351 to 362 | 393 to 396 |

39. The penton base according to any one of items 32 to 38 wherein fragment F of general formula (III) or (IV) has the following sequence (SEQ ID NO: 36):

Z₃₁Z₃₂Z₃₃ALTDHGT LPLRSSIJ₂₇GV QRVTJ₂₈TDARR RTCPYVYKA LGIVJ₃₀PJ₃₁VLS SRTF
wherein: fragment F begins on the N-terminal side at an amino acid from Z₃₁ to Z₃₃ or at amino acid A after Z₃₃;
   Z₃₁, if present, is N
   Z₃₂, if present, is V
   Z₃₃, if present, is P
   J₂₇ is R or S or G
   J₂₈ is V or I
   J₂₉ is Y or H
   J₃₀ is A or S
   J₃₁ is R or K
40. The penton base of item 39 wherein fragment F comprises an amino acid sequence selected from the group consisting of the amino acid sequences of the following table and amino acid sequences having and identity of at least 85%, more preferred at least 90%, even more preferred 95%, particularly preferred at least 98%, most preferred at least 99%, with the respective amino acid sequence shown in the following table:

| Sequence | Sequence | | N-terminal | |
|---|---|---|---|---|
| based on | according to | | amino acid | C-terminal |
| penton base | UniProt | SEQ ID | selected from | amino acid |
| protomer of | Acc. No. | NO: | positions | position |
| hAd3 | Q2Y0H9 | 1 | 492 to 495 | 544 |
| hAd2 | P03276 | 2 | 519 to 522 | 571 |
| hAd4 | Q2KSF3 | 3 | 466 to 469 | 535 |
| hAd5 | P12538 | 4 | 492 to 495 | 571 |
| hAd7 | Q9JFT6 | 5 | 465 to 468 | 544 |
| hAd11 | D2DM93 | 6 | 482 to 485 | 561 |
| hAd12 | P36716 | 7 | 419 to 422 | 497 |
| hAd17 | F1DT65 | 8 | 438 to 441 | 517 |
| hAd25 | M0QUK0 | 9 | 455 to 458 | 534 |
| hAd35 | Q7T941 | 10 | 522 to 525 | 561 |
| hAd37 | Q912J1 | 11 | 439 to 442 | 519 |
| hAd41 | F8WQN4 | 12 | 439 to 432 | 508 |
| gorAd | E5L3Q9 | 13 | 483 to 486 | 875 |
| ChimpAd | G9G849 | 14 | 445 to 458 | 532 |
| sAd18 | H8PFZ9 | 15 | 420 to 423 | 508 |
| sAd20 | F6KSU4 | 16 | 425 to 428 | 512 |
| sAd49 | F2WTK5 | 17 | 423 to 426 | 511 |
| rhAd51 | A0A0A1EWW1 | 18 | 419 to 422 | 505 |
| rhAd52 | A0A0A1EWX7 | 19 | 417 to 420 | 503 |
| rhAd53 | A0A0A1EWZ7 | 20 | 418 to 421 | 504 |

41. A pentameric complex of the engineered adenovirus penton base protein according to any one of items 32 to 40.
42. A virus-like particle (VLP) comprising 12 pentameric complexes of item 41.
43. The polypeptide according to any one of items 1 to 24 or the VLP of item 42 for use as a medicament.
44. A pharmaceutical composition comprising a polypeptide according to any one of items 1 to 24 or the engineered adenovirus penton base protein according to any one of items 32 to 40 or the VLP of item 42, optionally together with at least one pharmaceutically acceptable carrier, excipient and/or diluent.
45. A method for producing the VLP of item 42 comprising the step of incubating a solution of a penton base according to any one of items 32 to 40 under conditions allowing the assembly of the polypeptide into a VLP.
46. The polypeptide according to any one of items 1 to 24 or the VLP of item 42 for use in the treatment and/or prevention of an infectious disease, an immune disease, tumor or cancer.
47. A method of identifying a binding sequence to a target molecule comprising the steps of:
   (ia) preparing a library of vectors each containing a nucleotide sequence encoding a polypeptide having a candidate binding sequence in an expression cassette, each polypeptide encoded by said nucleotide sequence comprising a candidate binding sequence as a heterologous modification in one or more of RGD loop region and/or V loop and/or floor region and/or B loop as defined in any one of items 5 to 9, wherein the candidate binding sequence encoded by the nucleotide sequence in each vector is different such that the vectors contain a randomized library of nucleotide sequences encoding randomized candidate binding sequences; or
   (ib) preparing a library of vectors each containing a nucleotide sequence encoding a polypeptide having a candidate binding sequence in an expression cassette, each polypeptide encoded by said nucleotide sequence comprising a candidate binding sequence as a heterologous modification in one or more of RGD loop region and/or V loop and/or floor region as defined in any one of items 17 to 21, wherein the candidate binding sequence encoded by the nucleotide sequence in each vector is different such that the vectors contain a randomized library of nucleotide sequences encoding randomized candidate binding sequences
   (ii) expressing the polypeptides encoded by the nucleotide sequences from the library of vectors of step (ia) or ib) in a host cell or a cell-free system, preferably cell free system;
   (iii) contacting the polypeptides expressed in step (ii), optionally after purification from the host cells or the cell-free system, preferably cell free system, with the target molecule; and
   (iv) detecting which polypeptide(s) have/has bound to the target molecule.
48. The method of item 47 further comprising the step of determining the dissociation constant(s) (K_{d}) of the polypeptide(s) bound to the target molecule.

1. An isolated engineered polypeptide comprising the amino acid stretches essentially corresponding to a first and a second fragment of the penton base protein of an adenovirus wherein the first fragment of the polypeptide is present between the first and second amino acid stretches forming the jellyroll fold domain in the full length penton base protein and wherein the second fragment of the polypeptide is present between the second and third fragments forming the jellyroll fold domain in the full length penton base, respectively, wherein the isolated engineered polypeptide lacks the amino acid stretches forming the jellyroll fold domain of the adenovirus penton base, wherein optionally the first and/or second fragments of the polypeptide contain(s) one or more heterologous modification(s).

2. The polypeptide of claim 1 having the structure of the following general formula (I):
N-A-L-B-C (I)
wherein
A represents an amino acid stretch corresponding to the N-terminal amino acid stretch of the adenovirus penton base protein present between the first and the second amino acid stretch forming the jellyroll fold domain of the adenovirus penton base;
B represents an amino acid stretch corresponding to the C-terminal amino acid stretch of the adenovirus penton base protein inserted between the second and the third amino acid stretch forming the jellyroll fold domain of the adenovirus penton base protein;
L represents a chemical group selected from the group consisting of an amino acid, an oligopeptide and a polypeptide;
N may or may not be present, and, if present, represents a chemical group consisting of an amino acid, an oligopeptide and a polypeptide;
C: may or may not be present, and, if present, represents a chemical group consisting of an amino acid, an oligopeptide and a polypeptide;
wherein, optionally, fragment A and/or B contain(s) one or more heterologous modifications.

3. The polypeptide of claim 2 wherein the fragment A comprises an amino acid sequence selected from the group consisting of the amino acid sequences according to the following table and amino acid sequences having an identity of at least 85% with the respective amino acid sequence shown in the following table:

| | | | N-terminal | C-terminal |
|---|---|---|---|---|
| Sequence based | Sequence | | amino acid | amino acid |
| on penton base | according to | SEQ ID | selected from | selected from |
| protomer of | UniProt Acc. No. | NO: | positions | positions |
| hAd3 | Q2Y0H9 | 1 | 130 to 137 | 399 to 405 |
| hAd2 | P03276 | 2 | 130 to 137 | 426 to 432 |
| hAd4 | Q2KSF3 | 3 | 126 to 133 | 380 to 386 |
| hAd5 | P12538 | 4 | 130 to 137 | 426 to 432 |
| hAd7 | Q9JFT6 | 5 | 130 to 137 | 399 to 405 |
| hAd11 | D2DM93 | 6 | 130 to 137 | 416 to 422 |
| hAd12 | P36716 | 7 | 120 to 127 | 352 to 358 |
| hAd17 | F1DT65 | 8 | 117 to 124 | 371 to 377 |
| hAd25 | M0QUK0 | 9 | 125 to 133 | 389 to 395 |
| hAd35 | Q7T941 | 10 | 131 to 138 | 446 to 452 |
| hAd37 | Q912J1 | 11 | 117 to 124 | 373 to 379 |
| hAd41 | F8WQN4 | 12 | 128 to 135 | 362 to 368 |
| gorAd | E5L3Q9 | 13 | 131 to 138 | 417 to 423 |
| ChimpAd | G9G849 | 14 | 126 to 133 | 373 to 379 |
| sAd18 | H8PFZ9 | 15 | 128 to 135 | 354 to 360 |
| sAd20 | F6KSU4 | 16 | 127 to 134 | 359 to 365 |
| sAd49 | F2WTK5 | 17 | 128 to 135 | 357 to 363 |
| rhAd51 | A0A0A1EWW1 | 18 | 125 to 132 | 353 to 359 |
| rhAd52 | A0A0A1EWX7 | 19 | 125 to 132 | 351 to 357 |
| rhAd53 | A0A0A1EWZ7 | 20 | 126 to 133 | 352 to 358 |

wherein, optionally, fragment A contains one or more heterologous modifications.

4. The polypeptide of claim 2 wherein the fragment B comprises an amino acid sequence selected from the group consisting of the amino acid sequences according to the following table and amino acid sequences having an identity of at least 85%, more preferred at least 90%, even more preferred 95%, particularly preferred at least 98%, most preferred at least 99%, with the respective amino acid sequence shown in the following table:

| Sequence | Sequence | | N-terminal | |
|---|---|---|---|---|
| based on | according to | | amino acid | C-terminal amino |
| penton base | UniProt | SEQ ID | selected from | acid selected |
| protomer of | Acc. No. | NO: | positions | from positions |
| hAd3 | Q2Y0H9 | 1 | 441 to 444 | 491 to 494 |
| hAd2 | P03276 | 2 | 468 to 471 | 518 to 521 |
| hAd4 | Q2KSF3 | 3 | 422 to 445 | 465 to 468 |
| hAd5 | P12538 | 4 | 468 to 471 | 491 to 494 |
| hAd7 | Q9JFT6 | 5 | 441 to 444 | 464 to 467 |
| hAd11 | D2DM93 | 6 | 458 to 461 | 481 to 484 |
| hAd12 | P36716 | 7 | 394 to 398 | 418 to 421 |
| hAd17 | F1DT65 | 8 | 414 to 417 | 438 to 441 |
| hAd25 | M0QUK0 | 9 | 441 to 444 | 454 to 457 |
| hAd35 | Q7T941 | 10 | 498 to 501 | 521 to 524 |
| hAd37 | Q912J1 | 11 | 415 to 418 | 438 to 441 |
| hAd41 | F8WQN4 | 12 | 405 to 408 | 438 to 441 |
| gorAd | E5L3Q9 | 13 | 459 to 462 | 482 to 485 |
| ChimpAd | G9G849 | 14 | 421 to 424 | 444 to 457 |
| sAd18 | H8PFZ9 | 15 | 396 to 399 | 419 to 422 |
| sAd20 | F6KSU4 | 16 | 401 to 404 | 424 to 427 |
| sAd49 | F2WTK5 | 17 | 399 to 402 | 422 to 425 |
| rhAd51 | A0A0A1EWW1 | 18 | 395 to 398 | 418 to 421 |
| rhAd52 | A0A0A1EWX7 | 19 | 393 to 396 | 416 to 419 |
| rhAd53 | A0A0A1EWZ7 | 20 | 394 to 397 | 417 to 420 |

wherein, optionally, fragment B contains one or more heterologous modifications.

5. The polypeptide of claim 2 wherein fragment A and/or B contain(s) one or more heterologous modifications wherein said one or more heterologous modifications is/are contained in the following sites:
the RGD loop region of fragment A; and/or
the V-loop of fragment A; and/or
the floor region having the sequence (from N- to C-terminal)
(SEQ ID NO: 21)
X₁-X₂-X₃-X₄-X₅-X₆-D-X₇-X₈-X₉-S-Y-
N-X₁₀-X₁₁-X₁₂-X₁₃-X₁₄-X₁₅-X₁₆
of fragment A, wherein
X₁ is I or L, and is preferably I;
X₂ is selected from the group consisting of K, Q and E, and is preferably Q;
X₃ is P or A, and is preferably P;
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably S;
X₈ is selected from the group consisting of K, T and S, and is preferably K;
X₉ is selected from the group consisting of K, S, N, G and D, and is preferably S;
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably D;
X₁₅ is selected from the group consisting of S, K, Q and T, and is preferably K; and
X₁₆ is selected from the group consisting of T, N, I, K and M, and is preferably I; and/or
the sequence (from N- to C-terminal) T-H-V-F-X₁₇-R-F-P (SEQ ID NO: 22) of fragment B wherein X₁₇ is D or N, and is preferably N.

6. The polypeptide of claim 5 wherein the N-terminus of the RGD loop region of fragment A is defined by the following sequence (from N-terminal to C-terminal):
(SEQ ID NO: 23)
X₁₈-X₁₉-X₂₀-X₂₁-X₂₂-X₂₃-X₂₄-X₂₅-X₂₆
wherein
X₁₈ is selected from the group consisting of D, E and N, and is preferably D;
X₁₉ is selected from the group consisting of V, L, and I, and is preferably V;
X₂₀ is any amino acid, preferably selected from the group consisting of A, D, E, K, S, and T, and is more preferably T;
X₂₁ is any amino acid, preferably selected from the group consisting of A, D, E, and K, and is more preferably A;
X₂₂ is selected from the group consisting of F, Y, and W, and is preferably Y;
X₂₃ is selected from the group consisting of A, D, E, N, and Q, is preferably E or Q, and is more preferably E;
X₂₄ is any amino acid, preferably selected from the group consisting of A, D, E, N, and K, and is more preferably E;
X₂₅ is selected from the group consisting of S or T, and is preferably S; and
X₂₆ is any amino acid and constitutes the N-terminal amino acid of the RGD loop region

7. The polypeptide of claim 5 wherein the C-terminus of the RGD loop region of fragment A is defined by the following sequence (from N-terminal to C-terminal):
(SEQ ID NO: 24)
X₂₇-X₂₈-X₂₉-X₃₀-X₃₁-X₃₂-X₃₃-X₃₄
Wherein
X₂₇ is any amino acid and constitutes the C-terminal amino acid of the second RGD loop;
X₂₈ is selected from the group consisting of I, L and V, and is preferably I;
X₂₉ is selected from the group consisting of D, E, K, N, Q, and V, is preferably Q or K, and is more preferably Q;
X₃₀ is selected from the group consisting of C, G and P, and is preferably P;
X₃₁ is selected from the group consisting of I, L and V, is preferably L or V and is more preferably L;
X₃₂ is selected from the group consisting of D, E, S and T, is preferably E or T and is more preferably E;
X₃₃ is selected from the group consisting of D, E, S and T, is preferably E, or T, and is more preferably K; and
X₃₄ is selected from the group consisting of D and E, and is preferably D;

8. The polypeptide of claim 5, wherein the N-terminus of the V loop of fragment A is defined by the following sequence (from N-terminal to C-terminal):
(SEQ ID NO: 25)
X₃₅-X₃₆-X₃₇-X₃₈-X₃₉-X₄₀-X₄₁-X₄₂
wherein
X₃₅ is selected from the group consisting of F, Y, and W, and is preferably F;
X₃₈ is selected from the group consisting of H, K and R, and is preferably K;
X₃₇ is selected from the group consisting of A, V, I, and L, and is preferably A;
X₃₈ is selected from the group consisting of H, K, and R, and is preferably R;
X₃₉ is selected from the group consisting of A, V, I, and L, and is preferably V;
X₄₀ is selected from the group consisting of A, V, I, L and M, and is preferably M;
X₄₁ is selected from the group consisting of A, V, I, and L, and is preferably V; and
X₄₂ is any amino acid and constitutes the N-terminal amino acid of the V loop.

9. The polypeptide of claim 5, wherein the C-terminus of the V loop of fragment A is defined by the following sequence (from N-terminal to C-terminal):
(SEQ ID NO: 26)
X₄₃-X₄₄-X₄₅-X₄₆-X₄₇-X₄₈-X₄₉
wherein
X₄₃ is any amino acid and constitutes the C-terminal amino acid of the V loop;
X₄₄ is selected from the group consisting of F, Y, and W, and is preferably Y;
X₄₅ is selected from the group consisting of D, E, S and T, is preferably E or T and is more preferably E;
X₄₆ is selected from the group consisting of F, Y, and W, and is preferably W;
X₄₇ is selected from the group consisting of A, F, V, Y, and W, is preferably F or V and is more preferably F;
X₄₈ is selected from the group consisting of D, E, S and T, is preferably D or E and is more preferably E; and
X₄₉ is selected from the group consisting of F, Y, and W, and is preferably F.

10. The polypeptide of claim 2, wherein the heterologous modification is selected from the group consisting of one or more single amino acid mutations in comparison to the wildtype sequence of fragment A and/or B, one or more replacements of wildtype amino acid stretches by one or more heterologous amino acids and/or amino acid stretches one or more insertions of heterologous amino acid stretches, one or more deletions of one or more amino acids and one or more amino acid modifications as well as any combination(s) thereof.

11. The polypeptide of claim 2, wherein the heterologous modification provides a target specific binding entity.

12. The polypeptide of claim 11, wherein the target specific binding entity is selected from the group consisting of antigens, epitopes, CDRs, antibodies, antibody fragments such as an antigen binding (Fab) fragment, a Fab′ fragment, a F(ab′)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, a paratope, an alternative scaffold protein, and a fusion protein thereof, a toxin and a venom.

13. The polypeptide of claim 1 having one of the following amino acid sequences (from N-terminal to C-terminal):
(SEQ ID NO: 27)
MSYYHHHHHHDYDIPTTENLYFQGAMGSGIQPNVN
EYMFSNKFKARVMVSRKAPEGVTVNDTYDHKEDIL
KYEWFEFILPEGNFSATMTIDLMNNAIIDNYLEIG
RQNGVLESDIGVKFDTRNFRLGWDPETKLIMPGVY
TYEAFHPDIVLLPGCGVDFTESRLSNLLGIRKRHP
FQEGFKIMYEDLEGGNIPALLDVTAYEESKKDTTT
ETTTKKELKIQPLEKDSKSRSYNVLEDKINTAYRS
WYLSYNYGNPEKGIRSWTLLTTSDVTCGANGDSGN
PVFSKSFYNEQAVYSQQLRQATSLTHVFNRFPENQ
IURPPAPTITTVSENVP
(SEQ ID NO: 32)
GAMGSGIQPNVNEYMFSNKFKARVMVSRKAPEGVT
VNDTYDHKEDILKYEWFEFILPEGNFSATMTIDLM
NNAIIDNYLEIGRQNGVLESDIGVKFDTRNFRLGW
DPETKLIMPGVYTYEAFHPDIVLLPGCGVDFTESR
LSNLLGIRKRHPFQEGFKIMYEDLEGGNIPALLDV
TAYEESKKDTTTETTTKKELKIQPLEKDSKSRSYN
VLEDKINTAYRSWYLSYNYGNPEKGIRSWTLLTTS
DVTCGANGDSGNPVFSKSFYNEQAVYSQQLRQATS
LTHVFNRFPENQILIRPPAPTITTVSENVP
(SEQ ID NO: 27)
MSYYHHHHHHDYDIPTTENLYFQGTIMHTNMPNVN
EFMYSNKFKARVMVSRKAPEGVTVNDTYDHKEDIL
EYEWVEFELPEGNFSVTMTIDLMNNAIIDNYLAVG
RQNGVLESDIGVKFDTRNFRLGWDPVTELVMPGVY
TNEAFHPDIVLLPGCGVDFTEMSYYHHHHHHDYDI
PTTENLYFQGAMGSGIQPNVNEYMFSNKFKARVMV
SRKAPEGVTVNDTYDHKEDILKYEWFEFILPEGNF
SATMTIDLMNNAIIDNYLEIGRQNGVLESDIGVKF
DTRNFRLGWDPETKLIMPGVYTYEAFHPDIVLLPG
CGVDFTESRLSNLLGIRKRHPFQEGFKIMYEDLEG
GNIPALLDVTAYEESKKDTTTETTTKKELKIQPLE
KDSKSRSYNVLEDKINTAYRSWYLSYNYGNPEKGI
RSWTLLTTSDVTCGANGDSGNPVFSKSFYNEQAVY
SQQLRQATSLTHVFNRFPENQIURPPAPTITTVSE
NVP
(SEQ ID NO: 28)
GAMGSGIQPNVNEYMFSNKFKARVMVSRKAPEGVT
VNDTYDHKEDILKYEWFEFILPEGNFSATMTIDLM
NNAIIDNYLEIGRQNGVLESDIGVKFDTRNFRLGW
DPETKLIMPGVYTYEAFHPDIVLLPGCGVDFTESR
LSNLLGIRKRHPFQEGFKIMYEDLEGGNIPALLDV
TAYEESKKDTTTETTTKKELKIQPLEKDSKSRSYN
VLEDKINTSRLSNLLGIRKRQPFQEGFQIMYEDLE
GGNIPALLDVDAYEKSKKDTTTETTTKKELKIQPV
EKDSKDRSYNVLPDKINTAYRSWYLAYNYGDPEKG
VRSWTLLTTSDVTCGVEQAELLPVYSKSFFNEQAV
YSQQLRAFTSLTHVFNRFPENQILVRPPAPTITTV
SENVP
(SEQ ID NO: 33)
QGTIMHTNMPNVNEFMYSNKFKARVMVSRKAPEGV
TVNDTYDHKEDILEYEWVEFELPEGNFSVTMTIDL
MNNAIIDNYLAVGRQNGVLESDIGVKFDTRNFRLG
WDPVTELVMPGVYTNEAFHPDIVLLPGCGVDFTES
RLSNLLGIRKRQPFQEGFQIMYEDLEGGNIPALLD
VDAYEKSKKDTTTETTTKKELKIQPVEKDSKDRSY
NVLPDKINTAYRSWYLAYNYGDPEKGVRSWTLLTT
SDVTCGVEQAELLPVYSKSFFNEQAVYSQQLRAFT
SLTHVFNRFPENQILVRPPAPTITTVSENVP

14. An isolated engineered polypeptide comprising the large fragment of the alpha-helical domain of an adenovirus penton base protein which polypeptide lacks the small fragment of the alpha-helical domain and the jellyroll fold domain of the adenovirus penton base protein, wherein said large fragment optionally contains one or more heterologous modifications.

15. The polypeptide of claim 14 having, from N- to C-terminal, the structure of the following general formula II:
N-A-C (II)
wherein
A represents an amino acid stretch corresponding to the N-terminal amino acid stretch of the adenovirus penton base present between the first and the second amino acid stretch forming the jellyroll fold domain of the adenovirus penton base;
N may or may not be present, and, if present, represents a chemical group consisting of an amino acid, an oligopeptide and a polypeptide;
C: may or may not be present, and, if present, represents a chemical group consisting of an amino acid, an oligopeptide and a polypeptide;
wherein, optionally, fragment A contain one or more heterologous modifications.

16. The polypeptide of claim 15 wherein the fragment A comprises an amino acid sequence selected from the group consisting of the amino acid sequences according to the following table and amino acid sequences having an identity of at least 85% with the respective amino acid sequence shown in the following table:

| Sequence | Sequence | | N-terminal | C-terminal |
|---|---|---|---|---|
| based on | according to | | amino acid | amino acid |
| penton base | UniProt | SEQ ID | selected from | selected from |
| protomer of | Acc. No. | NO: | positions | positions |
| hAd3 | Q2Y0H9 | 1 | 130 to 137 | 399 to 405 |
| hAd2 | P03276 | 2 | 130 to 137 | 426 to 432 |
| hAd4 | Q2KSF3 | 3 | 126 to 133 | 380 to 386 |
| hAd5 | P12538 | 4 | 130 to 137 | 426 to 432 |
| hAd7 | Q9JFT6 | 5 | 130 to 137 | 399 to 405 |
| hAd11 | D2DM93 | 6 | 130 to 137 | 416 to 422 |
| hAd12 | P36716 | 7 | 120 to 127 | 352 to 358 |
| hAd17 | F1DT65 | 8 | 117 to 124 | 371 to 377 |
| hAd25 | M0QUK0 | 9 | 125 to 133 | 389 to 395 |
| hAd35 | Q7T941 | 10 | 131 to 138 | 446 to 452 |
| hAd37 | Q912J1 | 11 | 117 to 124 | 373 to 379 |
| hAd41 | F8WQN4 | 12 | 128 to 135 | 362 to 368 |
| gorAd | E5L3Q9 | 13 | 131 to 138 | 417 to 423 |
| ChimpAd | G9G849 | 14 | 126 to 133 | 373 to 379 |
| sAd18 | H8PFZ9 | 15 | 128 to 135 | 354 to 360 |
| sAd20 | F6KSU4 | 16 | 127 to 134 | 359 to 365 |
| sAd49 | F2WTK5 | 17 | 128 to 135 | 357 to 363 |
| rhAd51 | A0A0A1EWW1 | 18 | 125 to 132 | 353 to 359 |
| rhAd52 | A0A0A1EWX7 | 19 | 125 to 132 | 351 to 357 |
| rhAd53 | A0A0A1EWZ7 | 20 | 126 to 133 | 352 to 358 |

wherein, optionally, fragment A contains one or more heterologous modifications.

17. The polypeptide of claim 15 wherein fragment A contains one or more heterologous modifications wherein said one or more heterologous modifications is/are contained in the following sites:
the RGD loop region of fragment A; and/or
the V-loop of fragment A; and/or
the floor region having the sequence (from N- to C-terminal)
(SEQ ID NO: 21)
X₁-X₂-X₃-X₄-X₅-X₆-D-X₇-X₈-X₉-S-Y-
N-X₁₀-X₁₁-X₁₂-X₁₃-X₁₄-X₁₅-X₁₆
of fragment A, wherein
X₁ is I or L, and is preferably I;
X₂ is selected from the group consisting of K, Q and E, and is preferably Q;
X₃ is P or A, and is preferably P;
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably S;
X₈ is selected from the group consisting of K, T and S, and is preferably K;
X₉ is selected from the group consisting of K, S, N, G and D, and is preferably S;
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably D;
X₁₅ is selected from the group consisting of S, K, Q and T, and is preferably K; and
X₁₅ is selected from the group consisting of T, N, I, K and M, and is preferably I; and/or
the sequence (from N- to C-terminal) T-H-V-F-X₁₇-R-F-P (SEQ ID NO: 22) of fragment B wherein X₁₇ is D or N, and is preferably N.

18. The polypeptide of claim 17 wherein the N-terminus of the RGD loop region of fragment A is defined by the following sequence (from N-terminal to C-terminal):
(SEQ ID NO: 23)
X₁₈-X₁₉-X₂₀-X₂₁-X₂₂-X₂₃-X₂₄-X₂₅-X₂₆
wherein
X₁₈ is selected from the group consisting of D, E and N, and is preferably D;
X₁₉ is selected from the group consisting of V, L, and I, and is preferably V;
X₂₀ is any amino acid, preferably selected from the group consisting of A, D, E, K, S, and T, and is more preferably T;
X₂₁ is any amino acid, preferably selected from the group consisting of A, D, E, and K, and is more preferably A;
X₂₂ is selected from the group consisting of F, Y, and W, and is preferably Y;
X₂₃ is selected from the group consisting of A, D, E, N, and Q, is preferably E or Q, and is more preferably E;
X₂₄ is any amino acid, preferably selected from the group consisting of A, D, E, N, and K, and is more preferably E;
X₂₅ is selected from the group consisting of S or T, and is preferably S; and
X₂₆ is any amino acid and constitutes the N-terminal amino acid of the RGD loop region

19. The polypeptide of claim 17 wherein the C-terminus of the RGD loop region of fragment A is defined by the following sequence (from N-terminal to C-terminal):
(SEQ ID NO: 24)
X₂₇-X₂₈-X₂₉-X₃₀-X₃₁-X₃₂-X₃₃-X₃₄
Wherein
X₂₇ is any amino acid and constitutes the C-terminal amino acid of the second RGD loop;
X₂₈ is selected from the group consisting of I, L and V, and is preferably I;
X₂₉ is selected from the group consisting of D, E, K, N, Q, and V, is preferably Q or K, and is more preferably Q;
X₃₀ is selected from the group consisting of C, G and P, and is preferably P;
X₃₁ is selected from the group consisting of I, L and V, is preferably L or V and is more preferably L;
X₃₂ is selected from the group consisting of D, E, S and T, is preferably E or T and is more preferably E;
X₃₃ is selected from the group consisting of D, E, S and T, is preferably E, or T, and is more preferably K; and
X₃₄ is selected from the group consisting of D and E, and is preferably D;

20. The polypeptide of claim 17 wherein the N-terminus of the V loop of fragment A is defined by the following sequence (from N-terminal to C-terminal):
(SEQ ID NO: 25)
X₃₅-X₃₆-X₃₇-X₃₈-X₃₉-X₄₀-X₄₁-X₄₂
wherein
X₃₅ is selected from the group consisting of F, Y, and W, and is preferably F;
X₃₈ is selected from the group consisting of H, K and R, and is preferably K;
X₃₇ is selected from the group consisting of A, V, I, and L, and is preferably A;
X₃₈ is selected from the group consisting of H, K, and R, and is preferably R;
X₃₉ is selected from the group consisting of A, V, I, and L, and is preferably V;
X₄₀ is selected from the group consisting of A, V, I, L and M, and is preferably M;
X₄₁ is selected from the group consisting of A, V, I, and L, and is preferably V; and
X₄₂ is any amino acid and constitutes the N-terminal amino acid of the V loop.

21. The polypeptide of claim 17 wherein the C-terminus of the V loop of fragment A is defined by the following sequence (from N-terminal to C-terminal):
(SEQ ID NO: 26)
X₄₃-X₄₄-X₄₅-X₄₆-X₄₇-X₄₈-X₄₉
wherein
X₄₃ is any amino acid and constitutes the C-terminal amino acid of the V loop;
X₄₄ is selected from the group consisting of F, Y, and W, and is preferably Y;
X₄₅ is selected from the group consisting of D, E, S and T, is preferably E or T and is more preferably E;
X₄₆ is selected from the group consisting of F, Y, and W, and is preferably W;
X₄₇ is selected from the group consisting of A, F, V, Y, and W, is preferably F or V and is more preferably F;
X₄₈ is selected from the group consisting of D, E, S and T, is preferably D or E and is more preferably E; and
X₄₉ is selected from the group consisting of F, Y, and W, and is preferably F.

22. The polypeptide of claim 15 wherein the heterologous modification is selected from the group consisting of one or more single amino acid mutations in comparison to the wildtype sequence of fragment A, one or more replacements of wildtype amino acid stretches by one or more heterologous amino acids and/or amino acid stretches one or more insertions of heterologous amino acid stretches, one or more deletions of one or more amino acids and one or more amino acid modifications as well as any combination(s) thereof.

23. The polypeptide of claim 15, wherein the heterologous modification provides a target specific binding entity.

24. The polypeptide of claim 23, wherein the target specific binding entity is selected from the group consisting of antigens, epitopes, CDRs, antibodies, antibody fragments such as an antigen binding (Fab) fragment, a Fab′ fragment, a F(ab′)2 fragment, a heavy chain antibody, a single-domain antibody (sdAb), a single-chain fragment variable (scFv), a fragment variable (Fv), a VH domain, a VL domain, a single domain antibody, a nanobody, an IgNAR (immunoglobulin new antigen receptor), a di-scFv, a bispecific T-cell engager (BITEs), a dual affinity re-targeting (DART) molecule, a triple body, a diabody, a single-chain diabody, a paratope, an alternative scaffold protein, and a fusion protein thereof, a toxin and a venom.

25. A nucleic acid encoding the polypeptide of claim 1.

26. A vector comprising the nucleic acid of claim 25.

27. The vector of claim 26 containing the nucleic acid of claim 25 within an expression cassette.

28. A recombinant host cell comprising the vector of claim 26.

29. A method for the production of a polypeptide comprising the step of culturing the host cell of claim 28 under conditions allowing the expression of said polypeptide.

30. The method of claim 29 further comprising the step of purifying the polypeptide from the cultured host cells.

31. An engineered adenovirus penton base protein comprising the polypeptide of claim 5 fused to the multimerization domain (jellyroll fold domain) of an adenovirus penton base protein.

32. The penton base protein of claim 31 having, from N- to C-terminal, the structure of the following general formula III:
D-A-E-B-F (III)
wherein A and B are the fragments of the alpha-helical crown domain as defined in any one of claims 2 to 4, and D, E and F are the amino acid sequences of an adenovirus penton base forming the multimerization (jellyroll fold) domain, wherein one or more heterologous modifications is/are present in the floor region of fragment A and/or in the B loop of fragment B.

33. An engineered adenovirus penton base protein comprising the polypeptide of claim 15 fused to the multimerization domain (jellyroll fold domain) of an adenovirus penton base protein.

34. The penton base of claim 33 having, from N- to C-terminal, the structure of the following general formula IV:
D-A-E-Li-F (IV)
wherein A is the large fragment of the alpha-helical crown domain as defined in any one of claims 15 or 16, and D, E and F are the amino acid sequences of an adenovirus penton base forming the multimerization (jellyroll fold) domain, wherein, optionally and preferably, one or more heterologous modifications is/are present in the floor region of fragment A, and wherein Li is a linker selected from peptides, oligopeptides, polypeptides, proteins and protein complexes.

35. The penton base of claim 32 wherein fragment D of general formula (III) or (IV) has the following consensus sequence (SEQ ID NO: 34):
(U)₁₋₄₇ PTJ₁GRNSIRY SJ₂J₃x₄PJ₅J₆DTT J₇J₈YLVDNKSA DIASLNYQND HSNFJ₅TTVJ₉Q NNDJ₁₀J₁₁PJ₁₂EAJ₁₃ TQTINJ₁₄DJ₁₅RS RWGJ₁₆J₁₇LKTIJ₁₈ J₁₉TZ₁Z₂Z₃Z₄Z₅Z₆Z₇Z₈ Z₉Z₁₀Z₁₁Z₁₂Z₁₃Z₁₄Z₁₅
wherein: fragment D ends on the C-terminal side before Z₁ at residue T or at an amino acid from Z₁ to Z₁₅
U is any or no amino acid
J₁ is E or G
J₂ is E or S
J₃ is L or V
J₄ is A or S
J₅ is L or Q
J₆ is Y or E
J₇ is R or K
J₈ is V or L
J₉ is V or I
J₁₀ is F or Y
J₁₁ is T or S
J₁₂ is A or T or I or G
J₁₃ is S or G
J₁₄ is F or L
J₁₅ is E or D
J₁₆ is A or G
J₁₇ is D or Q
J₁₈ is L or M
J₁₉ is H or R
Z₁, if present, is N
Z₂, if present, is M
Z₃, if present, is P
Z₄, if present, is N
Z₅, if present, is V or I
Z₆, if present, is N
Z₇, if present, is E or D
Z₈, if present, is Y or F
Z₉, if present, is M
Z₁₀, if present, is F or S or Y
Z₁₁, if present, is T or S
Z₁₂, if present, is S or N
Z₁₃, if present, is K
Z₁₄, if present, is F
Z₁₆, if present, is K.

36. The penton base of claim 35 wherein the fragment D comprises an amino acid sequence selected from the group consisting of the amino acid sequences of the following table and amino acid sequences having and identity of at least 85% with the respective amino acid sequence shown in the following table:

| | Sequence | | N-terminal | C-terminal |
|---|---|---|---|---|
| Sequence based | according to | | amino acid | amino acid |
| on penton base | UniProt Acc. | SEQ | selected from | selected from |
| protomer of | No. | ID NO: | positions | positions |
| hAd3 | Q2Y0H9 | 1 | 1 to 48 | 129 to 144 |
| hAd2 | P03276 | 2 | 1 to 48 | 129 to 144 |
| hAd4 | Q2KSF3 | 3 | 1 to 44 | 125 to 140 |
| hAd5 | P12538 | 4 | 1 to 48 | 129 to 144 |
| hAd7 | Q9JFT6 | 5 | 1 to 48 | 129 to 144 |
| hAd11 | D2DM93 | 6 | 1 to 48 | 129 to 144 |
| hAd12 | P36716 | 7 | 1 to 38 | 119 to 134 |
| hAd17 | F1DT65 | 8 | 1 to 35 | 116 to 131 |
| hAd25 | M0QUK0 | 9 | 1 to 43 | 124 to 139 |
| hAd35 | Q7T941 | 10 | 1 to 49 | 130 to 145 |
| hAd37 | Q912J1 | 11 | 1 to 35 | 116 to 131 |
| hAd41 | F8WQN4 | 12 | 1 to 46 | 127 to 142 |
| gorAd | E5L3Q9 | 13 | 1 to 49 | 130 to 145 |
| ChimpAd | G9G849 | 14 | 1 to 44 | 125 to 140 |
| sAd18 | H8PFZ9 | 15 | 1 to 46 | 127 to 142 |
| sAd20 | F6KSU4 | 16 | 1 to 45 | 126 to 141 |
| sAd49 | F2WTK5 | 17 | 1 to 48 | 127 to 142 |
| rhAd51 | A0A0A1EWW1 | 18 | 1 to 43 | 124 to 139 |
| rhAd52 | A0A0A1EWX7 | 19 | 1 to 43 | 124 to 139 |
| rhAd53 | A0A0A1EWZ7 | 20 | 1 to 44 | 125 to 140 |

37. The penton base claim 32 wherein fragment E of general formula (III) or (IV) has the following sequence (SEQ ID NO: 35):
Z₁₇Z₁₈Z₁₉Z₂₀Z₂₁Z₂₂Z₂₃Z₂₄Z₂₅Z₂₆ Z₂₇QVYWSLPDJ₂₀ MJ₂₁DPVTFRST J₂₂QJ₂₃J₂₄NJ₂₅PVVGJ₂₆ ELZ₂₈Z₂₉Z₃₀
wherein: fragment E begins on the N-terminal side at an amino acid from Z₁₇ to Z₂₇ or at amino acid Q after Z₂₇;
amino acid stretch B ends on the C-terminal side before Z₂₈ at amino acid L or at an amino acid from Z₂₈ to Z₃₀;
Z₁₇, if present, is L or S
Z₁₈, if present, is T or P or C
Z₁₉, if present, is T or P
Z₂₀, if present, is P or S or A or R
Z₂₁, if present, is N or D
Z₂₂, if present, is G or V
Z₂₃, if present, is H or T
Z₂₄, if present, is C
Z₂₅, if present, is G
Z₂₆, if present, is A or V or S
Z₂₇, if present, is E or Q
J₂₀ is L or M
J₂₁ is Q or K
J₂₂ is Q or R or S
J₂₃ is V or I
J₂₄ is S or N
J₂₅ is Y or F
J₂₆ is A or V
Z₂₈, if present, is M or L
Z₂₉, if present, is P
Z₃₀, if present, is V or F.

38. The penton base of claim 37 wherein the fragment E comprises an amino acid sequence selected from the group consisting of the amino acid sequences of the following table and amino acid sequences having and identity of at least 85%, more preferred at least 90%, even more preferred 95%, particularly preferred at least 98%, most preferred at least 99%, with the respective amino acid sequence shown in the following table:

| | Sequence | | N-terminal | C-terminal |
|---|---|---|---|---|
| Sequence based | according to | | amino acid | amino acid |
| on penton base | UniProt | SEQ ID | selected from | selected from |
| protomer of | Acc. No. | NO: | positions | positions |
| hAd3 | Q2Y0H9 | 1 | 398 to 409 | 440 to 443 |
| hAd2 | P03276 | 2 | 425 to 436 | 467 to 470 |
| hAd4 | Q2KSF3 | 3 | 379 to 390 | 421 to 444 |
| hAd5 | P12538 | 4 | 425 to 436 | 467 to 470 |
| hAd7 | Q9JFT6 | 5 | 398 to 409 | 440 to 443 |
| hAd11 | D2DM93 | 6 | 415 to 426 | 457 to 460 |
| hAd12 | P36716 | 7 | 351 to 362 | 393 to 397 |
| hAd17 | F1DT65 | 8 | 370 to 381 | 413 to 416 |
| hAd25 | M0QUK0 | 9 | 388 to 399 | 440 to 443 |
| hAd35 | Q7T941 | 10 | 445 to 456 | 497 to 500 |
| hAd37 | Q912J1 | 11 | 372 to 383 | 414 to 417 |
| hAd41 | F8WQN4 | 12 | 362 to 373 | 404 to 407 |
| gorAd | E5L3Q9 | 13 | 416 to 427 | 458 to 461 |
| ChimpAd | G9G849 | 14 | 372 to 383 | 420 to 423 |
| sAd18 | H8PFZ9 | 15 | 353 to 364 | 395 to 398 |
| sAd20 | F6KSU4 | 16 | 358 to 369 | 400 to 403 |
| sAd49 | F2WTK5 | 17 | 356 to 367 | 398 to 401 |
| rhAd51 | A0A0A1EWW1 | 18 | 352 to 363 | 394 to 397 |
| rhAd52 | A0A0A1EWX7 | 19 | 350 to 361 | 392 to 395 |
| rhAd53 | A0A0A1EWZ7 | 20 | 351 to 362 | 393 to 396 |

39. The penton base of claim 32 wherein fragment F of general formula (III) or (IV) has the following sequence (SEQ ID NO: 36):
Z₃₁Z₃₂Z₃₃ALTDHGT LPLRSSIJ₂₇GV QRVTJ₂₈TDARR RTCPYVYKA LGIVJ₃₀PJ₃₁VLS SRTF
wherein: fragment F begins on the N-terminal side at an amino acid from Z₃₁ to Z₃₃ or at amino acid A after Z₃₃;
Z₃₁, if present, is N
Z₃₂, if present, is V
Z₃₃, if present, is P
J₂₇ is R or S or G
J₂₈ is V or I
J₂₉ is Y or H
J₃₀ is A or S
J₃₁ is R or K

40. The penton base of claim 39 wherein fragment F comprises an amino acid sequence selected from the group consisting of the amino acid sequences of the following table and amino acid sequences having and identity of at least 85% with the respective amino acid sequence shown in the following table:

| Sequence | Sequence | | N-terminal | C- |
|---|---|---|---|---|
| based on | according to | | amino | terminal |
| penton base | UniProt | SEQ | acid selected | amino acid |
| protomer of | Acc. No. | ID NO: | from positions | position |
| hAd3 | Q2Y0H9 | 1 | 492 to 495 | 544 |
| hAd2 | P03276 | 2 | 519 to 522 | 571 |
| hAd4 | Q2KSF3 | 3 | 466 to 469 | 535 |
| hAd5 | P12538 | 4 | 492 to 495 | 571 |
| hAd7 | Q9JFT6 | 5 | 465 to 468 | 544 |
| hAd11 | D2DM93 | 6 | 482 to 485 | 561 |
| hAd12 | P36716 | 7 | 419 to 422 | 497 |
| hAd17 | F1DT65 | 8 | 438 to 441 | 517 |
| hAd25 | M0QUK0 | 9 | 455 to 458 | 534 |
| hAd35 | Q7T941 | 10 | 522 to 525 | 561 |
| hAd37 | Q912J1 | 11 | 439 to 442 | 519 |
| hAd41 | F8WQN4 | 12 | 439 to 432 | 508 |
| gorAd | E5L3Q9 | 13 | 483 to 486 | 875 |
| ChimpAd | G9G849 | 14 | 445 to 458 | 532 |
| sAd18 | H8PFZ9 | 15 | 420 to 423 | 508 |
| sAd20 | F6KSU4 | 16 | 425 to 428 | 512 |
| sAd49 | F2WTK5 | 17 | 423 to 426 | 511 |
| rhAd51 | A0A0A1EWW1 | 18 | 419 to 422 | 505 |
| rhAd52 | A0A0A1EWX7 | 19 | 417 to 420 | 503 |
| rhAd53 | A0A0A1EWZ7 | 20 | 418 to 421 | 504 |

41. A pentameric complex of the engineered adenovirus penton base protein according to claim 32.

42. A virus-like particle (VLP) comprising 12 pentameric complexes of claim 41.

43. The polypeptide of claim 1 for use as a medicament.

44. A pharmaceutical composition comprising the engineered adenovirus penton base protein of claim 32, optionally together with at least one pharmaceutically acceptable carrier, excipient and/or diluent.

45. A method for producing the VLP of claim 42 comprising the step of incubating a solution of a penton base according to claim 32 under conditions allowing the assembly of the polypeptide into a VLP.

46. The polypeptide of claim 14 for use in the treatment and/or prevention of an infectious disease, an immune disease, tumour or cancer.

47. A method of identifying a binding sequence to a target molecule comprising the steps of:
(ia) preparing a library of vectors each containing a nucleotide sequence encoding a polypeptide having a candidate binding sequence in an expression cassette, each polypeptide encoded by said nucleotide sequence comprising a candidate binding sequence as a heterologous modification in one or more of RGD loop region and/or V loop and/or floor region and/or B loop and having the structure of the following general formula (I):
N-A-L-B-C (I)
wherein
A represents an amino acid stretch corresponding to the N-terminal amino acid stretch of the adenovirus penton base protein present between the first and the second amino acid stretch forming the jellyroll fold domain of the adenovirus penton base;
B represents an amino acid stretch corresponding to the C-terminal amino acid stretch of the adenovirus penton base protein inserted between the second and the third amino acid stretch forming the jellyroll fold domain of the adenovirus penton base protein;
L represents a chemical group selected from the group consisting of an amino acid, an oligopeptide and a polypeptide;
N may or may not be present, and, if present, represents a chemical group consisting of an amino acid, an oligopeptide and a polypeptide;
C: may or may not be present, and, if present, represents a chemical group consisting of an amino acid, an oligopeptide and a polypeptide;
wherein fragment A and/or B contain(s) one or more heterologous modifications
wherein said one or more heterologous modifications is/are contained in the following sites:
the RGD loop region of fragment A; and/or
the V-loop of fragment A; and/or
the floor region having the sequence (from N- to C-terminal)
(SEQ ID NO: 21)
X₁-X₂-X₃-X₄-X₅-X₆-D-X₇-X₈-X₉-S-Y-
N-X₁₀-X₁₁-X₁₂-X₁₃-X₁₄-X₁₅-X₁₆
of fragment A, wherein
X₁ is I or L, and is preferably I;
X₂ is selected from the group consisting of K, Q and E, and is preferably Q;
X₃ is P or A, and is preferably P;
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably S;
X₈ is selected from the group consisting of K, T and S, and is preferably K;
X₉ is selected from the group consisting of K, S, N, G and D, and is preferably S;
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably D;
X₁₅ is selected from the group consisting of S, K, Q and T, and is preferably K; and
X₁₆ is selected from the group consisting of T, N, I, K and M, and is preferably I; and/or
the sequence (from N- to C-terminal) T-H-V-F-X₁₇-R-F-P (SEQ ID NO: 22) of fragment B wherein X₁₇ is D or N, and is preferably N;
wherein the candidate binding sequence encoded by the nucleotide sequence in each vector is different such that the vectors contain a randomized library of nucleotide sequences encoding randomized candidate binding sequences; or
(ib) preparing a library of vectors each containing a nucleotide sequence encoding a polypeptide having a candidate binding sequence in an expression cassette, each polypeptide encoded by said nucleotide sequence comprising a candidate binding sequence as a heterologous modification in one or more of RGD loop region and/or V loop and/or floor region the polypeptide having from N- to C-terminal, the structure of the following general formula II:
N-A-C (II)
wherein
A represents an amino acid stretch corresponding to the N-terminal amino acid stretch of the adenovirus penton base present between the first and the second amino acid stretch forming the jellyroll fold domain of the adenovirus penton base;
N may or may not be present, and, if present, represents a chemical group consisting of an amino acid, an oligopeptide and a polypeptide;
C: may or may not be present, and, if present, represents a chemical group consisting of an amino acid, an oligopeptide and a polypeptide;
wherein the fragment A comprises an amino acid sequence selected from the group consisting of the amino acid sequenoes according to the following table and amino acid sequences having an identity of at least 85% with the respective amino acid sequence shown in the following table:

| | Sequence | | N-terminal | C-terminal |
|---|---|---|---|---|
| Sequence based | according to | | amino acid | amino acid |
| on penton base | UniProt | SEQ ID | selected from | selected from |
| protomer of | Acc. No. | NO: | positions | positions |
| hAd3 | Q2Y0H9 | 1 | 130 to 137 | 399 to 405 |
| hAd2 | P03276 | 2 | 130 to 137 | 426 to 432 |
| hAd4 | Q2KSF3 | 3 | 126 to 133 | 380 to 386 |
| hAd5 | P12538 | 4 | 130 to 137 | 426 to 432 |
| hAd7 | Q9JFT6 | 5 | 130 to 137 | 399 to 405 |
| hAd11 | D2DM93 | 6 | 130 to 137 | 416 to 422 |
| hAd12 | P36716 | 7 | 120 to 127 | 352 to 358 |
| hAd17 | F1DT65 | 8 | 117 to 124 | 371 to 377 |
| hAd25 | M0QUK0 | 9 | 125 to 133 | 389 to 395 |
| hAd35 | Q7T941 | 10 | 131 to 138 | 446 to 452 |
| hAd37 | Q912J1 | 11 | 117 to 124 | 373 to 379 |
| hAd41 | F8WQN4 | 12 | 125 to 135 | 362 to 368 |
| gorAd | E5L3Q9 | 13 | 131 to 138 | 417 to 423 |
| ChimpAd | G9G849 | 14 | 126 to 133 | 373 to 379 |
| sAd18 | H8PFZ9 | 15 | 128 to 135 | 354 to 360 |
| sAd20 | F6KSU4 | 16 | 127 to 134 | 359 to 365 |
| sAd49 | F2WTK5 | 17 | 128 to 135 | 357 to 363 |
| rhAd51 | A0A0A1EWW1 | 18 | 125 to 132 | 353 to 359 |
| rhAd52 | A0A0A1EWX7 | 19 | 125 to 132 | 351 to 357 |
| rhAd53 | A0A0A1EWZ7 | 20 | 126 to 133 | 352 to 358 |

wherein fragment A contains one or more heterologous modifications wherein said one or more heterologous modifications is/are contained in the following sites:
the RGD loop region of fragment A; and/or
the V-loop of fragment A; and/or
the floor region having the sequence (from N- to C-terminal)
(SEQ ID NO: 21)
X₁-X₂-X₃-X₄-X₅-X₆-D-X₇-X₈-X₉-S-Y-
N-X₁₀-X₁₁-X₁₂-X₁₃-X₁₄-X₁₅-X₁₆
of fragment A, wherein
X₁ is I or L, and is preferably I;
X₂ is selected from the group consisting of K, Q and E, and is preferably Q;
X₃ is P or A, and is preferably P;
X₄ is selected from the group consisting of L, V and I, and is preferably L
X₅ is selected from the group consisting of T, E, A, K and L, and is preferably E;
X₆ is selected from the group consisting of E, K, T and Q, and is preferably K;
X₇ is selected from the group consisting of S, P and D, and is preferably S;
X₈ is selected from the group consisting of K, T and S, and is preferably K;
X₉ is selected from the group consisting of K, S, N, G and D, and is preferably S;
X₁₀ is L or V, and is preferably V;
X₁₁ is I or L, and is preferably I;
X₁₂ is selected from the group consisting of S, E and P, and is preferably E;
X₁₃ is no amino acid (i.e. not present) or is N, and is preferably no amino acid;
X₁₄ is D or G, and is preferably D;
X₁₅ is selected from the group consisting of S, K, Q and T, and is preferably K; and
X₁₆ is selected from the group consisting of T, N, I, K and M, and is preferably I; and/or
the sequence (from N- to C-terminal) T-H-V-F-X₁₇-R-F-P (SEQ ID NO: 22) of fragment B wherein X₁₇ is D or N, and is preferably N;
wherein the candidate binding sequence encoded by the nucleotide sequence in each vector is different such that the vectors contain a randomized library of nucleotide sequences encoding randomized candidate binding sequences
(ii) expressing the polypeptides encoded by the nucleotide sequences from the library of vectors of step (ia) or ib) in a host cell or a cell-free system, preferably cell free system;
(iii) contacting the polypeptides expressed in step (ii), optionally after purification from the host cells or the cell-free system, preferably cell free system, with the target molecule; and
(iv) detecting which polypeptide(s) have/has bound to the target molecule.

48. The method of claim 47 further comprising the step of determining the dissociation constant(s) (Kd) of the polypeptide(s) bound to the target molecule.
